(19) United States
(12) Patent Application Publication
(10) Pub. No.: US 2024/0174671 A1
(43) Pub. Date: May 30, 2024

(54) New 2,3-Dihydro-1H-pyrrolo[3,2-b]pyridine Derivatives as Sigma Ligands

(71) Applicant: ACONDICIONAMIENTO TARRASENSE, TERRASSA(Barcelona) (ES)

(72) Inventors: Marina VIRGILI-BERNADÓ, BARCELONA (ES); Mónica ALONSO-XALMA, BARCELONA (ES); Carmen ALMANSA-ROSALES, BARCELONA (ES); Jose Luís DÍAZ-FERNÁNDEZ, MANRESA (BARCELONA) (ES); Adriana LORENTE-CRIVILLÉ, BARCELONA (ES)

(73) Assignee: ACONDICIONAMIENTO TARRASENSE, TERRASSA(Barcelona) (ES)

(21) Appl. No.: 18/549,907

(22) PCT Filed: Mar. 8, 2022
(86) PCT No.: PCT/EP2022/055814

§ 371 Date: Sep. 9, 2023

(30) Foreign Application Priority Data
Mar. 10, 2021 (EP) 21382195.2

(51) Int. Cl. C07D 471/04; A61K 31/437; C07D 519/00

(52) U.S. Cl. CPC C07D 471/04; A61K 31/437; C07D 519/00

(57) ABSTRACT

The present invention relates to new 2,3-dihydro-1H-pyrrolo[3,2-b]pyridine derivatives of formula (I):

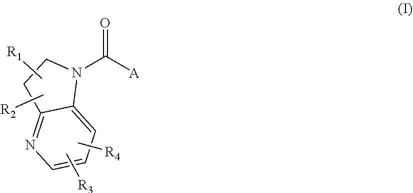

as sigma ligands having a great affinity for sigma receptors, especially sigma-1 (σ₁) and/or sigma-2 (σ₂) receptors, as well as to the process for the preparation thereof, to compositions comprising them, and to their use as medicaments.

### FIELD OF THE INVENTION

The present invention relates to new 2,3-dihydro-1H-pyrrolo[3,2-b]pyridine derivatives as sigma ligands having a great affinity for sigma receptors, especially sigma-1 (σ₁) and/or sigma-2 (σ₂) receptors, as well as to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (a) receptors, originally discovered in the central nervous system (CNS) of mammals in 1976 and initially related to the dysphoric, hallucinogenic and cardiac stimulant effects of opioids. Subsequent studies established a complete distinction between the a receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimcazole clinically shows effects in the treatment of psychosis [Snyder, S. H., Largent, B. L., J. Neuropsychiatry, (1989), 1,7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site, and has micromolar affinity for the sigma-2 (02) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimcazole, among others, known ligands with analgesic, anxiolytic, antidepressive, antiamnesic, antipsychotic and neuroprotective activity. Hence, the al receptor has possible physiological roles in processes related to analgesia, anxiety, addiction, amnesia, depression, schizophrenia, stress, neuroprotection and psychosis [Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Diaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the p-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of 02 receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some 02 ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that 02 ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of AP oligomers with the 02 receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vazquez-Rosa, E. et al., ACS Chem. Neurosci., (2019), 10, 1595-1602]. The 02 receptor has been implicated in other neurological disorders as schizophrenia [Harvey, P. D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ₁. Several σ₁-selective ligands are available, but ligands with high selectivity for σ₂ over σ₁ are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new 2,3-dihydro-1H-pyrrolo[3,2-b]pyridine derivatives with general Formula (I) show a selective affinity for sigma receptors, in particular, for σ₁ and/or σ₂ receptors. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders.

The invention is directed in a main aspect to a compound of Formula (I),

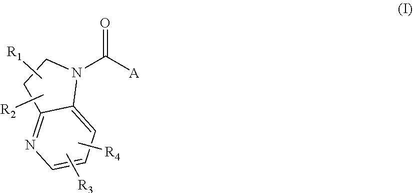

wherein R₁, R₂, R₃, R₄, and A are as defined below in the detailed description.

A further aspect of the invention refers to the processes for preparation of compounds of formula (I).

A still further aspect of the invention refers to the use of intermediate compounds for the preparation of a compound of formula (I).

It is also an aspect of the invention a pharmaceutical composition comprising a compound of formula (I).

Finally, it is an aspect of the invention a compound of formula (I) for use in therapy and more particularly for the treatment of pain and pain related conditions.

### DETAILED DESCRIPTION OF THE INVENTION

The invention is directed to a family of compounds, in particular, to 2,3-dihydro-1H-pyrrolo[3,2-b]pyridine derivatives which show a pharmacological activity towards the sigma receptors thus, solving the above problem of identifying alternative or improved pain treatments by offering such compounds.

The applicant has found that the problem of providing a new effective and alternative solution for treating pain and pain related disorders can surprisingly be solved by using an analgesic approach using compounds binding to the sigma receptors.

In a first aspect, the present invention is directed to a compound of formula (I):

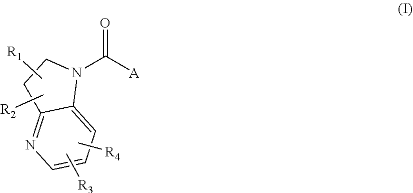

wherein
R₁ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₂ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₃ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl, CN and OR₃′;
   wherein R₃′ is unsubstituted or substituted C₁₋₆ alkyl or hydrogen;
R₄ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl and CN;
A is a linear or cyclic amine selected from one of the following groups:

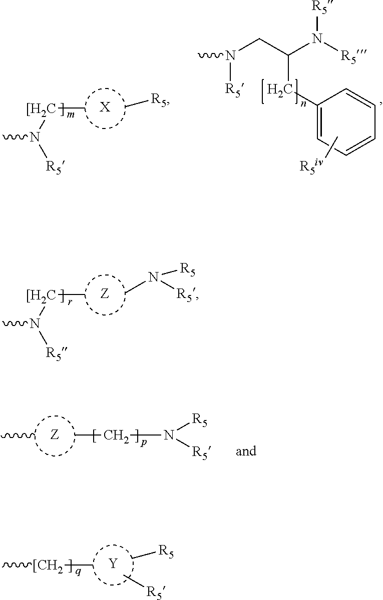

wherein
X is a N-containing heterocyclyl, wherein said heterocyclyl is a saturated heterocyclyl;
Y is a N-containing heterocyclyl, wherein said heterocyclyl is a saturated heterocyclyl containing 1 to 2 nitrogen atoms;
Z is a C₄₋₆-cycloalkyl or an N-containing heterocyclyl, wherein said heterocyclyl is a saturated heterocyclyl;
m is 0, 1 or 2;
n is 0, 1 or 2;
p is 0, 1 or 2;
q is 0, 1 or 2;
r is 0, 1 or 2;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
R₅′ is selected from the group consisting of hydrogen and a substituted or unsubstituted C₁₋₆ alkyl;
R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
   alternatively, R₅″ and R₅′″ may form, together with the nitrogen atom to which they are attached, a substituted or unsubstituted N-containing heterocyclyl; and
R₅^{iv} is selected from the group consisting of hydrogen, halogen and OR₆; wherein
   R₆ is substituted or unsubstituted C₁₋₆ alkyl or a hydrogen;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

The compounds of the invention represented by the above described formula (I) may include enantiomers depending on the presence of chiral centres or isomers depending on the presence of multiple bonds. The single isomers, enantiomers or diastereoisomers and mixtures thereof fall within the scope of the present invention.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt or solvate thereof.

For the sake of clarity the expression “a compound according to formula (I), wherein R₁, R₂, R₃, R₄, R₅, R₅′, R₅″, R₅′″, R₅^{iv}, R₆, X, Y, Z, m, n, p, q and r are as defined below in the detailed description” would (just like the expression “a compound of formula (I) as defined in the claims) refer to “a compound according to formula (I)”, wherein the definitions of the respective substituents R₁ etc. (also from the cited claims) are applied.

For clarity purposes, all groups and definitions described in the present description and referring to compounds of formula (I), also apply to all intermediates of synthesis.

In the context of this invention, alkyl is understood as meaning a straight or branched hydrocarbon chain radical containing no unsaturation, and which is attached to the rest of the molecule by a single bond. It may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl and C₁₋₆-alkyl represents C1-, C2-, C3-, C4-, C5- or C6-alkyl. Examples of alkyl radicals include among others methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl. If substituted by cycloalkyl, it corresponds to a “cycloalkylalkyl” radical, such as cyclopropylmethyl. If substituted by aryl, it corresponds to an “arylalkyl” radical, such as benzyl, benzhydryl or phenethyl. If substituted by heterocyclyl, it corresponds to a “heterocyclylalkyl” radical. Preferably alkyl is understood in the context of this invention C₁₋₆-alkyl like methyl, ethyl, propyl, butyl, pentyl, or hexyl; and more preferably is C₁₋₄-alkyl like methyl, ethyl, propyl or butyl.

Alkenyl is understood as meaning straight or branched hydrocarbon chain radical containing at least two carbon atoms and at least one unsaturation, and which is attached to the rest of the molecule by a single bond. It may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₆-alkenyl like ethylene, propylene, butylene, pentylene, or hexylene; or is C₂₋₄-alkenyl, like ethylene, propylene, or butylenes.

Alkynyl is understood as meaning a straight or branched hydrocarbon chain radical containing at least two carbon atoms and at least one carbon-carbon triple bond, and which is attached to the rest of the molecule by a single bond. It may be unsubstituted or substituted once or several times. It encompasses groups like e.g. —C═C—CH₃ (1-propynyl). Preferably alkynyl in the context of this invention is C₂₋₆-alkynyl like ethyne, propyne, butyene, pentyne, or hexyne; or is C₂₋₄-alkynyl like ethyne, propyne or butyene.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen, cycloalkyl, heterocyclyl, —OR′, —SR′, —SOR′, —SO₂R′, —CN, —COR′, —COOR′, —NR′R″, —CONR′R″, haloalkyl, haloalkoxy or —OC₁₋₆ alkyl wherein each of the R′ and R″ groups is independently selected from the group consisting of hydrogen, and C₁₋₆ alkyl.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CH₂—CH₂F, —CH₂—CHF₂, —CHCl₂, —CHF₂, and −CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OOCl₃, —OCF₃ and —OCH₂—CHCl₂. Preferably haloalkoxy is understood in the context of this invention as halogen-substituted —OC₁₋₄-alkyl representing halogen substituted C1-, C2-, C3- or C4-alkoxy. The halogen-substituted O-alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention, cycloalkyl is understood as meaning saturated and unsaturated (but not aromatic) cyclic hydrocarbons (without a heteroatom in the ring), which can be unsubstituted or once or several times substituted. Preferred cycloalkyls are C₃₋₄-cycloalkyl representing C3- or C4-cycloalkyl, C₃₋₅-cycloalkyl representing C3-, C4- or C5-cycloalkyl, C₃₋₆-cycloalkyl representing C3-, C4-, C5- or C6-cycloalkyl, C₃₋₇-cycloalkyl representing C3-, C4-, C5-, C6- or C7-cycloalkyl, C₃₋₈-cycloalkyl representing C3-, C4-, C5-, C6-, C7- or C8-cycloalkyl, C₄₋₅-cycloalkyl representing C4- or C5-cycloalkyl, C₄₋₆-cycloalkyl representing C4-, C5- or C6-cycloalkyl, C₄₋₇-cycloalkyl representing C4-, C5-, C6- or C7-cycloalkyl, C₅₋₆-cycloalkyl representing C5- or C6-cycloalkyl and C₅₋₇-cycloalkyl representing C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl. Preferably in the context of this invention cycloalkyl is C₃₋₈-cycloalkyl like cyclopropyl, cyclobutyl, cyclopentyl, cyclohexyl, cycloheptyl, or cyclooctyl; or is C₃₋₇-cycloalkyl like cyclopropyl, cyclobutyl, cyclopentyl, cyclohexyl, or cycloheptyl; or is C₃₋₆-cycloalkyl like cyclopropyl, cyclobutyl, cyclopentyl or cyclohexyl, especially cyclopentyl or cyclohexyl.

Aryl is understood as meaning 6 to 18 membered mono or fused polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphthyl or anthracenyl, more preferably the aryl is phenyl.

A ring system is a system consisting of at least one ring of connected atoms but including also systems in which two or more rings of connected atoms (polycyclic rings) are joined with “joined” meaning that the respective rings are sharing one (like a spiro structure), two or more atoms being a member or members of both joined rings.

A heterocyclyl radical or group (also called heterocyclyl hereinafter) is understood as meaning 4 to 18 membered mono or fused polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or fused polycyclic heterocyclic ring system of one or more rings of which at least one aromatic ring contains one or more heteroatoms from the group consisting of nitrogen, oxygen and/or sulfur in the ring; preferably it is a 5 to 18 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 4 to 18 membered mono or fused polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably it is a 4 to 18 membered mono or fused polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably it is selected from azetidine, oxetane, tetrahydrofuran, oxazepam, pyrrolidine, piperidine, piperazine, tetrahydropyran, morpholine, indoline, oxopyrrolidine, benzodioxane, especially is piperazine, benzodioxane, morpholine, tetrahydropyran, piperidine, oxopyrrolidine and pyrrolidine.

Preferably, in the context of this invention heterocyclyl is defined as a 4 to 18 membered mono or fused polycyclic ring system of one or more saturated or unsaturated rings of which at least one ring contains one or more heteroatoms selected from the group consisting of nitrogen, oxygen and/or sulfur in the ring. Preferably it is a 4 to 18 membered mono or fused polycyclic heterocyclic ring system of one or two saturated or unsaturated rings of which at least one ring contains one or more heteroatoms selected from the group consisting of nitrogen, oxygen, and sulfur in the ring. More preferably, it is a 4 to 12 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of heterocyclyls include azetidine, azepane, oxetane, tetrahydrofuran, oxazepam, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole and quinazoline, 3,9-diazaspiro[5.5]undecane, 2,8-diazaspiro[4.5]decane, 2,7-diazaspiro[3.5]nonane, 2,7-diazaspiro[4.4]nonane, octahydropyrrolo[3,4-c]pyrrole, especially is pyridine, piperazine, pyrazine, indazole, benzodioxane, thiazole, benzothiazole, morpholine, tetrahydropyran, pyrazole, imidazole, piperidine, thiophene, indole, benzimidazole, pyrrolo[2,3-b]pyridine, benzoxazole, oxopyrrolidine, pyrimidine, oxazepane, pyrrolidine, azetidine, azepane, oxetane, tetrahydrofuran, 3,9-diazaspiro[5.5]undecane, 2,8-diazaspiro[4.5]decane and 2,7-diazaspiro[3.5]nonane.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3-b]pyridine, quinoline, quinolone, isoquinoline, tetrahydrothienopyridine, phthalazine, benzo-1,2,5-thiadiazole, indole, benzotriazole, benzoxazole oxopyrrolidine, carbazole, thiazole, 3,9-diazaspiro[5.5]undecane, 2,8-diazaspiro[4.5]decane, 2,7-diazaspiro[3.5]nonane, 2,7-diazaspiro[4.4]nonane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle.

If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) once or several times being connected to another atom through 1 to 4 (—CH₂—) groups. Most preferably alkylaryl is benzyl (i.e. —CH₂-phenyl).

In the context of this invention alkylheterocyclyl is understood as meaning a heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine, —CH₂-tetrahydropyran and —CH₂CH₂-tetrahydropyran.

In the context of this invention alkylcycloalkyl is understood as meaning a cycloalkyl group being connected to another atom through a C₁₋₆-alkyl (see above) which may be branched or linear and is unsubstituted or substituted once or several times. Preferably alkylcycloalkyl is understood as meaning a cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 6 or 7 membered monocyclic aryl. Even more preferably the aryl is a 6 membered monocyclic aryl, preferably phenyl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl. In another preferred embodiment, said non-aromatic heterocyclyl is a bicyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

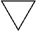

(leading to a spiro structure) and/or with ═O.

Moreover, in connection with cycloalkyl (including alkyl-cycloalkyl), or heterocyclyl (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl is spirosubstituted or substituted with ═O.

The term “leaving group” means a molecular fragment that departs with a pair of electrons in heterolytic bond cleavage. Leaving groups can be anions or neutral molecules. Common anionic leaving groups are halides such as Cl-, Br-, and I-, and sulfonate esters, such as tosylate (TsO—), mesylate, nosylate or triflate.

The term “salt” is to be understood as meaning any form of the active compound used according to the invention in which it assumes an ionic form or is charged and is coupled with a counter-ion (a cation or anion) or is in solution. By salt is also to be understood complexes of the active compound with other molecules and ions, in particular complexes via ionic interactions. The definition particularly includes physiologically acceptable salts, this term must be understood as equivalent to “pharmacologically acceptable salts”.

The term “physiologically acceptable salt” means in the context of this invention any salt that is physiologically tolerated (most of the time meaning not being toxic-especially lacking toxicity caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this it is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to formula (I) defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to formula (I) defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to formula (I) defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OS₂R′, OSO₃R′, NO₂, NHR′, NR′R″, ═N—R′, N(R′)COR′, N(COR′)₂, N(R)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R)SO₂R′, PO(OR′)₂, PO(OR′)R′, PO(OR′)(N(R′)R′), C₁₋₁₂ alkyl, C₃₋₁₀ cycloalkyl, C₂₋₂ alkenyl, C₂₋₂ alkynyl, aryl, and heterocyclic group, wherein each of the R′ and R″ groups is independently selected from the group consisting of hydrogen, C₁₋₁₂ alkyl, C₃₋₁₀ cycloalkyl, C₂₋₂ alkenyl, C₂₋₂ alkynyl, aryl and heterocyclic group. Where such groups are themselves substituted, the substituents may be chosen from the foregoing list.

In a particular embodiment of the invention, the compound of formula (I) according to the invention is a compound of formula (Ia):

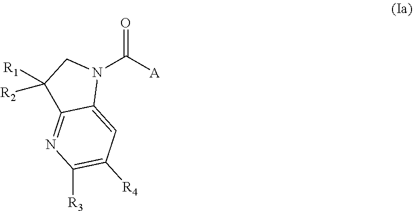

wherein R₁, R₂, R₃, R₄, and A are as defined before for a compound of formula (I);
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
R₁ is selected from the group consisting of hydrogen, unsubstituted or substituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the invention, the compound of formula (I) or (Ia) according to the invention is a compound wherein
R₁ is selected from the group consisting of hydrogen and unsubstituted or substituted C₁₋₆ alkyl, preferably ethyl or methyl; more preferably, methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
R₂ is selected from the group consisting of hydrogen, unsubstituted or substituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the invention, the compound of formula (I) or (Ia) according to the invention is a compound wherein
R₂ is selected from the group consisting of hydrogen and unsubstituted or substituted C₁₋₆ alkyl; preferably ethyl or methyl; more preferably, methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the invention, the compound of formula (I) or (Ia) according to the invention is a compound wherein
R₃ is selected from the group consisting of hydrogen, halogen, unsubstituted or substituted C₁₋₆ alkyl, unsubstituted or substituted cycloalkyl, unsubstituted or substituted heterocyclyl, CN and OR_{3′};
   wherein R₃′ is unsubstituted or substituted C₁₋₆ alkyl; preferably unsubstituted C₁₋₆ alkyl, more preferably, methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the invention, the compound of formula (I) or (Ia) according to the invention is a compound wherein
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
R₄ is selected from the group consisting of hydrogen, halogen, preferably fluorine or chlorine, unsubstituted or substituted C₁₋₆ alkyl, preferably methyl, and CN;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the invention, the compound of formula (I) or (Ia) according to the invention is a compound wherein
A is an amine according to the following group:

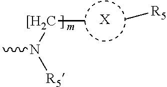

wherein
X is a N-containing heterocyclyl, wherein said heterocyclyl is a saturated heterocyclyl;
m is 0, 1, or 2; preferably m is 0 or 1;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl; and
R₅′ is selected from the group consisting of hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the invention, the compound of formula (I) or (Ia) according to the invention is a compound wherein
A is an amine according to the following group:

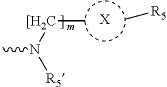

wherein
X is a N-containing heterocyclyl wherein said heterocyclyl is a mono or polycyclic saturated heterocyclyl containing only one nitrogen atom;
m is 0, 1, or 2; preferably m is 0 or 1;
R₅ is selected from the group consisting of substituted or unsubstituted C₁₋₆ alkyl, substituted or unsubstituted alkylaryl, substituted or unsubstituted heterocyclyl, and substituted or unsubstituted alkylheterocyclyl; and
R₅′ is hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the invention, the compound of formula (I) or (Ia) according to the invention is a compound wherein
A is an amine according to the following group:

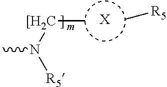

wherein
X is a N-containing heterocyclyl wherein said heterocyclyl is a mono or polycyclic saturated heterocyclyl containing only one nitrogen atom;
m is 0 or 1;
R₅ is selected from the group consisting of substituted or unsubstituted C₁₋₄ alkyl, substituted or unsubstituted alkylaryl, preferably, substituted or unsubstituted C₁₋₄ alkyl-phenyl, more preferably CH₂—CH₂-phenyl or CH₂-phenyl (benzyl), and substituted or unsubstituted alkylheterocyclyl; and
R₅′ is hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the invention, the compound of formula (I) or (Ia) according to the invention is a compound wherein
A is an amine according to the following group:

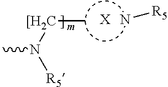

wherein
X is a N-containing heterocyclyl wherein said heterocyclyl is mono or polycyclic saturated heterocyclyl containing only one nitrogen atom, said nitrogen atom being directly linked to R₅;
m is 0 or 1;
R₅ is selected from the group consisting of substituted or unsubstituted C₁₋₄ alkyl, substituted or unsubstituted alkylaryl, preferably, substituted or unsubstituted C₁₋₄ alkyl-phenyl, more preferably CH₂—CH₂-phenyl or CH₂-phenyl (benzyl), and substituted or unsubstituted alkylheterocyclyl, preferably, alkyl-O-containing heterocyclyl; and
R₅′ is hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the invention, the compound of formula (I) or (Ia) according to the invention is a compound wherein
A is an amine according to the following group:

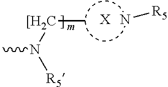

wherein
X is a N-containing heterocyclyl wherein said heterocyclyl is monocyclic saturated heterocyclyl containing only one nitrogen atom, said nitrogen atom being directly linked to R₅;
m is 0 or 1;
R₅ is selected from the group consisting of substituted or unsubstituted C₁₋₄ alkyl, substituted or unsubstituted alkylaryl, preferably, substituted or unsubstituted C₁₋₄ alkyl-phenyl, more preferably CH₂—CH₂-phenyl or CH₂-phenyl (benzyl), and substituted or unsubstituted alkylheterocyclyl, preferably, alkyl-O-containing heterocyclyl; and
R₅′ is hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular and preferred embodiment of the invention X is represented in the compound of formula (I) or (Ia) by one of the following moieties:

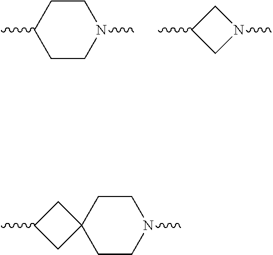

In another particular embodiment of the invention, the compound of formula (I) or (Ia) according to the invention is a compound wherein
A is a linear amine according to the following group:

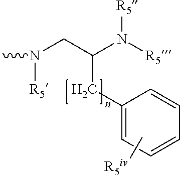

wherein:
n is 0 or 1;
R₅″ and R₅′″ are independently selected from the group consisting of hydrogen and substituted or unsubstituted C₁₋₆ alkyl; preferably unsubstituted C₁₋₆ alkyl, more preferably, unsubstituted C₁₋₃ alkyl;
   alternatively, R₅″ and R₅′″ may form, together with the nitrogen atom to which they are attached, a substituted or unsubstituted N-containing heterocyclyl;
R₅^{iv} is selected from the group consisting of hydrogen, halogen, preferably fluorine, and OR₆; wherein
   R₆ is substituted or unsubstituted alkyl, preferably unsubstituted C₁₋₆ alkyl, more preferably methyl; and
R₅′ is selected from the group consisting of hydrogen and a non-substituted C₁₋₆ alkyl, preferably methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is a linear amine according to the following group:

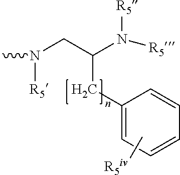

wherein:
n is 0 or 1;
R₅″ and R₅′″ are independently selected from the group consisting of hydrogen and unsubstituted C₁₋₃ alkyl;
   alternatively, R₅″ and R₅′″ may form, together with the nitrogen atom to which they are attached, a substituted or unsubstituted N-containing heterocyclyl;
R₅^{iv} is selected from the group consisting of hydrogen, halogen, preferably fluorine, and OR₆; wherein
   R₆ is unsubstituted C₁₋₆ alkyl, more preferably methyl; and
R₅′ is selected from the group consisting of hydrogen and a non-substituted C₁₋₆ alkyl, preferably methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

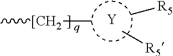

wherein
q is 0, 1 or 2; preferably, q is 0 or 1;
Y is a N-containing heterocyclyl, wherein said heterocyclyl is a saturated heterocyclyl containing 1 to 2 nitrogen atoms;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl; and
R₅′ is selected from the group consisting of hydrogen and a non-substituted C₁₋₆ alkyl, preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

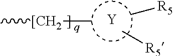

wherein
q is 0, 1 or 2; preferably, q is 0 or 1;
Y is a N-containing heterocyclyl, wherein said heterocyclyl is a mono or polycyclic saturated heterocyclyl containing 1 to 2 nitrogen atoms; wherein when said heterocyclyl is a polycyclic heterocyclyl then it can only contain one nitrogen atom per ring;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl; and
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

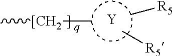

wherein
q is 0 or 1;
Y is a N-containing heterocyclyl, wherein said heterocyclyl is a mono or bicyclic saturated heterocyclyl containing 1 to 2 nitrogen atoms; wherein when said heterocyclyl is a bicyclic heterocyclyl then it contains one nitrogen atom per ring;
R₅ is selected from the group consisting of substituted or unsubstituted C₁₋₆ alkyl, substituted or unsubstituted alkylaryl, preferably CH₂-phenyl, substituted or unsubstituted alkylcycloalkyl, substituted or unsubstituted heterocyclyl, preferably unsubstituted O-containing heterocyclyl, and substituted or unsubstituted alkylheterocyclyl, preferably N-containing or O-containing heterocyclyl; and
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In more particular embodiment of the invention, the compound of formula (I) or (Ia) according to the invention is a compound wherein
A is an amine according to the following group:

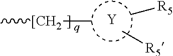

wherein
q is 0;
Y is a N-containing heterocyclyl, wherein said heterocyclyl is a mono or bicyclic saturated heterocyclyl containing 1 to 2 nitrogen atoms; wherein when said heterocyclyl is a bicyclic heterocyclyl then it contains one nitrogen atom per ring;
R₅ is selected from the group consisting of substituted or unsubstituted C₁₋₆ alkyl, substituted or unsubstituted alkylaryl, preferably CH₂-phenyl, substituted or unsubstituted alkylcycloalkyl, substituted or unsubstituted heterocyclyl, preferably unsubstituted O-containing heterocyclyl, and substituted or unsubstituted alkylheterocyclyl, preferably N-containing or O-containing heterocyclyl; and
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In more particular embodiment of the invention, the compound of formula (I) or (Ia) according to the invention is a compound wherein
A is an amine according to the following group:

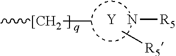

wherein
q is 0;
Y is a N-containing heterocyclyl, wherein said heterocyclyl is a mono or bicyclic saturated heterocyclyl containing 1 to 2 nitrogen atoms; wherein when said heterocyclyl is a bicyclic heterocyclyl then it contains one nitrogen atom per ring; and R₅ is directly attached to one of said nitrogen atoms.
R₅ is selected from the group consisting of substituted or unsubstituted C₁₋₆ alkyl, substituted or unsubstituted alkylaryl, preferably CH₂-phenyl, substituted or unsubstituted alkylcycloalkyl, substituted or unsubstituted heterocyclyl, preferably unsubstituted O-containing heterocyclyl, and substituted or unsubstituted alkylheterocyclyl, preferably N-containing or O-containing heterocyclyl; and
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular and preferred embodiment of the invention Y is represented in the compound of formula (I) or (Ia) by one of the following moieties:

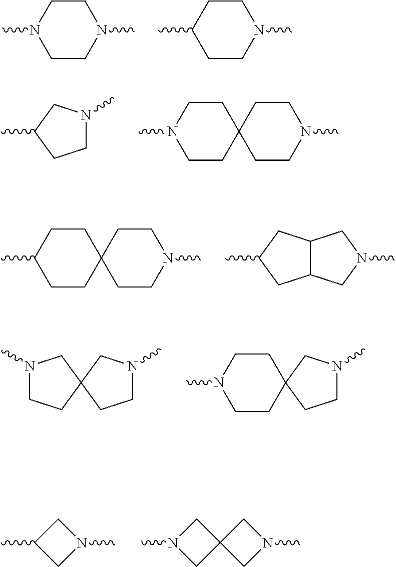

In another particular embodiment of the invention, the compound of formula (I) or (Ia) according to the invention is a compound wherein
A is an amine according to the following group:

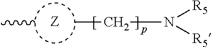

wherein
Z is a C₄₋₆-cycloalkyl or an N-containing heterocyclyl wherein said heterocyclyl is a saturated heterocyclyl;
p is 0, 1 or 2; preferably, p is 0 or 1; more preferably, p is 0;
R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

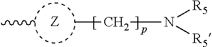

wherein
Z is a C₄₋₆-cycloalkyl;
p is 0 or 1; preferably p is 0;
R₅ is selected from the group consisting of substituted or unsubstituted C₁₋₆ alkyl and substituted or unsubstituted alkylaryl; and
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

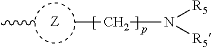

wherein
Z is a C₄₋₆-cycloalkyl;
p is 0;
R₅ is selected from the group consisting of substituted or unsubstituted C₁₋₆ alkyl and substituted or unsubstituted alkylaryl; preferably unsubstituted alkylaryl; more preferably, CH₂-phenyl; and
R₅′ is selected from the group consisting of hydrogen and unsubstituted C₁₋₃ alkyl, more preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

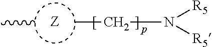

wherein
Z is a saturated N-containing heterocyclyl, wherein when said heterocyclyl is a polycyclic heterocyclyl then it can only contain one heteroatom per ring;
p is 0, 1 or 2; preferably, p is 0 or 1;
R₅ is selected from the group consisting of substituted or unsubstituted C₁₋₆ alkyl, substituted or unsubstituted alkylaryl; preferably, substituted or unsubstituted C₁₋₃ alkyl-phenyl, and substituted or unsubstituted alkylheterocyclyl; preferably —N-containing or O-containing heterocyclyl; and
R₅′ is selected from the group consisting of hydrogen and unsubstituted C₁₋₃ alkyl, more preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In more particular embodiment of the invention, the compound of formula (I) or (Ia) according to the invention is a compound wherein
A is an amine according to the following group:

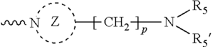

wherein
Z is an N-containing heterocyclyl wherein said heterocyclyl is a saturated heterocyclyl containing only one nitrogen as heteroatom;
p is 0 or 1;
R₅ is selected from the group consisting of substituted or unsubstituted C₁₋₆ alkyl, substituted or unsubstituted alkylaryl; preferably, substituted or unsubstituted C₁₋₃ alkyl-phenyl, and substituted or unsubstituted alkylheterocyclyl; preferably C₁₋₃ alkyl-N-containing heterocyclyl or C₁₋₃ alkyl-O-containing heterocyclyl; and
R₅′ is selected from the group consisting of hydrogen and unsubstituted C₁₋₃ alkyl, more preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

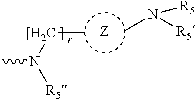

wherein
r is 0, 1 or 2; preferably r is 0 or 1;
Z is a C₄₋₆-cycloalkyl;
R₅″ is hydrogen or substituted or unsubstituted C₁₋₆ alkyl;
R₅ is selected from the group consisting of substituted or unsubstituted C₁₋₆ alkyl and substituted or unsubstituted alkylaryl; and
R₅′ is selected from the group consisting of hydrogen and substituted or unsubstituted C₁₋₆ alkyl, preferably unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) or (Ia) according to the invention is a compound wherein
A is an amine according to the following group:

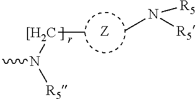

wherein
r is 0 or 1;
Z is a C₄₋₆-cycloalkyl;
R₅″ is hydrogen or substituted or unsubstituted C₁₋₆ alkyl, preferably unsubstituted C₁₋₆ alkyl, more preferably, methyl;
R₅ is selected from the group consisting of substituted or unsubstituted C₁₋₆ alkyl and substituted or unsubstituted alkylaryl, preferably C₁₋₃ alkyl-phenyl, more preferably, benzyl; and
R₅′ is unsubstituted C₁₋₆ alkyl, more preferably, methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular and preferred embodiment of the invention Z is represented in the compound of formula (I) or (Ia) by one of the following moieties:

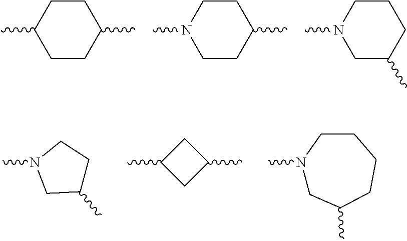

In another preferred embodiment of the invention according to formula (I) the compound is a compound, wherein in R₁, R₂, R₅′, R₅″ and R₅′″ as defined in any of the embodiments of the present invention,
the C₁₋₆ alkyl is preferably selected from methyl, ethyl, propyl, butyl, pentyl, hexyl, isopropyl, and 2-methylpropyl;
and/or
the C₂₋₆-alkenyl is preferably selected from ethylene, propylene, butylene, pentylene, hexylene, isopropylene, and isobutylene;
and/or
the C₂₋₆-alkynyl is preferably selected from ethyne, propyne, butyne, pentyne, hexyne, isopropyne, and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₃ and R₄ as defined in any of the embodiments of the present invention,
the C₁₋₆ alkyl is preferably selected from methyl, ethyl, propyl, butyl, pentyl, hexyl, isopropyl, and 2-methylpropyl;
and/or
the C₂₋₆-alkenyl is preferably selected from ethylene, propylene, butylene, pentylene, hexylene, isopropylene, and isobutylene;
and/or
the C₂₋₆-alkynyl is preferably selected from ethyne, propyne, butyne, pentyne, hexyne, isopropyne, and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₅ as defined in any of the embodiments of the present invention,
the C₁₋₆ alkyl is preferably selected from methyl, ethyl, propyl, butyl, pentyl, hexyl, isopropyl, isopentyl and 2-methylpropyl;
and/or
the C₂₋₆-alkenyl is preferably selected from ethylene, propylene, butylene, pentylene, hexylene, isopropylene, and isobutylene;
and/or
the C₂₋₆-alkynyl is preferably selected from ethyne, propyne, butyne, pentyne, hexyne, isopropyne, and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from oxetane, azetidine, oxazepane, pyrrolidine, imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyran, tetrahydrofuran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole, and quinazoline; more preferably is pyridine or tetrahydropyran;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) according to the invention is a compound wherein
m is 0, 1 or 2; preferably, m is 0 or 1;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) according to the invention is a compound wherein
n is 0, 1 or 2; preferably, n is 0 or 1;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) according to the invention is a compound wherein
p is 0, 1 or 2; preferably, p is 0 or 1;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) according to the invention is a compound wherein
q is 0, 1 or 2; preferably, q is 0 or 1;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another particular embodiment of the invention, the compound of formula (I) according to the invention is a compound wherein
r is 0, 1 or 2; preferably, r is 0 or 1;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the compound according to the invention of formula (I) the halogen is fluorine, bromine or chlorine; preferably, the halogen is fluorine or chlorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a particular embodiment of the invention, the alkyl, alkenyl or alkynyl as defined in R₁-R₅^{iv}, if substituted, is substituted with one or more substituent/s selected from —OR′, halogen, —CN, haloalkyl, haloalkoxy and —NR′R″; each of the R′ and R″ groups is independently selected from the group consisting of hydrogen and unsubstituted C₁₋₆ alkyl, preferably methyl.

In a preferred embodiment of the compound according to the invention of formula (I) the alkyl, as defined in R₁, if substituted, is substituted with halogen, preferably fluorine.

In a preferred embodiment of the compound according to the invention of formula (I), the alkyl, as defined in R₅, if substituted, is substituted with one or more substituent/s selected from halogen, unsubstituted C₁₋₆ alkyl and —OR′; wherein R′ is hydrogen or unsubstituted C₁₋₆ alkyl, preferably methyl.

In another preferred embodiment of the compound according to the invention of formula (I), the alkylaryl, in particular, the benzyl, as defined in R₅, if substituted, is substituted with one or more substituent/s selected from the group consisting of halogen, —CN, SO₂R′, OR′, NR′R″, and CONR′R″; wherein each of the R′ and R″ groups is independently selected from the group consisting of hydrogen and unsubstituted C₁₋₆ alkyl, or R′ and R″ together with the N form a cycle.

In a preferred embodiment, the compound of the invention according to formula (I) is a compound, wherein
R₁ is selected from the group consisting of hydrogen and unsubstituted or substituted C₁₋₆ alkyl;
and/or
R₂ is selected from the group consisting of hydrogen and unsubstituted or substituted C₁₋₆ alkyl;
and/or
R₃ is selected from the group consisting of hydrogen, halogen, unsubstituted or substituted C₁₋₆ alkyl, CN and OR₃′;
   wherein R₃′ is unsubstituted or substituted C₁₋₆ alkyl;
and/or
R₄ is selected from the group consisting of hydrogen, halogen, unsubstituted or substituted C₁₋₆ alkyl and CN;
and/or
A is a linear or cyclic amine selected from one of the following groups:

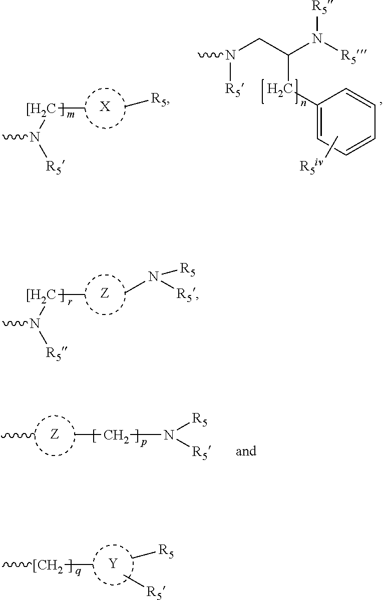

wherein
X is a N-containing heterocyclyl wherein said heterocyclyl is a saturated heterocyclyl;
and/or
Y is a N-containing heterocyclyl, wherein said heterocyclyl is a saturated heterocyclyl containing 1 to 2 nitrogen atoms;
and/or
Z is a C₄₋₆-cycloalkyl or an N-containing heterocyclyl wherein said heterocyclyl is a saturated heterocyclyl;
and/or
m is 0, 1 or 2;
and/or
n is 0, 1 or 2;
and/or
p is 0, 1 or 2;
and/or
q is 0, 1 or 2;
and/or
r is 0, 1 or 2;
and/or
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and/or
   R₅′ is selected from the group consisting of hydrogen and a non-substituted C₁₋₆ alkyl;
   and/or
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
   alternatively, R₅″ and R₅′″ may form, together with the nitrogen atom to which they are attached, a substituted or unsubstituted N-containing heterocyclyl;
   and/or
   R₅^{iv} is selected from the group consisting of hydrogen, halogen and OR₆; wherein
      R₆ is substituted or unsubstituted alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) is a compound of formula (Ia):

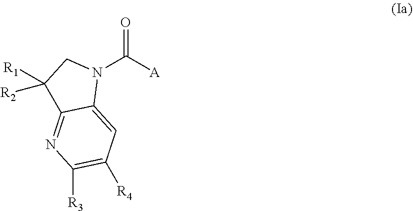

R₁ is selected from the group consisting of hydrogen and unsubstituted or substituted C₁₋₆ alkyl;
and/or
R₂ is selected from the group consisting of hydrogen and unsubstituted or substituted C₁₋₆ alkyl;
and/or
R₃ is selected from the group consisting of hydrogen, halogen, unsubstituted or substituted C₁₋₆ alkyl, CN and OR₃′;
   wherein R₃′ is unsubstituted or substituted C₁₋₆ alkyl;
and/or
R₄ is selected from the group consisting of hydrogen, halogen, unsubstituted or substituted C₁₋₆ alkyl and CN;
and/or
A is a linear or cyclic amine selected from one of the following groups:

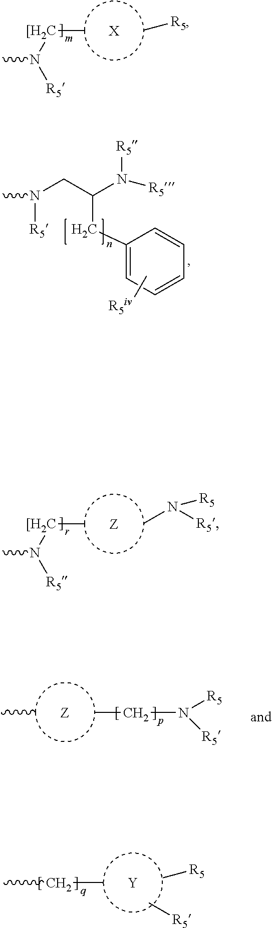

wherein
X is a N-containing heterocyclyl wherein said heterocyclyl is a saturated heterocyclyl;
and/or
Y is a N-containing heterocyclyl, wherein said heterocyclyl is a saturated heterocyclyl containing 1 to 2 nitrogen atoms;
and/or
Z is a C₄₋₆-cycloalkyl or an N-containing heterocyclyl wherein said heterocyclyl is a saturated heterocyclyl;
and/or
m is 0, 1 or 2;
and/or
n is 0, 1 or 2;
and/or
p is 0, 1 or 2;
and/or
q is 0, 1 or 2;
and/or
r is 0, 1 or 2;
and/or
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and/or
   R₅′ is selected from the group consisting of hydrogen and a non-substituted C₁₋₆ alkyl;
   and/or
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
   alternatively, R₅″ and R₅′″ may form, together with the nitrogen atom to which they are attached, a substituted or unsubstituted N-containing heterocyclyl;
   and/or
   R₅^{iv} is selected from the group consisting of hydrogen, halogen and OR₆; wherein
      R₆ is substituted or unsubstituted alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is a linear or cyclic amine selected from one of the following groups:

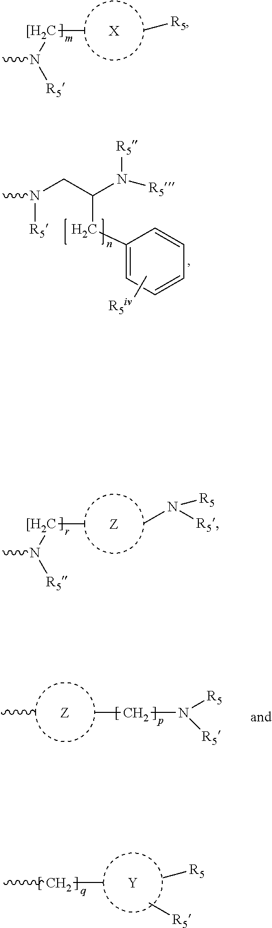

wherein
X is a N-containing heterocyclyl wherein said heterocyclyl is a saturated heterocyclyl;
and/or
Y is a N-containing heterocyclyl, wherein said heterocyclyl is a saturated heterocyclyl containing 1 to 2 nitrogen atoms;
and/or
Z is a C4-6-cycloalkyl or an N-containing heterocyclyl wherein said heterocyclyl is a
saturated heterocyclyl;
and/or
m is 0, 1 or 2;
and/or
n is 0, 1 or 2;
and/or
p is 0, 1 or 2;
and/or
q is 0, 1 or 2;
and/or
r is 0, 1 or 2;
and/or
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and/or
   R₅′ is selected from the group consisting of hydrogen and a non-substituted C₁₋₆ alkyl;
   and/or
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
   alternatively, R₅″ and R₅′″ may form, together with the nitrogen atom to which they are attached, a substituted or unsubstituted N-containing heterocyclyl;
   and/or
   R₅^{iv} is selected from the group consisting of hydrogen, halogen and OR₆; wherein
      R₆ is substituted or unsubstituted alkyl;

wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is an amine according to the following group:

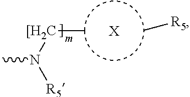

wherein
X is a N-containing heterocyclyl, wherein said heterocyclyl is a saturated heterocyclyl;
and/or
m is 0, 1, or 2; preferably m is 0 or 1;
and/or
R₅ is selected from the group consisting of substituted or unsubstituted C₁₋₆ alkyl, substituted or unsubstituted alkylaryl, unsubstituted heterocyclyl, and substituted or unsubstituted alkylheterocyclyl; and
R₅′ is hydrogen;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is a linear amine according to the following group:

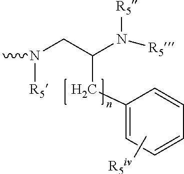

wherein:
n is 0 or 1;
and/or
R₅″ and R₅′″ are independently selected from the group consisting of hydrogen and substituted or unsubstituted C₁₋₆ alkyl; preferably unsubstituted C₁₋₆ alkyl, more preferably, unsubstituted C₁₋₃ alkyl;
   alternatively, R₅″ and R₅′″ may form, together with the nitrogen atom to which they are attached, a substituted or unsubstituted N-containing heterocyclyl;
and/or
R₅^{iv} is selected from the group consisting of hydrogen, halogen, preferably fluorine, and OR₆; wherein
   R₆ is substituted or unsubstituted alkyl, preferably unsubstituted C₁₋₃ alkyl, more preferably methyl;
and/or
R₅′ is selected from the group consisting of hydrogen and a non-substituted C₁₋₆ alkyl, preferably methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl, more preferably, methyl;
and/or
R₄ is selected from the group consisting of hydrogen, halogen, unsubstituted or substituted C₁₋₆ alkyl and CN;
and
A is an amine according to the following group:

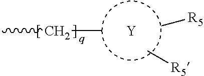

wherein
q is 0, 1 or 2; preferably, q is 0 or 1;
and/or
Y is a N-containing heterocyclyl, wherein said heterocyclyl is a saturated heterocyclyl containing 1 to 2 nitrogen atoms;
and/or
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
and/or
R₅′ is selected from the group consisting of hydrogen and a non-substituted C₁₋₆ alkyl, preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an even more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is an amine according to the following group:

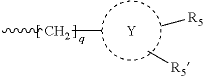

wherein
q is 0 or 1;
and/or
Y is a N-containing heterocyclyl, wherein said heterocyclyl is a mono or bicyclic saturated heterocyclyl containing 1 to 2 nitrogen atoms; wherein when said heterocyclyl is a bicyclic heterocyclyl then it contains one nitrogen atom per ring;
and/or
R₅ is selected from the group consisting of substituted or unsubstituted C₁₋₆ alkyl, substituted or unsubstituted alkylaryl, preferably CH₂-phenyl, substituted or unsubstituted alkylcycloalkyl, substituted or unsubstituted heterocyclyl, preferably unsubstituted O-containing heterocyclyl, and substituted or unsubstituted alkylheterocyclyl, preferably N-containing or O-containing heterocyclyl;
and/or
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is an amine according to the following group:

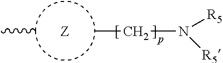

wherein
Z is a C4-6-cycloalkyl or an N-containing heterocyclyl wherein said heterocyclyl is a saturated heterocyclyl;
and/or
p is 0, 1 or 2; preferably, p is 0 or 1; more preferably, p is 0;
and/or
R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
and/or
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl, more preferably, methyl;
and/or
R₄ is selected from the group consisting of hydrogen, halogen, unsubstituted or substituted C₁₋₆ alkyl and CN;
and/or
A is an amine according to the following group:

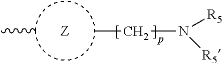

wherein
Z is a C₄₋₆-cycloalkyl;
and/or
p is 0 or 1, preferably p is 0;
and/or
R₅ is selected from the group consisting of substituted or unsubstituted C₁₋₆ alkyl and substituted or unsubstituted alkylaryl;
and/or
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and/or
R₄ is selected from the group consisting of hydrogen, halogen, unsubstituted or substituted C₁₋₆ alkyl and CN;
and/or
A is an amine according to the following group:

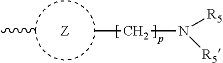

wherein
Z is a saturated N-containing heterocyclyl, wherein when said heterocyclyl is a polycyclic heterocyclyl then it can only contain one heteroatom per ring;
and/or
p is 0, 1 or 2; preferably, p is 0 or 1;
and/or
R₅ is selected from the group consisting of substituted or unsubstituted C₁₋₆ alkyl, substituted or unsubstituted alkylaryl; preferably, substituted or unsubstituted C₁₋₃ alkyl-phenyl, and substituted or unsubstituted alkylheterocyclyl; preferably —N-containing or O-containing heterocyclyl;
and/or
R₅′ is selected from the group consisting of hydrogen and unsubstituted C₁₋₃ alkyl, more preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and/or
R₂ is selected from the group consisting of hydrogen and unsubstituted or substituted C₁₋₆ alkyl, preferably ethyl or methyl; more preferably, methyl;
and/or
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl, more preferably, methyl;
and/or
R₄ is selected from the group consisting of hydrogen, halogen, unsubstituted or substituted C₁₋₆ alkyl and CN;
and/or
A is an amine according to the following group:

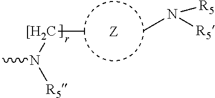

wherein
r is 0, 1 or 2, preferably r is 0 or 1;
and/or
Z is a C₄₋₆-cycloalkyl;
and/or
R₅″ is hydrogen or substituted or unsubstituted C₁₋₆ alkyl;
and/or
R₅ is selected from the group consisting of substituted or unsubstituted C₁₋₆ alkyl and substituted or unsubstituted alkylaryl;
and/or
R₅′ is selected from the group consisting of hydrogen and substituted or unsubstituted C₁₋₆ alkyl, preferably unsubstituted C₁₋₆ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compound of the invention according to formula (I) is a compound, wherein
R₁ is selected from the group consisting of hydrogen and unsubstituted or substituted C₁₋₆ alkyl;
and
R₂ is selected from the group consisting of hydrogen and unsubstituted or substituted C₁₋₆ alkyl;
and
R₃ is selected from the group consisting of hydrogen, halogen, unsubstituted or substituted C₁₋₆ alkyl, CN and OR₃′;
   wherein R₃′ is unsubstituted or substituted C₁₋₆ alkyl;
and
R₄ is selected from the group consisting of hydrogen, halogen, unsubstituted or substituted C₁₋₆ alkyl and CN;
and
A is a linear or cyclic amine selected from one of the following groups:

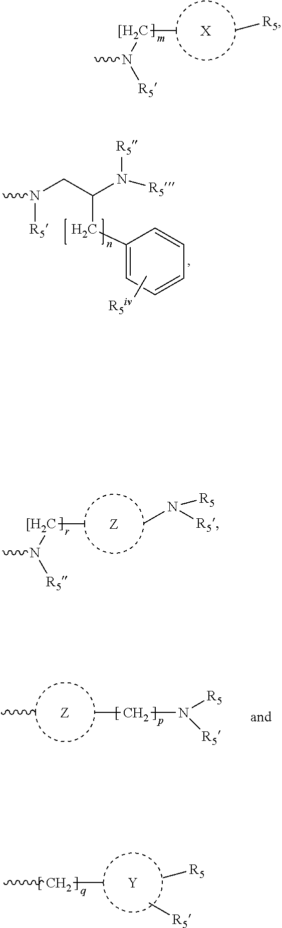

wherein
X is a N-containing heterocyclyl wherein said heterocyclyl is a saturated heterocyclyl;
and
Y is a N-containing heterocyclyl, wherein said heterocyclyl is a saturated heterocyclyl containing 1 to 2 nitrogen atoms;
and
Z is a C₄₋₆-cycloalkyl or an N-containing heterocyclyl wherein said heterocyclyl is a saturated heterocyclyl;
and
m is 0, 1 or 2;
and
n is 0, 1 or 2;
and
p is 0, 1 or 2;
and
q is 0, 1 or 2;
and
r is 0, 1 or 2;
and
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and
   R₅′ is selected from the group consisting of hydrogen and a non-substituted C₁₋₆ alkyl;
   and
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆alkynyl;
   alternatively, R₅″ and R₅′″ may form, together with the nitrogen atom to which they are attached, a substituted or unsubstituted N-containing heterocyclyl;
   and
   R₅^{iv} is selected from the group consisting of hydrogen, halogen and OR₆; wherein
      R₆ is substituted or unsubstituted alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) is a compound of formula (Ia):

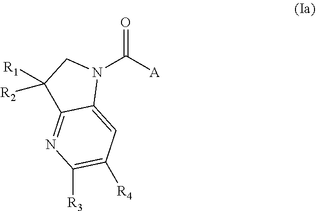

R₁ is selected from the group consisting of hydrogen and unsubstituted or substituted C₁₋₆ alkyl;
and
R₂ is selected from the group consisting of hydrogen and unsubstituted or substituted C₁₋₆ alkyl;
and
R₃ is selected from the group consisting of hydrogen, halogen, unsubstituted or substituted C₁₋₆ alkyl, CN and OR₃′;
   wherein R₃′ is unsubstituted or substituted C₁₋₆ alkyl;
and
R₄ is selected from the group consisting of hydrogen, halogen, unsubstituted or substituted C₁₋₆ alkyl and CN;
and
A is a linear or cyclic amine selected from one of the following groups:

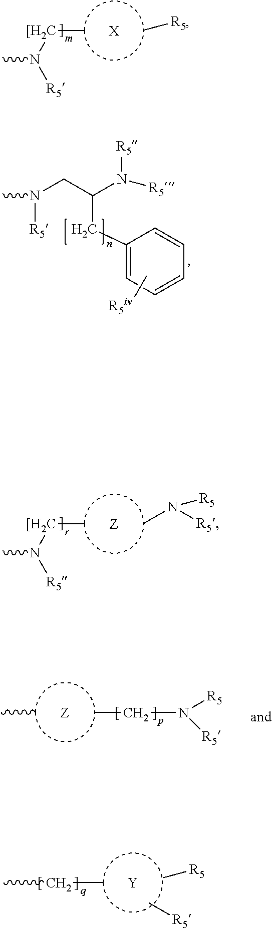

wherein
X is a N-containing heterocyclyl wherein said heterocyclyl is a saturated heterocyclyl;
and
Y is a N-containing heterocyclyl, wherein said heterocyclyl is a saturated heterocyclyl containing 1 to 2 nitrogen atoms;
and
Z is a C₄₋₆-cycloalkyl or an N-containing heterocyclyl wherein said heterocyclyl is a saturated heterocyclyl;
and
m is 0, 1 or 2;
and
n is 0, 1 or 2;
and
p is 0, 1 or 2;
and
q is 0, 1 or 2;
and
r is 0, 1 or 2;
and
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and
   R₅′ is selected from the group consisting of hydrogen and a non-substituted C₁₋₆ alkyl;
   and
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆alkynyl;
   alternatively, R₅″ and R₅′″ may form, together with the nitrogen atom to which they are attached, a substituted or unsubstituted N-containing heterocyclyl;
   and
   R₅^{iv} is selected from the group consisting of hydrogen, halogen and OR₆; wherein
      R₆ is substituted or unsubstituted alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is a linear or cyclic amine selected from one of the following groups:

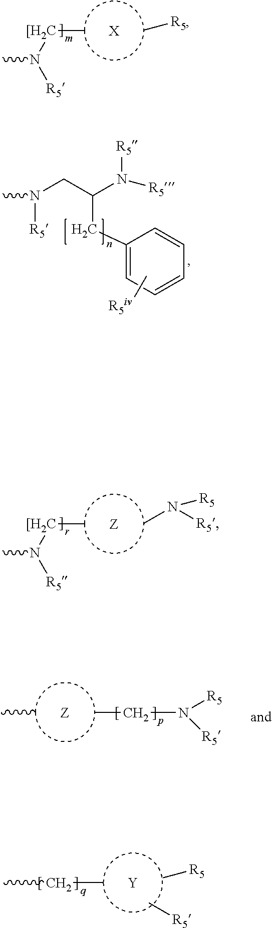

wherein
X is a N-containing heterocyclyl wherein said heterocyclyl is a saturated heterocyclyl;
and
Y is a N-containing heterocyclyl, wherein said heterocyclyl is a saturated heterocyclyl containing 1 to 2 nitrogen atoms;
and
Z is a C₄₋₆-cycloalkyl or an N-containing heterocyclyl wherein said heterocyclyl is a saturated heterocyclyl;
and
m is 0, 1 or 2;
and
n is 0, 1 or 2;
and
p is 0, 1 or 2;
and
q is 0, 1 or 2;
and
r is 0, 1 or 2;
and
   R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
   and
   R₅′ is selected from the group consisting of hydrogen and a non-substituted C₁₋₆ alkyl;
   and
   R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆alkynyl;
   alternatively, R₅″ and R₅′″ may form, together with the nitrogen atom to which they are attached, a substituted or unsubstituted N-containing heterocyclyl;
   and
   R₅^{iv} is selected from the group consisting of hydrogen, halogen and OR₆; wherein
      R₆ is substituted or unsubstituted alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

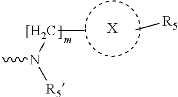

wherein
X is a N-containing heterocyclyl, wherein said heterocyclyl is a saturated heterocyclyl;
and
m is 0, 1, or 2; preferably m is 0 or 1;
and
R₅ is selected from the group consisting of substituted or unsubstituted C₁₋₆ alkyl, substituted or unsubstituted alkylaryl, unsubstituted heterocyclyl, and substituted or unsubstituted alkylheterocyclyl; and
R₅′ is hydrogen;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆alkyl, preferably ethyl or methyl, more preferably, methyl;
and
R₂ is selected from the group consisting of hydrogen and unsubstituted or substituted C₁₋₆alkyl, preferably ethyl or methyl, more preferably, methyl;
and
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆alkyl, more preferably, methyl;
and
R₄ is selected from the group consisting of hydrogen, halogen, unsubstituted or substituted C₁₋₆ alkyl and CN;
and
A is a linear amine according to the following group:

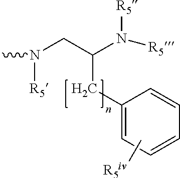

wherein:
n is 0 or 1;
and
R₅″ and R₅′″ are independently selected from the group consisting of hydrogen and substituted or unsubstituted C₁₋₆ alkyl; preferably unsubstituted C₁₋₆ alkyl, more preferably, unsubstituted C₁₋₃ alkyl;
   alternatively, R₅″ and R₅′″ may form, together with the nitrogen atom to which they are attached, a substituted or unsubstituted N-containing heterocyclyl;
and
R₅^{iv} is selected from the group consisting of hydrogen, halogen, preferably fluorine, and OR₆; wherein
   R₆ is substituted or unsubstituted alkyl, preferably unsubstituted C₁₋₃ alkyl, more preferably methyl;
and
R₅′ is selected from the group consisting of hydrogen and a non-substituted C₁₋₆ alkyl, preferably methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

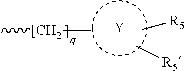

wherein
q is 0, 1 or 2; preferably, q is 0 or 1;
and
Y is a N-containing heterocyclyl, wherein said heterocyclyl is a saturated heterocyclyl containing 1 to 2 nitrogen atoms;
and
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
and
R₅′ is selected from the group consisting of hydrogen and a non-substituted C₁₋₆ alkyl, preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an even more preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

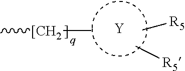

wherein
q is 0 or 1;
and
Y is a N-containing heterocyclyl, wherein said heterocyclyl is a mono or bicyclic saturated heterocyclyl containing 1 to 2 nitrogen atoms; wherein when said heterocyclyl is a bicyclic heterocyclyl then it contains one nitrogen atom per ring;
and
R₅ is selected from the group consisting of substituted or unsubstituted C₁₋₆ alkyl, substituted or unsubstituted alkylaryl, preferably CH₂-phenyl, substituted or unsubstituted alkylcycloalkyl, substituted or unsubstituted heterocyclyl, preferably unsubstituted O-containing heterocyclyl, and substituted or unsubstituted alkylheterocyclyl, preferably N-containing or O-containing heterocyclyl;
and
R₅′ is selected from the group consisting of hydrogen and methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

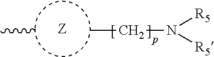

wherein
Z is a C₄₋₆-cycloalkyl or an N-containing heterocyclyl wherein said heterocyclyl is a saturated heterocyclyl;
and
p is 0, 1 or 2; preferably, p is 0 or 1; more preferably, p is 0;
and
R₅ is selected from the group consisting of substituted or unsubstituted C₁₋₆ alkyl, substituted or unsubstituted alkylaryl, substituted or unsubstituted cycloalkyl, substituted or unsubstituted alkylcycloalkyl, substituted or unsubstituted heterocyclyl, and substituted or unsubstituted alkylheterocyclyl;
and
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

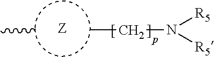

wherein
Z is a C₄₋₆-cycloalkyl;
and
p is 0 or 1, preferably p is 0;
and
R₅ is selected from the group consisting of substituted or unsubstituted C₁₋₆ alkyl and substituted or unsubstituted alkylaryl;
and
R₅′ is selected from the group consisting of hydrogen and substituted or unsubstituted C₁₋₆ alkyl, preferably unsubstituted C₁₋₆ alkyl, more preferably unsubstituted C₁₋₃ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl, more preferably, methyl;
and
R₂ is selected from the group consisting of hydrogen and unsubstituted or substituted C₁₋₆ alkyl, preferably ethyl or methyl, more preferably, methyl;
and
R₃ is selected from the group consisting of hydrogen, halogen, unsubstituted or substituted C₁₋₆ alkyl, preferably methyl, CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

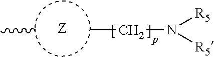

wherein
Z is a saturated N-containing heterocyclyl, wherein when said heterocyclyl is a polycyclic heterocyclyl then it can only contain one heteroatom per ring;
and
p is 0, 1 or 2; preferably, p is 0 or 1;
and
R₅ is selected from the group consisting of substituted or unsubstituted C₁₋₆ alkyl, substituted or unsubstituted alkylaryl; preferably, substituted or unsubstituted C₁₋₃ alkyl-phenyl, and substituted or unsubstituted alkylheterocyclyl; preferably —N-containing or O-containing heterocyclyl;
and
R₅′ is selected from the group consisting of hydrogen and unsubstituted C₁₋₃ alkyl, more preferably, methyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound of the invention according to formula (I) or (Ia) is a compound wherein
R₁ is selected from the group consisting of hydrogen and unsubstituted or substituted C₁₋₆ alkyl, preferably ethyl or methyl; more preferably, methyl;
and
R₂ is selected from the group consisting of hydrogen and unsubstituted or substituted C₁₋₆ alkyl, preferably ethyl or methyl; more preferably, methyl;
and
R₃ is selected from the group consisting of hydrogen, halogen, unsubstituted or substituted C₁₋₆ alkyl, preferably methyl; CN and OR₃′;
   wherein R₃′ is unsubstituted or substituted C₁₋₆ alkyl; preferably unsubstituted C₁₋₆ alkyl; more preferably, methyl;
and
R₄ is selected from the group consisting of hydrogen, halogen, unsubstituted or substituted C₁₋₆ alkyl and CN;
and
A is an amine according to the following group:

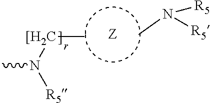

wherein
r is 0, 1 or 2; preferably r is 0 or 1;
and
Z is a C₄₋₆-cycloalkyl;
and
R₅″ is hydrogen or substituted or unsubstituted C₁₋₆ alkyl;
and
R₅ is selected from the group consisting of substituted or unsubstituted C₁₋₆ alkyl and substituted or unsubstituted alkylaryl;
and
R₅′ is selected from the group consisting of hydrogen and substituted or unsubstituted C₁₋₆ alkyl, preferably unsubstituted C₁₋₆ alkyl;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compound of formula (I) is selected from the group consisting of:
1 N-(1-benzylpiperidin-4-yl)-3,3,5-trimethyl-2,3-dihydro-1H-pyrrolo[3,2-b]pyridine-1-carboxamide;
2 N-(2-(dimethylamino)-2-phenylethyl)-3,3,5-trimethyl-2,3-dihydro-1H-pyrrolo[3,2-b]pyridine-1-carboxamide;
3 N-(2-(dimethylamino)-2-phenylethyl)-3,3-dimethyl-5-(trifluoromethyl)-2,3-dihydro-1H-pyrrolo[3,2-b]pyridine-1-carboxamide;
4 (R)—N-(2-(dimethylamino)-2-phenylethyl)-3,3,5-trimethyl-2,3-dihydro-1H-pyrrolo[3,2-b]pyridine-1-carboxamide;
5 (R)—N-(2-(dimethylamino)-2-phenylethyl)-3,3-dimethyl-2,3-dihydro-1H-pyrrolo[3,2-b]pyridine-1-carboxamide;
6 (R)—N-(2-(dimethylamino)-2-phenylethyl)-5-methyl-2,3-dihydro-1H-pyrrolo[3,2-t]pyridine-1-carboxamide;
7 3,3,5-trimethyl-N-(2-phenyl-2-(pyrrolidin-1-yl)ethyl)-2,3-dihydro-1H-pyrrolo[3,2-b]pyridine-1-carboxamide;
8 (R)—N-(2-(dimethylamino)-2-phenylethyl)-N,3,3,5-tetramethyl-2,3-dihydro-1H-pyrrolo[3,2-b]pyridine-1-carboxamide;
9 N-(2-(diethylamino)-2-phenylethyl)-3,3,5-trimethyl-2,3-dihydro-1H-pyrrolo[3,2-b]pyridine-1-carboxamide;
10 (4-benzylpiperazin-1-yl)(3,3,5-trimethyl-2,3-dihydro-1H-pyrrolo[3,2-b]pyridin-1-yl)methanone;
11 (4-(benzyl(methyl)amino)piperidin-1-yl)(3,3,5-trimethyl-2,3-dihydro-1H-pyrrolo[3,2-b]pyridin-1-yl)methanone
12 (S)—N-(2-(dimethylamino)-3-phenylpropyl)-3,3,5-trimethyl-2,3-dihydro-1H-pyrrolo[3,2-b]pyridine-1-carboxamide;
13 N-(2-(dimethylamino)-2-(4-methoxyphenyl)ethyl)-3,3,5-trimethyl-2,3-dihydro-1H-pyrrolo[3,2-b]pyridine-1-carboxamide;
14 (R)-6-chloro-N-(2-(dimethylamino)-2-phenylethyl)-3,3-dimethyl-2,3-dihydro-1H-pyrrolo[3,2-b]pyridine-1-carboxamide;
15 (R)-5-cyano-N-(2-(dimethylamino)-2-phenylethyl)-3,3-dimethyl-2,3-dihydro-1H-pyrrolo[3,2-b]pyridine-1-carboxamide;
16 (R)—N-(2-(dimethylamino)-2-phenylethyl)-6-fluoro-3,3,5-trimethyl-2,3-dihydro-1H-pyrrolo[3,2-b]pyridine-1-carboxamide;
17 (R)—N-(2-(dimethylamino)-2-phenylethyl)-5-methoxy-3,3-dimethyl-2,3-dihydro-1H-pyrrolo[3,2-b]pyridine-1-carboxamide;
18 (R)—N-(1-benzylpyrrolidin-3-yl)-3,3,5-trimethyl-2,3-dihydro-1H-pyrrolo[3,2-b]pyridine-1-carboxamide;
19 N-(2-(dimethylamino)-2-(2-fluorophenyl)ethyl)-3,3,5-trimethyl-2,3-dihydro-1H-pyrrolo[3,2-b]pyridine-1-carboxamide;
20 (R)—N-(2-(dimethylamino)-2-phenylethyl)-6-fluoro-3,3-dimethyl-2,3-dihydro-1H-pyrrolo[3,2-b]pyridine-1-carboxamide;
21 (S)—N-(1-benzylpyrrolidin-3-yl)-3,3,5-trimethyl-2,3-dihydro-1H-pyrrolo[3,2-b]pyridine-1-carboxamide;
22 N-(1-(4-fluorobenzyl)piperidin-4-yl)-3,3,5-trimethyl-2,3-dihydro-1H-pyrrolo[3,2-b]pyridine-1-carboxamide;
23 N-(1-(3-cyanobenzyl)piperidin-4-yl)-3,3,5-trimethyl-2,3-dihydro-1H-pyrrolo[3,2-b]pyridine-1-carboxamide;
24 N-(1-(4-cyanobenzyl)piperidin-4-yl)-3,3,5-trimethyl-2,3-dihydro-1H-pyrrolo[3,2-b]pyridine-1-carboxamide;
25 N-(1-isopentylpiperidin-4-yl)-3,3,5-trimethyl-2,3-dihydro-1H-pyrrolo[3,2-b]pyridine-1-carboxamide;
26 N-(1-(3-fluorobenzyl)piperidin-4-yl)-3,3,5-trimethyl-2,3-dihydro-1H-pyrrolo[3,2-b]pyridine-1-carboxamide;
27 3,3,5-trimethyl-N-(1-phenethylpiperidin-4-yl)-2,3-dihydro-1H-pyrrolo[3,2-b]pyridine-1-carboxamide;
28 N-(1-(2-ethoxyethyl)piperidin-4-yl)-3,3,5-trimethyl-2,3-dihydro-1H-pyrrolo[3,2-b]pyridine-1-carboxamide;
29 (4-(benzyl(methyl)amino)piperidin-1-yl)(3,3-dimethyl-2,3-dihydro-1H-pyrrolo[3,2-b]pyridin-1-yl)methanone;
30 N-(1-benzylpiperidin-4-yl)-5-cyano-3,3-dimethyl-2,3-dihydro-1H-pyrrolo[3,2-b]pyridine-1-carboxamide;
31 N-(1-benzylpiperidin-4-yl)-6-fluoro-3,3-dimethyl-2,3-dihydro-1H-pyrrolo[3,2-b]pyridine-1-carboxamide;
32 N-(1-benzylpiperidin-4-yl)-3,3-dimethyl-2,3-dihydro-1H-pyrrolo[3,2-b]pyridine-1-carboxamide;
33 (R)—N-(2-(dimethylamino)-2-(4-fluorophenyl)ethyl)-3,3,5-trimethyl-2,3-dihydro-1H-pyrrolo[3,2-b]pyridine-1-carboxamide;
34 (R)—N-(2-(dimethylamino)-2-(3-fluorophenyl)ethyl)-3,3,5-trimethyl-2,3-dihydro-1H-pyrrolo[3,2-b]pyridine-1-carboxamide;
35 (R)—N-(2-(dimethylamino)-2-(3-methoxyphenyl)ethyl)-3,3,5-trimethyl-2,3-dihydro-1H-pyrrolo[3,2-b]pyridine-1-carboxamide;
36 ((3aR,6aS)-5-benzylhexahydropyrrolo[3,4-c]pyrrol-2(1H)-yl)(3,3,5-trimethyl-2,3-dihydro-1H-pyrrolo[3,2-b]pyridin-1-yl)methanone;
37 (7-benzyl-2,7-diazaspiro[4.4]nonan-2-yl)(3,3,5-trimethyl-2,3-dihydro-1H-pyrrolo[3,2-b]pyridin-1-yl)methanone;
38 (2-benzyl-2,8-diazaspiro[4.5]decan-8-yl)(3,3,5-trimethyl-2,3-dihydro-1H-pyrrolo[3,2-b]pyridin-1-yl)methanone;
39 (S)-(3-(benzyl(methyl)amino)pyrrolidin-1-yl)(3,3,5-trimethyl-2,3-dihydro-1H-pyrrolo[3,2-b]pyridin-1-yl)methanone;
40 1-(4-(benzyl(methyl)amino)piperidine-1-carbonyl)-3,3-dimethyl-2,3-dihydro-1H-pyrrolo[3,2-b]pyridine-5-carbonitrile;
41 N-(1-isobutylpiperidin-4-yl)-3,3,5-trimethyl-2,3-dihydro-1H-pyrrolo[3,2-b]pyridine-1-carboxamide;
42 3,3,5-trimethyl-N-(1-((tetrahydro-2H-pyran-4-yl)methyl)piperidin-4-yl)-2,3-dihydro-1H-pyrrolo[3,2-b]pyridine-1-carboxamide;
43 (R)-(3-(benzyl(methyl)amino)pyrrolidin-1-yl)(3,3,5-trimethyl-2,3-dihydro-1H-pyrrolo[3,2-b]pyridin-1-yl)methanone;
44 N-(1-(3,4-difluorobenzyl)piperidin-4-yl)-3,3,5-trimethyl-2,3-dihydro-1H-pyrrolo[3,2-b]pyridine-1-carboxamide;
45 5-(((1-(3,3-dimethyl-2,3-dihydro-1H-pyrrolo[3,2-b]pyridine-1-carbonyl)piperidin-4-yl)(methyl)amino)methyl)-2-fluorobenzonitrile;
46 N-(1-(3,4-difluorobenzyl)piperidin-4-yl)-3,3-dimethyl-2,3-dihydro-1H-pyrrolo[3,2-b]pyridine-1-carboxamide;
47 N-(1-(3-fluorobenzyl)piperidin-4-yl)-3,3-dimethyl-2,3-dihydro-1H-pyrrolo[3,2-b]pyridine-1-carboxamide;
48 N-(1-(4-fluorobenzyl)piperidin-4-yl)-3,3-dimethyl-2,3-dihydro-1H-pyrrolo[3,2-b]pyridine-1-carboxamide;
49 (4-(benzyl(methyl)amino)piperidin-1-yl)(5-methyl-2,3-dihydro-1H-pyrrolo[3,2-b]pyridin-1-(4-(methyl(phenethyl)amino)piperidin-1-yl)(3,3,5-trimethyl-2,3-dihydro-1H-pyrrolo[3,2-b]pyridin-1-yl)methanone;
50 (4-(methyl(phenethyl)amino)piperidin-1-yl)(3,3,5-trimethyl-2,3-dihydro-1H-pyrrolo[3,2-b]pyridin-1-yl)methanone;
51 (4-(benzyl(methyl)amino)piperidin-1-yl)(2,3-dihydro-1H-pyrrolo[3,2-b]pyridin-1-yl)methanone;
52 (3,3-dimethyl-2,3-dihydro-1H-pyrrolo[3,2-b]pyridin-1-yl)(4-(methyl(phenethyl)amino)piperidin-1-yl)methanone;
53 (S)-(2,3-dihydro-1H-pyrrolo[3,2-b]pyridin-1-yl)(3-(methyl(phenethyl)amino)piperidin-1-yl)methanone;
54 (S)-(3-(methyl(phenethyl)amino)piperidin-1-yl)(3,3,5-trimethyl-2,3-dihydro-1H-pyrrolo[3,2-b]pyridin-1-yl)methanone;
55 N-(1-(3-chlorobenzyl)piperidin-4-yl)-3,3,5-trimethyl-2,3-dihydro-1H-pyrrolo[3,2-b]pyridine-1-carboxamide;
56 (4-(methyl(3-(methylsulfonyl)benzyl)amino)piperidin-1-yl)(3,3,5-trimethyl-2,3-dihydro-1H-pyrrolo[3,2-b]pyridin-1-yl)methanone;
57 (9-benzyl-3,9-diazaspiro[5.5]undecan-3-yl)(3,3,5-trimethyl-2,3-dihydro-1H-pyrrolo[3,2-b]pyridin-1-yl)methanone;
58 (4-((4-methoxybenzyl)(methyl)amino)piperidin-1-yl)(3,3,5-trimethyl-2,3-dihydro-1H-pyrrolo[3,2-b]pyridin-1-yl)methanone;
59 (44(3-methoxybenzyl)(methyl)amino)piperidin-1-yl)(3,3,5-trimethyl-2,3-dihydro-1H-pyrrolo[3,2-b]pyridin-1-yl)methanone;
60 2-fluoro-5-((methyl(1-(3,3,5-trimethyl-2,3-dihydro-1H-pyrrolo[3,2-b]pyridine-1-carbonyl)piperidin-4-yl)amino)methyl)benzonitrile;
61 (S)-(3-(benzyl(methy)amino)piperidin-1-yl)(2,3-dihydro-1H-pyrrolo[3,2-b]pyridin-1-yl)methanone;
62 (S)-(2,3-dihydro-1H-pyrrolo[3,2-b]pyridin-1-yl)(3-(methyl(phenethyl)amino)pyrrolidin-1-yl)methanone;
63 N, N-dimethyl-3-((methyl(1-(3,3,5-trimethyl-2,3-dihydro-1H-pyrrolo[3,2-b]pyridine-1-carbonyl)piperidin-4-yl)amino)methyl)benzamide;
64 (S)-(2,3-dihydro-1H-pyrrolo[3,2-b]pyridin-1-yl)(3-(isopentyl(methy)amino)piperidin-1-yl)methanone;
65 (4-(methyl((tetrahydro-2H-pyran-4-yl)methyl)amino)piperidin-1-yl)(3,3,5-trimethyl-2,3-dihydro-1H-pyrrolo[3,2-t]pyridin-1-yl)methanone;
66 (4-((benzyl(methyl)amino)methyl)piperidin-1-yl)(3,3,5-trimethyl-2,3-dihydro-1H-pyrrolo[3,2-b]pyridin-1-yl)methanone;
67 (4-(isopentyl(methyl)amino)piperidin-1-yl)(3,3,5-trimethyl-2,3-dihydro-1H-pyrrolo[3,2-b]pyridin-1-yl)methanone;
68 (4-((4-(dimethylamino)benzyl)(methyl)amino)piperidin-1-yl)(3,3,5-trimethyl-2,3-dihydro-1H-pyrrolo[3,2-b]pyridin-1-yl)methanone;
69 N-((1-benzylpiperidin-4-yl)methyl)-3,3-dimethyl-2,3-dihydro-1H-pyrrolo[3,2-b]pyridine-1-carboxamide;
70 3,3-dimethyl-N-((1-phenethylpiperidin-4-yl)methyl)-2,3-dihydro-1H-pyrrolo[3,2-b]pyridine-1-carboxamide;
71 N-((1r,4r)-4-(benzyl(methyl)amino)cyclohexyl)-3,3-dimethyl-2,3-dihydro-1H-pyrrolo[3,2-b]pyridine-1-carboxamide;
72 N-((1s,4s)-4-(benzyl(methyl)amino)cyclohexyl)-3,3-dimethyl-2,3-dihydro-1H-pyrrolo[3,2-b]pyridine-1-carboxamide;
73 (4-(methyl(pyridin-2-ylmethyl)amino)piperidin-1-yl)(3,3,5-trimethyl-2,3-dihydro-1H-pyrrolo[3,2-b]pyridin-1-yl)methanone;
74 (4-(methyl(pyridin-3-ylmethyl)amino)piperidin-1-yl)(3,3,5-trimethyl-2,3-dihydro-1H-pyrrolo[3,2-b]pyridin-1-yl)methanone;
75 (4-(methyl(pyridin-4-ylmethyl)amino)piperidin-1-yl)(3,3,5-trimethyl-2,3-dihydro-1H-pyrrolo[3,2-b]pyridin-1-yl)methanone;
76 3-(((1-(3,3-dimethyl-2,3-dihydro-1H-pyrrolo[3,2-b]pyridine-1-carbonyl)piperidin-4-yl)(methyl)amino)methyl)-5-fluorobenzonitrile;
77 3-(((1-(3,3-dimethyl-2,3-dihydro-1H-pyrrolo[3,2-b]pyridine-1-carbonyl)piperidin-4-yl)(methyl)amino)methyl)-4-fluorobenzonitrile;
78 N-((1r,4r)-4-(benzyl(methyl)amino)cyclohexyl)-N,3,3-trimethyl-2,3-dihydro-1H-pyrrolo[3,2-b]pyridine-1-carboxamide;
79 (4-((3,4-Difluorobenzyl)(methyl)amino)piperidin-1-yl)(3,3-dimethyl-2,3-dihydro-1H-pyrrolo[3,2-b]pyridin-1-yl)methanone;
80 (8-benzyl-2,8-diazaspiro[4.5]decan-2-yl)(3,3,5-trimethyl-2,3-dihydro-1H-pyrrolo[3,2-b]pyridin-1-yl)methanone;
81 (6-benzyl-2,6-diazaspiro[3.3]heptan-2-yl)(3,3,5-trimethyl-2,3-dihydro-1H-pyrrolo[3,2-b]pyridin-1-yl)methanone;
82 N-((1-benzylazetidin-3-yl)methyl)-3,3,5-trimethyl-2,3-dihydro-1H-pyrrolo[3,2-b]pyridine-1-carboxamide;
83 3-(((1-(3,3-dimethyl-2,3-dihydro-1H-pyrrolo[3,2-b]pyridine-1-carbonyl)piperidin-4-yl)(methyl)amino)methyl)benzonitrile;
84 4-(((1-(3,3-dimethyl-2,3-dihydro-1H-pyrrolo[3,2-b]pyridine-1-carbonyl)piperidin-4-yl)(methyl)amino)methyl)benzonitrile;
85 (3,3-dimethyl-2,3-dihydro-1H-pyrrolo[3,2-b]pyridin-1-yl)(4-((3-fluorobenzyl)(methyl)amino)piperidin-1-yl)methanone;
86 (3,3-dimethyl-2,3-dihydro-1H-pyrrolo[3,2-b]pyridin-1-yl)(4-((4-fluorobenzyl)(methyl)amino)piperidin-1-yl)methanone;
87 4-(((1-(3,3-dimethyl-2,3-dihydro-1H-pyrrolo[3,2-b]pyridine-1-carbonyl)piperidin-4-yl)(methyl)amino)methyl)-2-fluorobenzonitrile;
88 N-(1-benzylazetidin-3-yl)-3,3-dimethyl-2,3-dihydro-1H-pyrrolo[3,2-b]pyridine-1-carboxamide;
89 (8-benzyl-2,8-diazaspiro[4.5]decan-2-yl)(3,3-dimethyl-2,3-dihydro-1H-pyrrolo[3,2-b]pyridin-1-yl)methanone;
90 3-((2-(3,3-dimethyl-2,3-dihydro-1H-pyrrolo[3,2-b]pyridine-1-carbonyl)-2,8-diazaspiro[4.5]decan-8-yl)methyl)benzonitrile;
91 (8-phenethyl-2,8-diazaspiro[4.5]decan-2-yl)(3,3,5-trimethyl-2,3-dihydro-1H-pyrrolo[3,2-b]pyridin-1-yl)methanone;
92 3-((2-(3,3,5-trimethyl-2,3-dihydro-1H-pyrrolo[3,2-b]pyridine-1-carbonyl)-2,8-diazaspiro[4.5]decan-8-yl)methyl)benzonitrile;
93 (2-benzyl-2,7-diazaspiro[3.5]nonan-7-yl)(3,3,5-trimethyl-2,3-dihydro-1H-pyrrolo[3,2-b]pyridin-1-yl)methanone;
94 (8-(pyridin-2-ylmethyl)-2,8-diazaspiro[4.5]decan-2-yl)(3,3,5-trimethyl-2,3-dihydro-1H-pyrrolo[3,2-b]pyridin-1-yl)methanone;
95 (8-(3-methoxybenzyl)-2,8-diazaspiro[4.5]decan-2-yl)(3,3,5-trimethyl-2,3-dihydro-1H-pyrrolo[3,2-b]pyridin-1-yl)methanone;
96 (8-(1-phenylethyl)-2,8-diazaspiro[4.5]decan-2-yl)(3,3,5-trimethyl-2,3-dihydro-1H-pyrrolo[3,2-b]pyridin-1-yl)methanone;
97 (S)-(2,3-dihydro-1H-pyrrolo[3,2-b]pyridin-1-yl)(3-(phenethylamino)pyrrolidin-1-yl)methanone;
98 (8-(pyridin-3-ylmethyl)-2,8-diazaspiro[4.5]decan-2-yl)(3,3,5-trimethyl-2,3-dihydro-1H-pyrrolo[3,2-b]pyridin-1-yl)methanone;
99 (8-(pyridin-4-ylmethyl)-2,8-diazaspiro[4.5]decan-2-yl)(3,3,5-trimethyl-2,3-dihydro-1H-pyrrolo[3,2-b]pyridin-1-yl)methanone;
100 (8-isopentyl-2,8-diazaspiro[4.5]decan-2-yl)(3,3,5-trimethyl-2,3-dihydro-1H-pyrrolo[3,2-b]pyridin-1-yl)methanone;
101 (S)-(2,3-dihydro-1H-pyrrolo[3,2-b]pyridin-1-yl)(3-(phenethylamino)piperidin-1-yl)methanone;
102 (8-(3-(methylsulfonyl)benzyl)-2,8-diazaspiro[4.5]decan-2-yl)(3,3,5-trimethyl-2,3-dihydro-1H-pyrrolo[3,2-b]pyridin-1-yl)methanone;
103 (8-(4-methoxybenzyl)-2,8-diazaspiro[4.5]decan-2-yl)(3,3,5-trimethyl-2,3-dihydro-1H-pyrrolo[3,2-b]pyridin-1-yl)methanone;
104 N-(7-benzyl-7-azaspiro[3.5]nonan-2-yl)-3,3-dimethyl-2,3-dihydro-1H-pyrrolo[3,2-b]pyridine-1-carboxamide;
105 (8-((tetrahydro-2H-pyran-4-yl)methyl)-2,8-diazaspiro[4.5]decan-2-yl)(3,3,5-trimethyl-2,3-dihydro-1H-pyrrolo[3,2-t]pyridin-1-yl)methanone;
106 N-((1-isopentylpiperidin-4-yl)methyl)-3,3-dimethyl-2,3-dihydro-1H-pyrrolo[3,2-b]pyridine-1-carboxamide;
107 2-fluoro-5-((2-(3,3,5-trimethyl-2,3-dihydro-1H-pyrrolo[3,2-b]pyridine-1-carbonyl)-2,8-diazaspiro[4.5]decan-8-yl)methyl)benzonitrile;
108 (8-(2-(tetrahydro-2H-pyran-4-yl)ethyl)-2,8-diazaspiro[4.5]decan-2-yl)(3,3,5-trimethyl-2,3-dihydro-1H-pyrrolo[3,2-t]pyridin-1-yl)methanone;
109 N-((1-(3,3-dimethylbutyl)piperidin-4-yl)methyl)-3,3-dimethyl-2,3-dihydro-1H-pyrrolo[3,2-b]pyridine-1-carboxamide;
110 (8-(tetrahydro-2H-pyran-4-yl)-2,8-diazaspiro[4.5]decan-2-yl)(3,3,5-trimethyl-2,3-dihydro-1H-pyrrolo[3,2-b]pyridin-1-yl)methanone;
111 (S)-3,3,5-trimethyl-N-(2-(methylamino)-2-phenylethyl)-2,3-dihydro-1H-pyrrolo[3,2-b]pyridine-1-carboxamide;
112 (R)-3,3,5-trimethyl-N-(2-(methylamino)-2-phenylethyl)-2,3-dihydro-1H-pyrrolo[3,2-b]pyridine-1-carboxamide;
113 (R)—N-(2-(ethylamino)-2-phenylethyl)-3,3,5-trimethyl-2,3-dihydro-1H-pyrrolo[3,2-b]pyridine-1-carboxamide;
114 (4-((4-fluorobenzyl)(methyl)amino)piperidin-1-yl)(3,3,5-trimethyl-2,3-dihydro-1H-pyrrolo[3,2-b]pyridin-1-yl)methanone;
115 (4-(benzylamino)piperidin-1-yl)(3,3,5-trimethyl-2,3-dihydro-1H-pyrrolo[3,2-b]pyridin-1-yl)methanone;
116 (4-((3-fluorobenzyl)(methyl)amino)piperidin-1-yl)(3,3,5-trimethyl-2,3-dihydro-1H-pyrrolo[3,2-b]pyridin-1-yl)methanone;
117 4-((methyl(1-(3,3,5-trimethyl-2,3-dihydro-1H-pyrrolo[3,2-t]pyridine-1-carbonyl)piperidin-4-yl)amino)methyl)benzonitrile;
118 3-((methyl(1-(3,3,5-trimethyl-2,3-dihydro-1H-pyrrolo[3,2-t]pyridine-1-carbonyl)piperidin-4-yl)amino)methyl)benzonitrile;
119 (4-(isobutyl(methyl)amino)piperidin-1-yl)(3,3,5-trimethyl-2,3-dihydro-1H-pyrrolo[3,2-b]pyridin-1-yl)methanone
120 (3-(Isopentylamino)azepan-1-yl)(3,3,5-trimethyl-2,3-dihydro-1H-pyrrolo[3,2-t]pyridin-1-yl)methanone;
121 (S)-(2,3-dihydro-1H-pyrrolo[3,2-b]pyridin-1-yl)(3-(isopentylamino)azepan-1-yl)methanone;
122 (S)-(2,3-dihydro-1H-pyrrolo[3,2-b]pyridin-1-yl)(3-(isopentyl(methyl)amino)azepan-1-yl)methanone;
123 (1-benzylpiperidin-4-yl)(3,3,5-trimethyl-2,3-dihydro-1H-pyrrolo[3,2-b]pyridin-1-yl)methanone;
124 ((3S,4S)-1-benzyl-4-methylpyrrolidin-3-yl)(3,3,5-trimethyl-2,3-dihydro-1H-pyrrolo[3,2-b]pyridin-1-yl)methanone;
125 ((3R,4R)-1-benzyl-4-methylpyrrolidin-3-yl)(3,3,5-trimethyl-2,3-dihydro-1H-pyrrolo[3,2-b]pyridin-1-yl)methanone;
126 ((1s,4s)-4-(benzyl(methyl)amino)cyclohexyl)(3,3,5-trimethyl-2,3-dihydro-1H-pyrrolo[3,2-b]pyridin-1-yl)methanone;
127 ((1s,4s)-4-(benzyl(methyl)amino)cyclohexyl)(3,3-dimethyl-2,3-dihydro-1H-pyrrolo[3,2-b]pyridin-1-yl)methanone;
128 ((1r,4r)-4-(benzylamino)cyclohexyl)(3,3-dimethyl-2,3-dihydro-1H-pyrrolo[3,2-b]pyridin-1-yl)methanone dihydrochloride;
129 ((1r,4r)-4-(benzylamino)cyclohexyl)(3,3,5-trimethyl-2,3-dihydro-1H-pyrrolo[3,2-b]pyridin-1-yl)methanone dihydrochloride;
130 2-(1-benzylpiperidin-4-yl)-1-(3,3,5-trimethyl-2,3-dihydro-1H-pyrrolo[3,2-b]pyridin-1-yl)ethanone;
131 (1-benzylazetidin-3-yl)(3,3,5-trimethyl-2,3-dihydro-1H-pyrrolo[3,2-b]pyridin-1-yl)methanone;
132 (1-benzylazetidin-3-yl)(3,3-dimethyl-2,3-dihydro-1H-pyrrolo[3,2-b]pyridin-1-yl)methanone;
133 (1-(4-fluorobenzyl)azetidin-3-yl)(3,3,5-trimethyl-2,3-dihydro-1H-pyrrolo[3,2-b]pyridin-1-yl)methanone;
134 ((1r,3r)-3-(benzylamino)cyclobutyl)(3,3,5-trimethyl-2,3-dihydro-1H-pyrrolo[3,2-b]pyridin-1-yl)methanone;
135 (1-(3-fluorobenzyl)azetidin-3-yl)(3,3,5-trimethyl-2,3-dihydro-1H-pyrrolo[3,2-b]pyridin-1-yl)methanone;
136 ((1s,3s)-3-(benzylamino)cyclobutyl)(3,3,5-trimethyl-2,3-dihydro-1H-pyrrolo[3,2-b]pyridin-1-yl)methanone;
137 ((1r,3r)-3-(Benzyl(methyl)amino)cyclobutyl)(3,3,5-trimethyl-2,3-dihydro-1H-pyrrolo[3,2-b]pyridin-1-yl)methanone;
138 ((1r,4r)-4-(benzyl(methyl)amino)cyclohexyl)(3,3-dimethyl-2,3-dihydro-1H-pyrrolo[3,2-b]pyridin-1-yl)methanone;
139 ((1s,3s)-3-(benzyl(methyl)amino)cyclobutyl)(3,3,5-trimethyl-2,3-dihydro-1H-pyrrolo[3,2-b]pyridin-1-yl)methanone;
140 ((1r,4r)-4-(benzyl(methyl)amino)cyclohexyl)(3,3,5-trimethyl-2,3-dihydro-1H-pyrrolo[3,2-b]pyridin-1-yl)methanone;
141 (3,3-dimethyl-2,3-dihydro-1H-pyrrolo[3,2-b]pyridin-1-yl)(8-(2-fluorobenzyl)-2,8-diazaspiro[4.5]decan-2-yl)methanone;
142 4-((2-(3,3-dimethyl-2,3-dihydro-1H-pyrrolo[3,2-b]pyridine-1-carbonyl)-2,8-diazaspiro[4.5]decan-8-yl)methyl)-2-fluorobenzonitrile;
143 5-((2-(3,3-dimethyl-2,3-dihydro-1H-pyrrolo[3,2-b]pyridine-1-carbonyl)-2,8-diazaspiro[4.5]decan-8-yl)methyl)-2-fluorobenzonitrile;
144 (3,3-dimethyl-2,3-dihydro-1H-pyrrolo[3,2-b]pyridin-1-yl)(8-((tetrahydro-2H-pyran-4-yl)methyl)-2,8-diazaspiro[4.5]decan-2-yl)methanone;
145 (3,3-dimethyl-2,3-dihydro-1H-pyrrolo[3,2-b]pyridin-1-yl)(9-(2-fluorobenzyl)-3,9-diazaspiro[5.5]undecan-3-yl)methanone;
146 4-((9-(3,3-dimethyl-2,3-dihydro-1H-pyrrolo[3,2-b]pyridine-1-carbonyl)-3,9-diazaspiro[5.5]undecan-3-yl)methyl)-2-fluorobenzonitrile;
147 5-((9-(3,3-dimethyl-2,3-dihydro-1H-pyrrolo[3,2-b]pyridine-1-carbonyl)-3,9-diazaspiro[5.5]undecan-3-yl)methyl)-2-fluorobenzonitrile;
148 (8-(2,5-difluorobenzyl)-2,8-diazaspiro[4.5]decan-2-yl)(3,3-dimethyl-2,3-dihydro-1H-pyrrolo[3,2-b]pyridin-1-yl)methanone;
149 (3,3-dimethyl-2,3-dihydro-1H-pyrrolo[3,2-b]pyridin-1-yl)(8-(4-fluorobenzyl)-2,8-diazaspiro[4.5]decan-2-yl)methanone;
150 (8-(2,6-difluorobenzyl)-2,8-diazaspiro[4.5]decan-2-yl)(3,3-dimethyl-2,3-dihydro-1H-pyrrolo[3,2-b]pyridin-1-yl)methanone;
151 4-((2-(3,3-dimethyl-2,3-dihydro-1H-pyrrolo[3,2-b]pyridine-1-carbonyl)-2,8-diazaspiro[4.5]decan-8-yl)methyl)benzonitrile;
152 (3,3-dimethyl-2,3-dihydro-1H-pyrrolo[3,2-b]pyridin-1-yl)(8-(3-fluorobenzyl)-2,8-diazaspiro[4.5]decan-2-yl)methanone;
153 3,3-dimethyl-2,3-dihydro-1H-pyrrolo[3,2-b]pyridin-1-yl)(8-((3-fluoropyridin-2-yl)methyl)-2,8-diazaspiro[4.5]decan-2-yl)methanone;
154 (3,3-dimethyl-2,3-dihydro-1H-pyrrolo[3,2-b]pyridin-1-yl)(8-((5-fluoropyridin-2-yl)methyl)-2,8-diazaspiro[4.5]decan-2-yl)methanone;
155 (3,3-dimethyl-2,3-dihydro-1H-pyrrolo[3,2-b]pyridin-1-yl)(8-((6-(trifluoromethyl)pyridin-3-yl)methyl)-2,8-diazaspiro[4.5]decan-2-yl)methanone;
156 (3,3-dimethyl-2,3-dihydro-1H-pyrrolo[3,2-b]pyridin-1-yl)(8-(2-(tetrahydro-2H-pyran-4-yl)ethyl)-2,8-diazaspiro[4.5]decan-2-yl)methanone;
157 4-((2-(3,3-dimethyl-2,3-dihydro-1H-pyrrolo[3,2-b]pyridine-1-carbonyl)-2,8-diazaspiro[4.5]decan-8-yl)methyl)-3-fluorobenzonitrile;
158 5-((2-(3,3-dimethyl-2,3-dihydro-1H-pyrrolo[3,2-b]pyridine-1-carbonyl)-2,8-diazaspiro[4.5]decan-8-yl)methyl)-2,4-difluorobenzonitrile;
159 (7-benzyl-2,7-diazaspiro[3.5]nonan-2-yl)(3,3-dimethyl-2,3-dihydro-1H-pyrrolo[3,2-b]pyridin-1-yl)methanone;
160 (9-(2-fluorobenzyl)-3,9-diazaspiro[5.5]undecan-3-yl)(3,3,5-trimethyl-2,3-dihydro-1H-pyrrolo[3,2-b]pyridin-1-yl)methanone;
161 (9-((tetrahydro-2H-pyran-4-yl)methyl)-3,9-diazaspiro[5.5]undecan-3-yl)(3,3,5-trimethyl-2,3-dihydro-1H-pyrrolo[3,2-b]pyridin-1-yl)methanone;
162 2-fluoro-5-((9-(3,3,5-trimethyl-2,3-dihydro-1H-pyrrolo[3,2-b]pyridine-1-carbonyl)-3,9-diazaspiro[5.5]undecan-3-yl)methyl)benzonitrile;
163 2-fluoro-4-((9-(3,3,5-trimethyl-2,3-dihydro-1H-pyrrolo[3,2-b]pyridine-1-carbonyl)-3,9-diazaspiro[5.5]undecan-3-yl)methyl)benzonitrile;
164 (9-(2,5-difluorobenzyl)-3,9-diazaspiro[5.5]undecan-3-yl)(3,3-dimethyl-2,3-dihydro-1H-pyrrolo[3,2-b]pyridin-1-yl)methanone;
165 4-((9-(3,3-dimethyl-2,3-dihydro-1H-pyrrolo[3,2-b]pyridine-1-carbonyl)-3,9-diazaspiro[5.5]undecan-3-yl)methyl)-3-fluorobenzonitrile;
166 (3,3-dimethyl-2,3-dihydro-1H-pyrrolo[3,2-b]pyridin-1-yl)(9-((tetrahydro-2H-pyran-4-yl)methyl)-3,9-diazaspiro[5.5]undecan-3-yl)methanone;
167 5-((9-(3,3-dimethyl-2,3-dihydro-1H-pyrrolo[3,2-b]pyridine-1-carbonyl)-3,9-diazaspiro[5.5]undecan-3-yl)methyl)-2,4-difluorobenzonitrile;
168 ((1r,4r)-4-((3,5-difluorobenzyl)(methyl)amino)cyclohexyl)(3,3,5-trimethyl-2,3-dihydro-1H-pyrrolo[3,2-b]pyridin-1-yl)methanone;
169 ((1r,4r)-4-((3-fluorobenzyl)(methyl)amino)cyclohexyl)(3,3,5-trimethyl-2,3-dihydro-1H-pyrrolo[3,2-b]pyridin-1-yl)methanone;
170 ((1r,4r)-4-((3,4-difluorobenzyl)(methyl)amino)cyclohexyl)(3,3,5-trimethyl-2,3-dihydro-1H-pyrrolo[3,2-b]pyridin-1-yl)methanone;
171 ((1r,4r)-4-((2,6-difluorobenzyl)(methyl)amino)cyclohexyl)(3,3,5-trimethyl-2,3-dihydro-1H-pyrrolo[3,2-b]pyridin-1-yl)methanone;
172 ((1r,4r)-4-((2,4-difluorobenzyl)(methyl)amino)cyclohexyl)(3,3,5-trimethyl-2,3-dihydro-1H-pyrrolo[3,2-b]pyridin-1-yl)methanone;
173 ((1r,4r)-4-((2,5-difluorobenzyl)(methyl)amino)cyclohexyl)(3,3,5-trimethyl-2,3-dihydro-1H-pyrrolo[3,2-b]pyridin-1-yl)methanone;
174 ((1r,4r)-4-((2,3-difluorobenzyl)(methyl)amino)cyclohexyl)(3,3,5-trimethyl-2,3-dihydro-1H-pyrrolo[3,2-b]pyridin-1-yl)methanone;
175 (3,3-dimethyl-2,3-dihydro-1H-pyrrolo[3,2-b]pyridin-1-yl)((1r,4r)-4-((2-fluorobenzyl)(methyl)amino)cyclohexyl)methanone;
176 ((1r,4r)-4-((2,5-difluorobenzyl)(methyl)amino)cyclohexyl)(3,3-dimethyl-2,3-dihydro-1H-pyrrolo[3,2-b]pyridin-1-yl)methanone;
177 ((1r,4r)-4-(methyl((2-(trifluoromethyl)pyridin-4-yl)methyl)amino)cyclohexyl)(3,3,5-trimethyl-2,3-dihydro-1H-pyrrolo[3,2-b]pyridin-1-yl)methanone;
178 ((1r,4r)-4-(((3-fluoropyridin-2-yl)methyl)(methyl)amino)cyclohexyl)(3,3,5-trimethyl-2,3-dihydro-1H-pyrrolo[3,2-b]pyridin-1-yl)methanone;
179 ((1r,4r)-4-(((5-fluoropyridin-2-yl)methyl)(methyl)amino)cyclohexyl)(3,3,5-trimethyl-2,3-dihydro-1H-pyrrolo[3,2-b]pyridin-1-yl)methanone;
180 2-fluoro-4-((methyl((1r,4r)-4-(3,3,5-trimethyl-2,3-dihydro-1H-pyrrolo[3,2-b]pyridine-1-carbonyl)cyclohexyl)amino)methyl)benzonitrile;
181 2-fluoro-5-((methyl((1r,4r)-4-(3,3,5-trimethyl-2,3-dihydro-1H-pyrrolo[3,2-b]pyridine-1-carbonyl)cyclohexyl)amino)methyl)benzonitrile;
182 ((1r,4r)-4-((4-fluorobenzyl)(methyl)amino)cyclohexyl)(3,3,5-trimethyl-2,3-dihydro-1H-pyrrolo[3,2-b]pyridin-1-yl)methanone;
183 ((1r,4r)-4-((2-fluorobenzyl)(methyl)amino)cyclohexyl)(3,3,5-trimethyl-2,3-dihydro-1H-pyrrolo[3,2-b]pyridin-1-yl)methanone;
184 ((1r,4r)-4-(methyl((6-(trifluoromethyl)pyridin-3-yl)methyl)amino)cyclohexyl)(3,3,5-trimethyl-2,3-dihydro-1H-pyrrolo[3,2-b]pyridin-1-yl)methanone;
185 ((1r,4r)-4-(methyl((tetrahydro-2H-pyran-4-yl)methyl)amino)cyclohexyl)(3,3,5-trimethyl-2,3-dihydro-1H-pyrrolo[3,2-b]pyridin-1-yl)methanone;
186 ((1r,4r)-4-(benzyl(methyl)amino)cyclohexyl)(3,3-dimethyl-5-(trifluoromethyl)-2,3-dihydro-1H-pyrrolo[3,2-b]pyridin-1-yl)methanone;
187 ((1r,4r)-4-(benzyl(methyl)amino)cyclohexyl)(6-fluoro-3,3-dimethyl-2,3-dihydro-1H-pyrrolo[3,2-b]pyridin-1-yl)methanone and
188 ((1r,4r)-4-((2-fluorobenzyl)amino)cyclohexyl)(3,3,5-trimethyl-2,3-dihydro-1H-pyrrolo[3,2-b]pyridin-1-yl)methanone.
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compounds which are selected act as ligands with great affinity for sigma receptors, especially sigma-1 (σ₁) and/or sigma-2 (σ₂) receptors, and especially compounds which have a binding expressed as Kᵢ (affinity value) responding to the following scales:
Kᵢ(σ₁) is preferably <1000 nM, more preferably <500 nM, even more preferably <100 nM; and
Kᵢ(σ₂) is preferably <1000 nM, more preferably <500 nM, even more preferably <100 nM.

In a particular embodiment, the compounds selected showing a binding expressed as Kᵢ which is Kᵢ(σ₁)>=1000 nM, show a binding, expressed as percentage of inhibition, of between 1% and 50%. In another particular embodiment, the compounds selected showing a binding expressed as Kᵢ which is Kᵢ(σ₂)>=1000 nM, show a binding, expressed as percentage of inhibition, of between 1% and 50%.

The binding of the compounds, expressed as Ki or as percentage of inhibition, is measured as explained in the Examples below.

In another aspect, the invention refers to a process for the preparation of a compound of formula (I) as defined above.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. The compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

The compounds of formula (I) can be obtained by following the methods described below. As it will be obvious to one skilled in the art, the exact method used to prepare a given compound may vary depending on its chemical structure.

Two different general methods have been developed for obtaining the compounds of the invention, depending on the nature of the atom wherein group A is attached to the carbonyl group present in the compound of formula (I), as described below in methods A and B, and further detailed in Schemes 1 to 2.

### Method A

A one-step process is described for the preparation of compounds of general formula (I) wherein group A is attached through a N atom, starting from a compound of formula (II) and a cyclic or acyclic amine of formula (III), as shown in the following scheme:

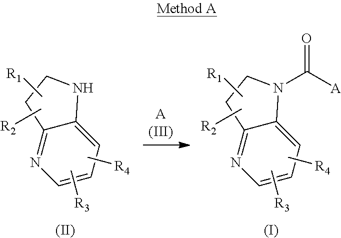

wherein R₁, R₂, R₃, R₄ and A have the meanings as defined before.

Thus, in another aspect, the invention refers to a process for the preparation of a compound of formula (I) wherein group A is attached through a N atom, said process comprising reacting a compound of formula (II)

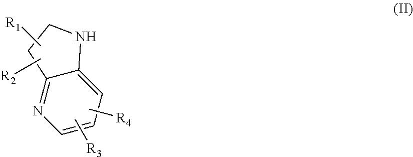

with a cyclic or acyclic amine A, wherein R₁, R₂, R₃, R₄ and A have the same meanings as defined before for a compound of formula (I).

The preparation of a urea compound of formula (I) from a N-containing cyclic reagent of formula (II) and an amino compound of formula (III) can be carried out under conventional urea formation conditions described in the literature (see J. Med. Chem. 2020, 63, 6, 2751-2788) using a carbonyl source such as triphosgene, phosgene, 1,1′-carbonyldiimidazole (CDI) or 1,1′-carbonylbisbenzotriazole (CBT), preferably triphosgene; optionally in the presence of an organic base such as N,N-diisopropylethylamine or triethylamine, or in the case of CDI optionally in the presence of trimethylaluminum; in a suitable solvent such as N,N-dimethylformamide or dichloromethane or mixtures thereof, or other aprotic solvents, and at a suitable temperature, preferably at room temperature.

In a preferred embodiment, the invention refers to the process for the preparation of a compound of formula (I) wherein group A is attached through a N atom said process comprising treating a compound of formula (II)

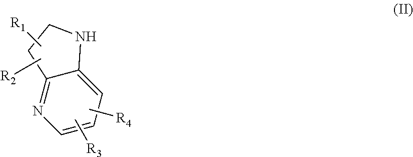

with a cyclic or acyclic amine A using a carbonyl source, such as triphosgene, phosgene, 1,1′-carbonyldiimidazole or 1,1′-carbonylbisbenzotriazole, in a suitable solvent, such as N,N-dimethylformamide or dichloromethane or mixtures thereof, or other aprotic solvents, and at a suitable temperature, preferably at room temperature.

Alternatively, the reaction can be conducted in two steps by treating either (II) or (III) with a suitable chloroformate such as 4-nitrophenyl chloroformate, in a suitable solvent such as dichloromethane, in the presence of a base such as N,N-diisopropylethylamine or triethylamine, to render a urethane intermediate and finally reacting with the other component, either (III) or (II), to render a compound of formula (I). The aminolysis reaction of the urethane intermediate is carried out in a suitable solvent such as N,N-dimethylformamide, at a suitable temperature, preferably heating.

### Method B

A one-step process is described for the preparation of amide compounds of formula (I) wherein group A is attached through a C atom, starting from a compound of formula (II) and a cyclic or acyclic carboxylic acid of formula (IV), as shown in the following scheme:

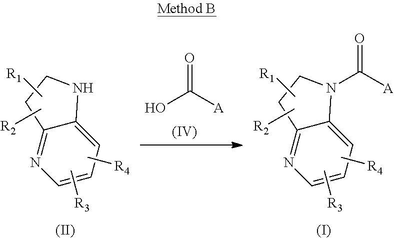

wherein R₁, R₂, R₃, R₄ and A have the meanings as defined before.

Thus, in another aspect, the invention refers to a process for the preparation of a compound of formula (I) wherein group A is attached through a C atom, said process comprising reacting a compound of formula (II)

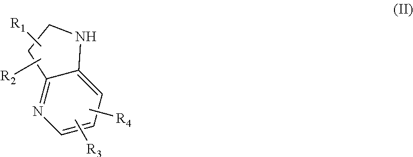

with a cyclic or acyclic carboxylic acid of formula (IV)

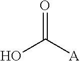

wherein R₁, R₂, R₃, R₄ and A have the same meanings as defined before for a compound of formula (I).

The preparation of an amide compound of formula (I) from a N-containing cyclic reagent of formula (II) and an acid compound of formula (IV) can be carried out under conventional amidation conditions, preferably using a suitable coupling reagent such as N-[(dimethylamino)-1H-1,2,3-triazolo-[4,5-b]pyridin-1-ylmethylene]-N-methylmethanaminium hexafluorophosphate N-oxide (HATU), N-(3-dimethylaminopropyl)-N′-ethylcarbodiimide (EDC), N,N,N′,N′-tetramethyl-O-(1H-benzotriazol-1-yl)uronium hexafluorophosphate (HBTU), (benzotriazol-1-yloxy)tripyrrol idinophosphonium hexafluorophosphate (PyBOP), dicyclohexylcarbodiimide (DCC), or propylphosphonic anhydride (T3P), optionally in the presence of 1-hydroxybenzotriazole, optionally in the presence of an organic base such as N,N-diisopropylethylamine, N-methylmorpholine or triethylamine, optionally in the presence of an activating agent such as 4-dimethylaminopyridine, in a suitable solvent such as N,N-dimethylformamide or dichloromethane, and at a suitable temperature, preferably at room temperature. Alternatively, the amidation can be performed in two steps by first converting an acid of formula (IV) into its corresponding acyl halide or mixed anhydride following standard conditions described in the literature, and then reacting it with a compound of formula (II) in a suitable solvent, such as dichloromethane, tetrahydrofuran, ethyl acetate or ethyl acetate-water mixtures; in the presence of an organic base such as triethylamine or N,N-diisopropylethylamine or an inorganic base such as K₂CO₃; and at a suitable temperature, preferably comprised between 0° C. and the reflux temperature. Additionally, an activating agent such as 4-dimethylaminopyridine can be also used.

The compounds of formula (II), (III) and (IV) are commercially available or can be synthesized following common procedures described in the literature. In this regard, the synthesis of compounds of formula (II) has been described in WO2019020792.

In an alternative way to Methods A and B, the compounds of formula (I) wherein A is one of the following groups:

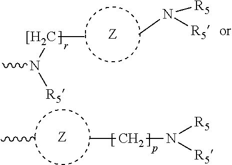

i.e. (I-1) and (I-2) respectively (see below), can be prepared by introducing the substituent NR₅R_{5′} starting from a keto precursor (either an aldehyde or a ketone) of formula (V-1) or (V-2) respectively and an amine of formula (VI) under reductive amination conditions, as shown in Scheme 1:

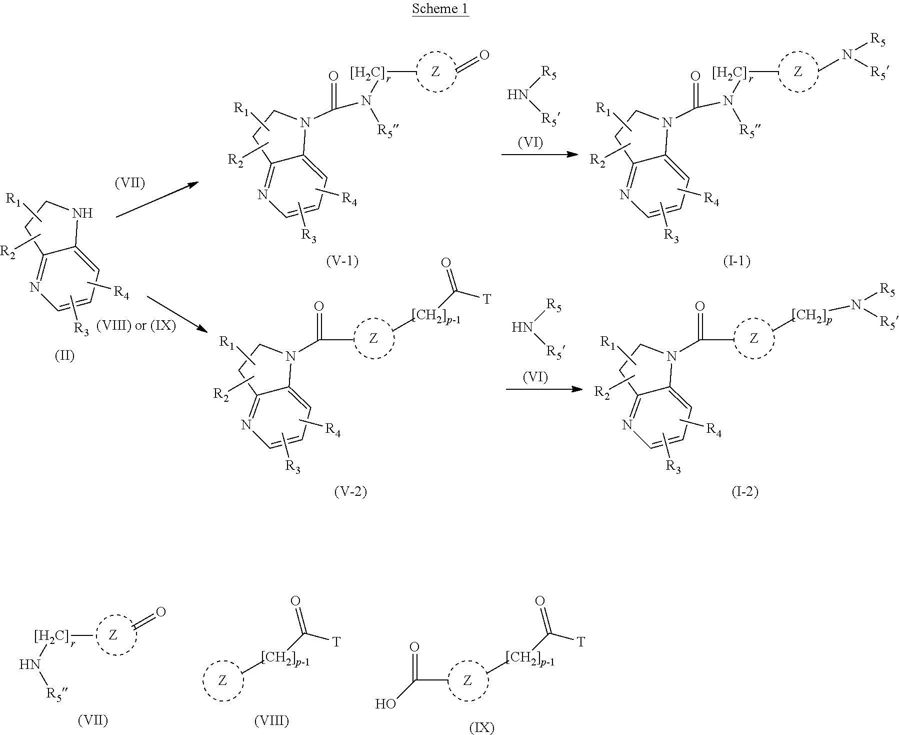

wherein R₁, R₂, R₃, R₄, R₅, R₅′, R₅″, p, r and Z have the meanings as defined above and T represents H or alkyl.

The reductive amination reaction is carried out in the presence of a reductive reagent, such as sodium triacetoxyborohydride, sodium cyanoborohydride or sodium borohydride, in a suitable solvent, preferably 1,2-dichloroethane, dichloromethane, tetrahydrofuran, methanol or ethanol, optionally in the presence of an acid (such as acetic acid) or a base (such as N,N-diisopropylethylamine), optionally pre-forming the corresponding imine before the addition of the reductive reagent, and preferably the reaction is carried out at room temperature.

The keto compounds of formula (V-1) and (V-2) can be prepared by reaction of a compound of formula (II) with a suitable amino partner of formula (VII) or (VIII) under the urea formation conditions already described in Method A or in the case of (V-2), alternatively, by reaction with an acid of formula (IX) under the amidation conditions described in Method B.

In another alternative way to Methods A and B, starting from a precursor compound wherein R₅ is absent (R₅ is hydrogen), i.e. a compound of formula (X-1), (X-2), (X-3) or (X-4), it can be transformed into a compound wherein R₅ is present, that is a compound of formula (I-1), (I-2), (I-3) or (I-4), as shown in Scheme 2:

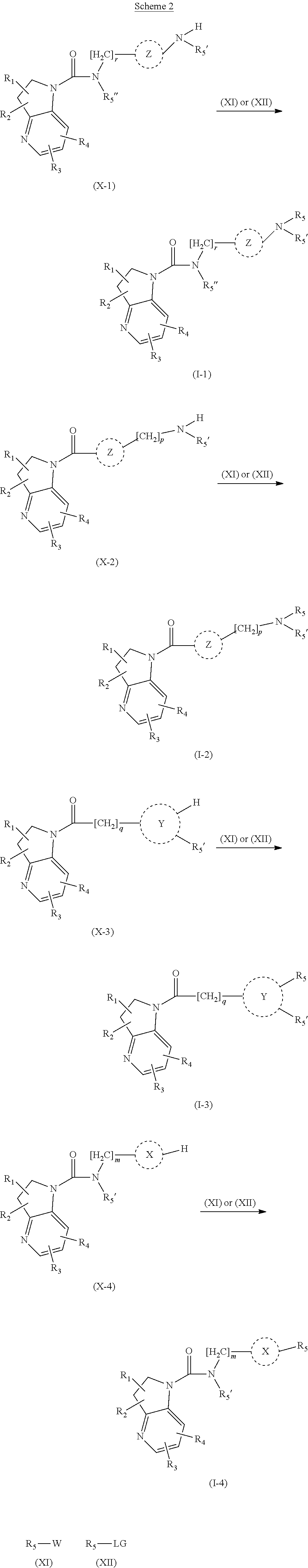

wherein R₁, R₂, R₃, R₄, R₅, R₅′, R₅″, M, p, q, r, X, Y and Z have the meanings as defined above, W represents a keto group (either an aldehyde or a ketone) and LG represents a suitable leaving group (such as chloro, bromo, iodo, mesylate, tosylate, nosylate or triflate).

The reaction can be conducted by treating a compound of formula (X-1), (X-2), (X-3) or (X-4) with a keto compound of formula (XI) under standard reductive amination conditions such as those described in Scheme 1 for the reaction of a compound of formula (V-1) or (V-2) with an amine of formula (VI). Alternatively, the reaction can be carried out under standard alkylation conditions by reacting a compound of formula (X-1), (X-2), (X-3) or (X-4) with an alkylating agent of formula (XII), in a suitable solvent, such as acetonitrile, N,N-dimethylformamide, dimethylsulfoxide, dichloromethane, tetrahydrofuran or 1,4-dioxane; in the presence of an inorganic base such as K₂CO₃, Cs₂CO₃ or a strong base such as sodium hydride or potassium tert-butoxide, or an organic base such as triethylamine or N,N-diisopropylethylamine, at a suitable temperature comprised between room temperature and the reflux temperature.

Thus, in another embodiment, the invention refers to the use of a compound selected from:

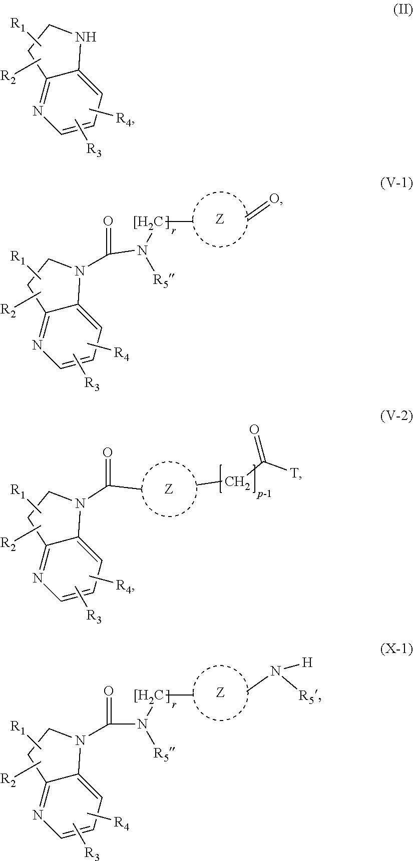

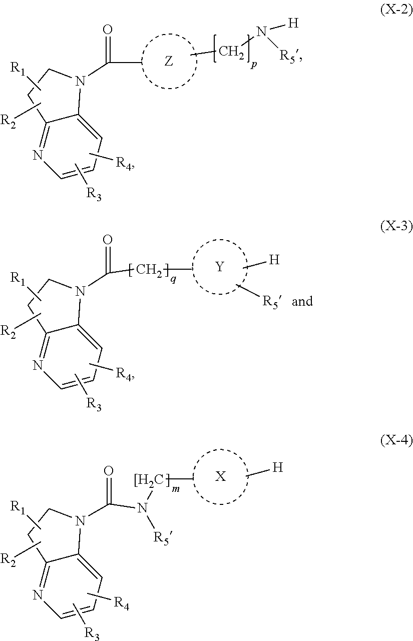

wherein R₁, R₂, R₃, R₄, R₅′, R₅″, X, Y, Z, m, p, q, and r have the same meaning as indicated before for a compound of formula (I) and T represents hydrogen or alkyl, for the manufacture of a compound of formula (I).

The precursor compounds of formula (X-1), (X-2), (X-3) or (X-4) can be prepared following the procedures described above in Methods A and B and Scheme 1 for the preparation of a compound of formula (I), starting from a compound of formula (II) and using the corresponding reagents (III), (IV) or (VI) wherein R₅ is hydrogen.

The compounds of formula (VI), (VII), (VIII), (IX), (XI) and (XII) are commercially available or can be synthesized following common procedures described in the literature.

Moreover, certain compounds of the present invention can also be obtained starting from other compounds of formula (I) by appropriate conversion reactions of functional groups, in one or several steps, using well-known reactions in organic chemistry under standard experimental conditions. For example, starting from a compound of formula (I) wherein R_{5′}, R_{5″} or R_{5′″} is hydrogen, R_{5′}, R_{5″} or R_{5′″} can be transformed into an alkyl group under the reductive amination reaction conditions described above.

In some of the processes described above, it may be necessary to protect the amino groups present in any of the compounds with suitable protecting groups, such as for example Boc (tert-butoxycarbonyl), Fmoc (fluorenylmethyloxycarbonyl), Cbz (benzyloxycarbonyl) or benzyl. The procedures for the introduction and removal of these protecting groups are well known in the art and can be found thoroughly described in the literature. As a way of example, for Boc as protecting group, the deprotection can be conducted by adding a solution of a strong acid such as HCl, in a suitable solvent such as diethyl ether, 1,4-dioxane or methanol, or with trifluoroacetic acid in dichloromethane. For Fmoc as protecting group, the deprotection is usually performed under basic media, such as for example diethylamine or piperidine in dichloromethane or N,N-dimethylformamide. When the protecting group is Cbz or benzyl, the deprotection reaction is preferably carried out by hydrogenation under hydrogen atmosphere and metal catalysis, preferably by the use of palladium or palladium hydroxide over charcoal as catalyst, in a suitable solvent such as methanol or ethanol, optionally in the presence of an acid such as acetic acid or hydrochloric acid.

Finally, a compound of formula (I) can be obtained in enantiopure form by resolution of a racemic compound of formula (I) or a diastereomeric mixture, either by chiral preparative HPLC or by crystallization of a diastereomeric salt or co-crystal. Alternatively, the resolution step can be carried out at a previous stage, using any suitable intermediate.

Another aspect of the invention refers to a pharmaceutical composition which comprises a compound according to the invention as described above according to formula (I) or a pharmaceutically acceptable salt thereof, prodrug, solvate or stereoisomer thereof, and a pharmaceutically acceptable carrier, adjuvant or vehicle. The present invention thus provides pharmaceutical compositions comprising a compound of this invention, or a pharmaceutically acceptable salt, prodrug, solvate or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrups or solutions and may contain conventional excipients known in the art such as binding agents, for example syrup, acacia, gelatine, sorbitol, tragacanth, or polyvinylpyrrolidone; fillers, for example lactose, sugar, maize starch, calcium phosphate, sorbitol or glycine; tableting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tableting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to a compound of formula (I) as described above, or a pharmaceutical acceptable salt or isomer thereof for use in therapy.

Another aspect of the invention refers to a compound of formula (I), or a pharmaceutically acceptable salt or isomer thereof, for use in the treatment or prophylaxis of pain.

Preferably, the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain. In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment or prevention a therapeutically effective amount of a compound as above defined or a pharmaceutical composition thereof. Among the pain syndromes that can be treated or prevented are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

### EXAMPLES

In the next examples the preparation of both intermediate compounds as well as compounds according to the invention are disclosed.

The following abbreviations are used in the examples:
ACN: acetonitrile
Aq: aqueous
CH: cyclohexane
DCM: dichloromethane
DCE: dichloroethane
DIPEA: N,N-diisopropylethylamine
DME: 1,2-dimethoxyethane
DMF: N,N-dimethylformamide
DMSO: dimethylsulfoxide
EDC: 3-(((ethylimino)methylene)amino)-N,N-dimethylpropan-1-amine
EtOAc: ethyl acetate
EtOH: ethanol
EX: example
h: hour/s
HATU: O-(7-azabenzotriazol-1-yl)-N,N,N′,N′-tetramethyluronium hexafluorophosphate
HOBt: 1H-benzo[d][1,2,3]triazol-1-ol
HPLC: high performance liquid chromatography
IPC: in process control
MeOH: methanol
MS: mass spectrometry
min.: minutes
NaBH(OAc)₃: sodium triacetoxy borohydride
Quant: quantitative
Ret.: retention
r.t.: room temperature
Sat: saturated
Sol.: solution
SPhos: 2-Dicyclohexylphosphino-2′,6′-di methoxybiphenyl
TEA: triethylamine
TFA: trifluoroacetic acid
THF: tetrahydrofuran
wt: weight

The following methods were used to determine the HPLC-MS spectra:

### Method A:

Column: Kinetex EVO 50×4.6 mm, 2.6 um
Temperature: 40° C.
Flow: 1.5 mL/min
Gradient: NH₄HCO₃ pH 8: ACN (95:5)-0.5 min-(95:5)-6.5 min-(0:100)-2 min-(0:100)
Sample dissolved approx. 1 mg/mL in NH₄HCO₃ pH 8/ACN

### Method B:

Column ZORBAX Extend-C18 RRHD 2.1×50 mm, 1.8 μm
Temperature 35° C.
Flow rate 0.61 mL/min; A: NH 4 HCO₃ 10 mM, B: MeCN
Gradient: 0.3 min 98% A, 98% A to 100% B in 2.65 min; isocratic 2.05 min 100% B.

### Method C:

Column ZORBAX Extend-C18 RRHD 2.1×50 mm, 1.8 μm
Temperature 35° C.
Flow rate 0.61 mL/min; A: NH₄HCO₃ 10 mM, B: MeCN, C: MeOH+0.1% formic acid
Gradient: 0.3 min 98% A, 98% A to 0:95:5 A:B:C in 2.7 min; 0:95:5 A:B:C to 100% B in 0.1 min; isocratic 2 min 100% B.

### Synthesis of Intermediates

### Intermediate 1A: (R)-1-(3-Methoxyphenyl)-N′,N′-dimethylethane-1,2-diamine

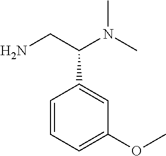

Step 1. (R)-2-(Dimethylamino)-2-(3-methoxyphenyl)acetic acid: To a solution of (R)-2-amino-2-(3-methoxyphenyl)acetic acid (0.5 g, 2.76 mmol) and formaldehyde (2.45 mL, 24.8 mmol) in 2,2,2-trifluoroethanol (12.5 mL), NaBH₄ (447 mg, 11.8 mmol) was added portionwise. The mixture was heated at 80° C. for 7 h. The suspension formed during the reaction was filtered through a sintered funnel, washing with 2,2,2-trifluoroethanol. The filtrate was evaporated to dryness and the residue was purified by flash chromatography, silica gel, gradient DCM to MeOH:DCM (1:4), to give the title compound (380 mg, 66% yield).

Step 2. (R)-2-(Dimethylamino)-2-(3-methoxyphenyl)acetamide: To a solution of the product obtained in Step 1 (380 mg, 1.82 mmol) in DMF (14 mL), HOBt hydrate (491 mg, 3.21 mmol) and EDC hydrochloride (666 mg, 3.47 mmol) were added and the mixture was stirred at r.t. for 30 min. Aqueous ammonia (32 wt % solution, 0.89 mL, 7.26 mmol) was added and the reaction mixture was stirred at r.t. overnight. Water was added and the aqueous phase was extracted with EtOAc and finally with DCM. The combined organic extracts were washed with 5% NaHCO₃ aq. sol., dried over MgSO₄, filtered and concentrated under vacuum to give the title compound (336 mg, 72% yield).

Step 3. Title compound: To a solution of the product obtained in Step 2 (272 mg, 1.31 mmol) in THF (6 mL), cooled at 0° C., borane-methyl sulfide complex (0.5 mL, 5.22 mmol) was added dropwise. The reaction mixture was heated at 65° C. overnight. Then, MeOH was added carefully and the resulting mixture was stirred at r.t. for 30 min. The solvent was evaporated to dryness and the residue was partitioned between cold water and DCM. The phases were separated and the aqueous phase was extracted with DCM. The combined organic extracts were washed with water and brine, dried over MgSO₄, filtered and concentrated under vacuum. HPLC-MS analysis of the crude showed incomplete reaction, thus the evaporation residue was submitted to a second reaction cycle. It was dissolved again in THF (6 mL), cooled at 0° C. and borane-methyl sulfide complex (0.5 mL, 5.22 mmol) was added dropwise. The resulting mixture was heated at 65° C. overnight. After cooling down to r.t., MeOH was carefully added and the reaction mixture was stirred for 30 min. The solvent was evaporated to dryness and the residue thus obtained was directly purified by flash chromatography, silica gel, gradient DCM to MeOH:DCM (1:4), to give the title compound (74 mg, 29% yield).

This method was used for the preparation of Intermediates 1B-1C using suitable starting materials:

| INT | Structure | Chemical name |
|---|---|---|
| 1B | 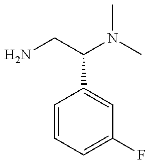 | (R)-1-(3-fluorophenyl)- N¹N¹-dimethylethane-1,2- diamine |
| 1C | 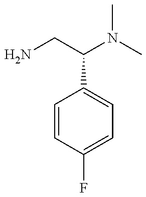 | (R)-1-(4-fluorophenyl)- N¹,N¹-dimethylethane-1,2- diamine |

### Intermediate 1D: (R)-tert-Butyl (2-amino-1-phenylethyl)(ethyl)carbamate

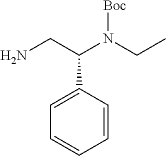 [SMILES: CCN(C(=O)OC(C)(C)C)[C@@H](CN)c1ccccc1]

Step 1. (R)-2-((tert-Butoxycarbonyl)(ethyl)amino)-2-phenylacetic acid: To a solution of (R)-2-((tert-butoxycarbonyl)amino)-2-phenylacetic acid (2.0 g, 7.96 mmol) and iodoethane (6.4 mL, 80 mmol) in dry THF (35 mL), cooled at 0° C., NaH (60 wt % dispersion in mineral oil, 3.18 g, 80 mmol) was added portionwise. The mixture was stirred at r.t. overnight. IPC analysis by HPLC-MS indicated incomplete reaction. The reaction mixture was cooled to 0° C., iodoethane (6.4 mL, 80 mmol) and NaH (60 wt % dispersion in mineral oil, 3.18 g, 80 mmol) were added sequentially, and the resulting mixture was again stirred at r.t. overnight. Water was added to quench the reaction and THF was evaporated. The resulting basic aqueous phase was washed with EtOAc (that was discarded) and acidified with citric acid (5 wt % solution) to pH 3. The acidic aqueous phase was extracted with EtOAc and the combined organic extracts were dried over MgSO₄, filtered and concentrated under vacuum. HPLC-MS analysis of the crude showed incomplete reaction, thus it was submitted to a second reaction cycle. The crude was dissolved in THF (35 mL), iodoethane (5 mL, 64 mmol) was added, and the mixture was cooled at 0° C. NaH (60 wt % dispersion in mineral oil, 2.5 g, 64 mmol) was added portionwise and the mixture was stirred at r.t. overnight and finally it was heated at 50° C. for 2 days. Water was added, THF was evaporated and the resulting basic aqueous phase was washed with EtOAc and acidified with citric acid (5 wt % solution) to pH 3. The acidic aqueous phase was extracted with EtOAc and the combined organic extracts were dried over MgSO₄, filtered and concentrated under vacuum to give the title compound (1.1 g, 50% yield).

Step 2. (R)-tert-Butyl (2-amino-2-oxo-1-phenylethyl)(ethyl)carbamate: Starting from the product obtained in Step 1 (1.1 g, 3.95 mmol) and following the experimental procedure described in Step 2 of Intermediate 1A, the title compound was obtained (508 mg, 46% yield).

Step 3. Title compound: To a solution of the product obtained in Step 2 (508 mg, 1.82 mmol) in THF (20 mL), cooled at 0° C., borane solution (1 M in THF, 11 mL, 11 mmol) was added dropwise and the reaction mixture was heated at 65° C. overnight. After cooling down to r.t., MeOH was carefully added and the reaction mixture was stirred until gas evolution ceased. Then, the solvent was evaporated to dryness. The residue was purified by flash chromatography, silica gel, gradient DCM to MeOH:DCM (1:4), to give the title compound (184 mg, 38% yield).

This method was used for the preparation of Intermediates 1E-1F using suitable starting

| INT | Structure | Chemical name |
|---|---|---|
| 1E | 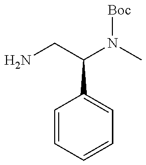 [SMILES: CN(C(=O)OC(C)(C)C)[C@H](CN)c1ccccc1] | (S)-tert-butyl (2-amino-1- phenylethyl)(methyl)carba- mate |
| 1F | 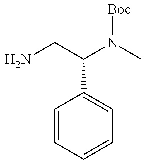 [SMILES: CN(C(=O)OC(C)(C)C)[C@@H](CN)c1ccccc1] | (R)-tert-butyl (2-amino-1- phenylethyl)(methyl)carba- mate |

### Intermediate 1G: (R)—N¹,N¹,N²-tTrimethyl-1-phenylethane-1,2-diamine

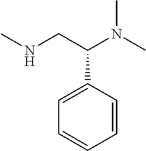

Step 1. (R)-2-Amino-N-methyl-2-phenylacetamide: To (R)-methyl 2-amino-2-phenylacetate hydrochloride (2.0 g, 9.92 mmol), cooled at 10-15° C., methylamine solution (40 wt % in water, 3.43 mL, 39.7 mmol) was slowly added and the reaction mixture was stirred at r.t. for 1 h. Brine was added and it was extracted with a mixture of THF:EtOAc (1:1). The combined organic extracts were dried over MgSO₄, filtered and concentrated under vacuum to give the title compound (1.39 g, 86% yield).

Step 2. (R)-2-(Dimethylamino)-N-methyl-2-phenylacetamide: To a solution of the product obtained in Step 1 (1.39 g, 8.5 mmol) and formaldehyde (8.2 mL, 110 mmol) in MeOH (65 mL), previously purged with nitrogen, palladium (10 wt % on charcoal, wet, 452 mg) was added. The resulting suspension was heated at 65° C. for 90 min, then the temperature was lowered to 45° C. and the reaction flask was purged with H₂ by bubbling it through the suspension. The reaction was stirred at this temperature for 2.5 h. After cooling down to r.t., the catalyst was filtered off over a pad of Celite and the filtrate was evaporated to dryness. The residue was partitioned between water and DCM. The phases were separated and the aqueous phase was extracted with DCM. The combined organic phases were dried over MgSO₄, filtered and concentrated to dryness. HPLC-MS analysis of the crude showed 80% conversion, thus it was submitted to a second reaction cycle. The residue was re-dissolved in MeOH (65 mL) and formaldehyde (4.1 mL, 55 mmol) and palladium (10 wt % on charcoal, wet, 250 mg) was added. The suspension was heated at 65° C. under N₂ atmosphere for 90 min, then, after cooling to 45° C., H₂ was bubbled through the suspension and the reaction mixture was further stirred for 2.5 h. The catalyst was filtered off and the solvent was evaporated. The residue was partitioned between water and DCM, the phases were separated and the aqueous phase was extracted with DCM. The combined organic extracts were dried over MgSO₄, filtered and concentrated to dryness to give the title compound (1.5 g, 92% yield).

Step 3. Title compound: To a solution of the product obtained in Step 2 (1.38 g, 7.18 mmol) in THF (144 mL), LiAlH₄ solution (1 M in THF, 36 mL, 36 mmol) was added dropwise under a nitrogen atmosphere. The reaction mixture was heated to reflux overnight. Then, additional LiAlH₄ dolution (1 M in THF, 36 mL, 36 mmol) was added dropwise and the reaction mixture was again heated to reflux overnight. Then, it was cooled to r.t. Water (1.7 mL), 1 N aq. NaOH (1.7 mL) and water (4.2 mL) were added sequentially and the mixture was stirred at r.t. for 1 h. The resulting suspension was filtered through a pad of Celite, washing the cake with EtOAc. The filtrate was dried over MgSO₄, filtered and concentrated to dryness to afford the title compound (813 mg, 63% yield).

### Intermediate 2A: N-Methyl-N-(3-(methylsulfonyl)benzyl)piperidin-4-amine

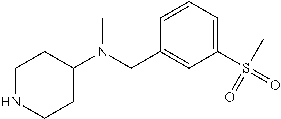 [SMILES: CN(Cc1cccc(S(C)(=O)=O)c1)C1CCNCC1]

Step 1. tert-Butyl 4-(methyl(3-(methylsulfonyl)benzyl)amino)piperidine-1-carboxylate: To a solution of tert-butyl 4-(methylamino)piperidine-1-carboxylate (1.0 g, 4.67 mmol) in DCM (5.6 mL), cooled at 0-5° C., 3-(methylsulfonyl)benzaldehyde (1.03 g, 5.60 mmol) and acetic acid (0.03 mL, 0.47 mmol) were added and the mixture was stirred at 0° C. for 30 min. Then, NaBH(OAc)₃ (1.48 g, 7.0 mmol) was added in three portions at 30 min intervals. The reaction mixture was further stirred at 0° c. for 30 min and finally it was stirred at r.t. overnight. Then, the reaction mixture was cooled with an ice-water bath, and aq. NaHCO₃ sat. sol. was added. It was extracted with DCM and the combined organic extracts were dried over MgSO₄, filtered and concentrated to dryness. The residue was purified by flash chromatography, silica gel, gradient DCM to MeOH:DCM (1:9), to give the title compound (1.08 g, 61% yield).

Step 2. Title compound: A solution of the compound obtained in Step 1 (200 mg, 0.52 mmol) and TFA (0.2 mL, 3.0 mmol) in DCM (5 mL) was stirred at r.t. overnight. The solvent was evaporated and the residue was partitioned between DCM and 1 N aq. NaOH solution. The phases were separated and the organic phase was dried over MgSO₄, filtered and concentrated to dryness to afford the title compound (133 mg, 90% yield).

This method was used for the preparation of Intermediates 2B-2E using suitable starting materials:

| INT | Structure | Chemical name |
|---|---|---|
| 2B | 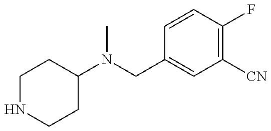 [SMILES: CN(Cc1ccc(F)c(C#N)c1)C1CCNCC1] | 2-fluoro-5- ((methyl(piperidin- 4-yl)amino)meth- yl)benzonitrile |
| 2C | 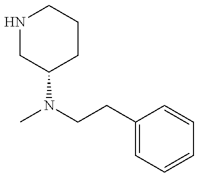 [SMILES: CN(CCc1ccccc1)[C@H]1CCCNC1] | (S)-N-methyl-N- phenethylpiperidin- 3-amine |
| 2D | 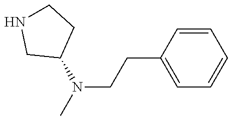 | (S)-N-methyl-N- phenethylpyrroli- din-3-amine |
| 2E | 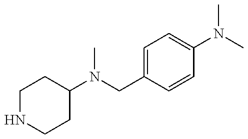 [SMILES: CN(C)c1ccc(CN(C)C2CCNCC2)cc1] | N-(4- (dimethyl- amino)benzyl)- N-methylpiperidin- 4-amine |

### Intermediate 2F: N,N-Dimethyl-3-((methyl(piperidin-4-yl)amino)methyl) benzamide

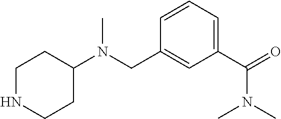 [SMILES: CN(C)C(=O)c1cccc(CN(C)C2CCNCC2)c1]

Step 1. tert-Butyl 4-((3-(dimethylcarbamoyl)benzyl)(methyl)amino)piperidine-1-carboxylate: A suspension of tert-butyl 4-(methylamino)piperidine-1-carboxylate (0.5 g, 2.33 mmol), 3-(chloromethyl)-N,N-dimethylbenzamide (0.46 g, 2.33 mmol) and K₂CO₃ (0.32 g, 2.33 mmol) in DMF (5 mL) was stirred at r.t. overnight. The solvent was evaporated and the crude was partitioned between water and EtOAc. The phases were separated and the aqueous phase was extracted with EtOAc. The combined organic extracts were washed with brine, dried over MgSO₄, filtered and concentrated to dryness to afford a residue that was purified by flash chromatography, silica gel, gradient DCM to MeOH:DCM (1:9), to give the title compound (565 mg, 64% yield).

Step 2. Title compound: Following the experimental procedure described in Step 2 of Intermediate 2A, starting from the product obtained in Step 1 (200 mg, 0.53 mmol), the title compound was obtained (116 mg, 79% yield).

### Intermediate 2G: N-Benzyl-N-isopentylazepan-3-amine

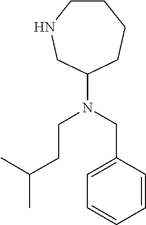 [SMILES: CC(C)CCN(Cc1ccccc1)C1CCCCNC1]

Step 1. tert-Butyl 3-(benzylamino)azepane-1-carboxylate: A solution of tert-butyl 3-aminoazepane-1-carboxylate (0.5 g, 2.33 mmol), benzaldehyde (0.17 mL, 2.33 mmol) and acetic acid (0.13 mL, 2.33 mmol) in DCE (5 mL) was stirred at r.t. for 30 min. Then, NaBH(OAc)₃ (0.742 g, 3.5 mmol) was added and the mixture was stirred at r.t. overnight. Aq. NaHCO₃ sat. sol. was added and it was extracted with DCM. The combined organic extracts were washed with aq. NaHCO₃ sat. sol. and brine, dried over MgSO₄, filtered and concentrated to dryness. The residue was purified by flash chromatography, silica gel, gradient DCM to MeOH:DCM (1:4), to give the title compound (446 mg, 63% yield).

Step 2. tert-Butyl 3-(benzyl(isopentyl)amino)azepane-1-carboxylate: Starting from the product obtained in Step 1 and following the experimental procedure described in Step 1 using 3-methylbutanal instead of benzaldehyde, the title compound was obtained (549 mg, quant. yield).

Step 3. Title compound: Following the experimental procedure described in Step 2 of Intermediate 2A, starting from the product obtained in Step 2 (549 mg, 1.47 mmol), the title compound was obtained (372 mg, 92% yield).

This method was used for the preparation of Intermediate 2H using suitable starting materials:

| INT | Structure | Chemical name |
|---|---|---|
| 2H | 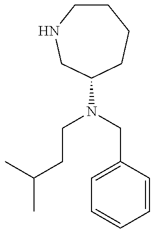 [SMILES: CC(C)CCN(Cc1ccccc1)[C@H]1CCCCNC1] | (S)-N-benzyl-N- isopentylazepan-3-amine |

### Intermediate 3A: (1r,4r)-N¹-Benzyl-N¹-methylcyclohexane-1,4-diamine dihydrochloride

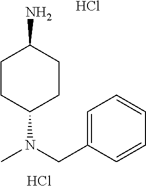 [SMILES: Cl ; CN(Cc1ccccc1)[C@H]1CC[C@H](N)CC1 ; Cl]

Step 1. tert-Butyl ((1r,4r)-4-(benzylamino)cyclohexyl)carbamate: A solution of tert-butyl ((1r,4r)-4-aminocyclohexyl)carbamate (0.5 g, 2.33 mmol), benzaldehyde (1.2 mL, 11.67 mmol) and acetic acid (0.13 mL, 2.33 mmol) in MeOH (15 mL) was stirred at r.t. overnight. Then, a mixture of NaBH₄ (0.88 g. 23.3 mmol) in MeOH (10 mL) was added and the reaction was stirred at r.t. for 1 h. The reaction mixture was then cooled to 0° C. and 10 wt % NaOH aq. sol. (10 mL) was added to quench the reaction. The solvent was evaporated and the resulting aqueous phase was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (0.48 g, 69% yield).

Step 2. tert-Butyl ((1r,4r)-4-(benzyl(methyl)amino)cyclohexyl)carbamate: A solution of the product obtained in Step 1 (0.48 g, 1.60 mmol), formaldehyde (1.48 mL, 16.0 mmol) and acetic acid (0.23 mL, 4.01 mmol) in MeOH (5 mL) was stirred at r.t. for 30 min. NaBH(OAc)₃ (0.85 g. 4.01 mmol) was added and the reaction mixture was stirred at r.t. overnight. Aq. NaHCO₃ sat. sol. was added and it was extracted with DCM. The combined organic extracts were washed with brine, dried over MgSO₄, filtered and concentrated to dryness to give the title compound (0.49 g, 98% yield).

Step 3. Title compound: To a solution of the product obtained in Step 2 (0.49 g, 1.56 mmol) in MeOH (36 mL), HCl solution (4 N in 1,4-dioxane, 1.95 mL, 7.82 mmol) was added. The reaction mixture was stirred at r.t. overnight and then it was concentrated to dryness. Additional HCl (4 N in 1,4-dioxane, 1.95 mL, 7.82 mmol) and MeOH (36 mL) were added to the residue and the mixture was stirred at r.t. for 2 days. The solvent was concentrated to dryness to give the title compound (0.44 g, 97% yield).

This method was used for the preparation of Intermediate 3B using suitable starting materials:

| Intermediate | Structure | Chemical name |
|---|---|---|
| 3B | 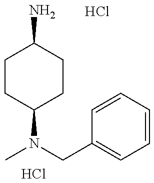 [SMILES: CN(Cc1ccccc1)[C@H]1CC[C@@H](N)CC1 ; Cl ; Cl] | (1s,4s)-N¹-benzyl-N¹- methylcyclohexane-1,4- diamine dihydrochloride |

### Intermediate 4A: 5-Chloro-3,3-dimethyl-2,3-dihydro-1H-pyrrolo[3,2-b]pyridine

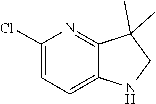

Step 1. 6-Chloro-2-iodo-N-(2-methylallyl)pyridin-3-amine: To a solution of 6-chloro-2-iodopyridin-3-amine (1.5 g, 5.9 mmol) in dry THF (34 mL), potassium tert-butoxide (0.79 g, 7.1 mmol) was added and the mixture was stirred at r.t. for 15 min. 3-Bromo-2-methyl-1-propene (0.73 mL, 7.1 mmol) was slowly added and the reaction mixture was stirred at r.t. for 2.5 days. Then, it was concentrated to dryness and the residue was diluted with water and DCM. The layers were separated and the aqueous phase was back extracted with DCM. The combined organic phases were dried over MgSO₄, filtered and concentrated under vacuum. The residue was purified by flash chromatography, silica gel, gradient CH to EtOAc, to give the title compound (1.31 g, 72% yield).

Step 2. 5-Chloro-3,3-dimethyl-2,3-dihydro-1H-pyrrolo[3,2-b]pyridine: A mixture of the product obtained in Step 1 (1.31 g, 4.25 mmol), tetrabutylammonium chloride (1.4 g, 5.1 mmol), TEA (1.77 mL, 12.7 mmol) and sodium formate (0.35 g, 5.1 mmol) in a mixture of DMSO (30 mL) and water (1.3 mL) was degassed by bubbling N₂ gas through the mixture. Palladium(II) acetate (0.143 g, 0.64 mmol) was added and the mixture was heated at 120° C. for 1 h under a N₂ atmosphere. After cooling, the solids were filtered off and the filtrate was diluted with water and EtOAc. The phases were separated and the aqueous phase was back extracted with EtOAc (×3). The combined organic phases were washed with water (×4), dried over MgSO₄, filtered and concentrated to dryness. The residue was purified by flash chromatography, silica gel, gradient CH to EtOAc, to give the title compound (450 mg, 58% yield).

This method was used for the preparation of Intermediates 4B-4C using suitable starting materials:

| INT | Structure | Chemical name |
|---|---|---|
| 4B | 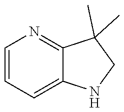 | 3,3-dimethyl-2,3-dihydro- 1H-pyrrolo[3,2-b]pyridine |
| 4C | 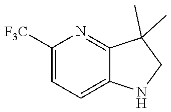 | 3,3-dimethyl-5- (trifluoromethyl)-2,3- dihydro-1H-pyrrolo[3,2- b]pyridine |

### Intermediate 4D: 3,3,5-Trimethyl-2,3-dihydro-1H-pyrrolo[3,2-b]pyridine

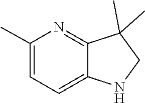

A mixture of Intermediate 4A (0.45 g, 2.46 mmol), trimethylboroxine (0.31 g, 2.46 mmol), K₂CO₃ (1.02 g, 7.39 mmol) and dichloro 1,1′-bis(diphenylphosphino)ferrocene palladium(II) dichloromethane adduct (9.9 mg, 0.135 mmol) in DME (15 mL) was placed in a microwave vial. The system was purged with vacuum/Ar cycles and it was irradiated under microwave heating at 120° C. for 1 h. After cooling, the solids were filtered off and the filtrate was concentrated to dryness. The residue was purified by flash chromatography, silica gel, gradient DCM to MeOH:DCM (1:4) to give the title compound (294 mg, 73% yield).

Alternatively, Intermediate 4D has also been prepared following the procedure described above for Intermediate 4A.

### Intermediate 4E: 5-Methoxy-3,3-dimethyl-2,3-dihydro-1H-pyrrolo[3,2-b]pyridine

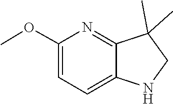

To a solution of Intermediate 4A (487 mg, 2.67 mmol) in DMF (10.6 mL), sodium methoxide solution (25 wt % in MeOH, 6.1 mL, 26.7 mmol) and copper(I) bromide (765 mg, 5.33 mmol) were added. The mixture was heated at 140° C. for 2 h in a sealed tube. After cooling to r.t., water and aq. NaHCO₃ sat. sol. were added and the phases were separated. The aqueous phase was extracted with EtOAc. The combined organic phases were dried over MgSO₄, filtered and concentrated to dryness. The residue was purified by flash chromatography, silica gel, gradient CH to EtOAc, to give the title compound (218 mg, 46% yield).

### Intermediate 4F: 3,3-Dimethyl-2,3-dihydro-1H-pyrrolo[3,2-b]pyridine-5-carbonitrile

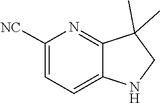

A mixture of Intermediate 4A (300 mg, 1.64 mmol), SPhos (67 mg, 0.164 mmol), tris(dibenzylideneacetone)dipalladium(0) (75 mg, 0.082 mmol) and zinc cyanide (289 mg, 2.46 mmol) in DMF (6.5 mL) was placed in a microwave vial. The system was inertized with Ar and it was irradiated under microwave heating at 150° C. for 35 min. Additional tris(dibenzylideneacetone)dipalladium(0) (75 mg, 0.082 mmol) was added and the mixture was irradiated again under microwave heating at 150° C. for 35 min. After cooling down to r.t., aq. NH₄Cl sat. sol. and EtOAc were added. The phases were separated and the aqueous phase was extracted with EtOAc. The combined organic phases were dried over MgSO₄, filtered and concentrated to dryness. The residue was purified by flash chromatography, silica gel, gradient CH to EtOAc, to give the title compound (123 mg, 43% yield).

### Intermediate 4G: 6-Fluoro-3,3-dimethyl-2,3-dihydro-1H-pyrrolo[3,2-b]pyridine

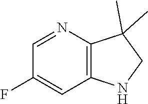

Step 1. 2-(3,5-Difluoropyridin-2-yl)-2-methylpropanenitrile: To a solution of 2,3,5-trifluoropyridine (8.0 g, 60.1 mmol) and isobutyronitrile (10.8 mL, 120 mmol) in toluene (20 mL), cooled at 0° C., sodium bis(trimethylsilylamide) solution (1.9 M in THF, 31.6 mL, 60.1 mmol) was added dropwise and the reaction mixture was stirred at r.t. overnight. It was concentrated to dryness and re-dissolved in EtOAc. The organic phase was washed with aq. NH₄Cl sat. sol., water and brine, dried over MgSO₄, filtered and concentrated to dryness. The residue was purified by flash chromatography, silica gel, gradient CH to EtOAc, to give the title compound (4.5 g, 41% yield).

Step 2. 2-(3,5-Difluoropyridin-2-yl)-2-methylpropan-1-amine: To a solution of the product obtained in Step 1 (4.5 g, 25.03 mmol) in MeOH (100 mL), cooled at 0° C., cobalt(II) chloride hexahydrate (2.98 g, 12.52 mmol) was added. Then, NaBH₄ (4.74 g, 125 mmol) was added and the reaction mixture was stirred at r.t. overnight. The mixture was cooled to 0° C., conc. ammonia (40 mL) was slowly added and it was stirred at 0° C. for 30 min. The heterogeneous mixture was filtered over a pad of Celite that was washed with MeOH. The filtrate was evaporated and the residue thus obtained was diluted with water and conc. ammonia. The aqueous phase was extracted with EtOAc and the combined organic extracts were washed with water and brine, dried over MgSO₄, filtered and concentrated to dryness to give the title compound (3.6 g, 77% yield).

Step 3. Title compound: In 3 separate microwave vials, the product obtained in Step 2 (1.2 g, 6.4 mmol, each vial) and K₂CO₃ (4 g, 28.9 mmol, each vial) were suspended in DMSO (8 mL, each vial). The reaction mixture was irradiated under microwave heating at 150° C. for 40 min. The reaction mixtures were combined, poured onto water and extracted with EtOAc. The combined organic extracts were washed with water and brine, dried over MgSO₄, filtered and concentrated to dryness. The residue was purified by flash chromatography, silica gel, gradient CH to EtOAc, to give the title compound (1.35 g, 42% yield).

### Intermediate 4H: 6-Fluoro-3,3,5-trimethyl-2,3-dihydro-1H-pyrrolo[3,2-b]pyridine

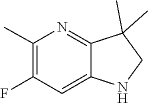

Step 1. 5-Bromo-6-fluoro-3,3-dimethyl-2,3-dihydro-1H-pyrrolo[3,2-b]pyridine: To a solution of Intermediate 4G (1.4 g, 8.75 mmol) in ACN (50 mL), cooled at 0° C., N-bromosuccinimide (779 mg, 4.38 mmol) was added portionwise. The reaction was stirred at 0° C. for 1 h. Then it was diluted with EtOAc and the organic phase was washed with brine, dried over MgSO₄, filtered and concentrated to dryness to give the title compound as a crude product (1.56 g, 74% yield). 1.2 g of the crude product were purified by flash chromatography, silica gel, gradient CH to EtOAc to give the title compound in higher purity (0.7 g, 42% yield).

Step 2. Title compound: Following the experimental procedure described in Intermediate 4D, starting from the product obtained in Step 1 (688 mg, 2.81 mmol), the title compound was obtained (258 mg, 51% yield).

### Intermediate 4I: 5-Methyl-2,3-dihydro-1H-pyrrolo[3,2-b]pyridine

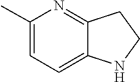

Step 1. tert-Butyl 5-methyl-1H-pyrrolo[3,2-b]pyridine-1-carboxylate: To a solution of 5-methyl-1H-pyrrolo[3,2-b]pyridine (375 mg, 2.84 mmol) in DCM (5.7 mL), cooled at 0° C., TEA (0.59 mL, 4.26 mmol) and a solution of di-tert-butyl dicarbonate (0.68 g, 3.12 mmol) in DCM (5.7 mL) were sequentially added and the mixture was stirred at r.t. overnight. Then, additional di-tert-butyl dicarbonate (0.27 g, 1.26 mmol) was added, the reaction mixture was left at r.t. for 4 h, and finally another portion of di-tert-butyl dicarbonate (0.27 g, 1.26 mmol) was added. The reaction mixture was stirred overnight. Water was added, the layers were separated and the aqueous phase was back extracted with DCM. The combined organic phases were washed with brine, dried over MgSO₄, filtered and concentrated under vacuum to give the title compound (865 mg, overweight, estimated 76 wt %; quant. yield was assumed).

Step 2. tert-Butyl 5-methyl-2,3-dihydro-1H-pyrrolo[3,2-b]pyridine-1-carboxylate: A mixture of the product obtained in Step 1 (865 mg, 2.83 mmol, 76 wt %) and palladium hydroxide (86 mg, 20 wt % on carbon, wet) in EtOH (11 mL) was stirred under 2 bars of H₂ at 60° C. for 1 day. The catalyst was filtered off and the solvent was removed under vacuum. The residue was purified by flash chromatography, silica gel, gradient CH to EtOAc, to give the title compound (464 mg, 70% yield).

Step 3. Title compound: To a solution of the product obtained in Step 2 (464 mg, 1.98 mmol) in a mixture of MeOH (2.1 mL) and 1,4-dioxane (0.5 mL), HCl solution (4 M in 1,4-dioxane, 2.1 mL, 8.36 mmol) was carefully added and the mixture was stirred at r.t. overnight. It was then concentrated to dryness and the residue was dissolved in water. The pH was made basic with 1 N aq. NaOH solution and it was extracted with DCM. The combined organic phases were dried over MgSO₄, filtered and concentrated under vacuum to yield the title compound (245 mg, 92% yield).

### Intermediate 5: (1r,4r)-4-((3,5-Difluorobenzyl)(methyl)amino)cyclohexane-1-carboxylic acid

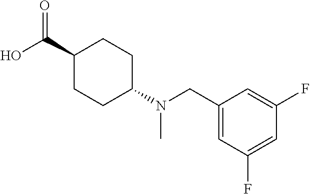 [SMILES: CN(Cc1cc(F)cc(F)c1)[C@H]1CC[C@H](C(=O)O)CC1]

A solution of (1r,4r)-4-aminocyclohexane-1-carboxylic acid hydrochloride (0.5 g, 2.78 mmol) and 3,5-difluorobenzaldehyde (0.29 mL, 3.0 mmol) in DMA (10 mL) was stirred at r.t. for 15 min. NaBH(OAc)₃ (0.88 g. 4.17 mmol) was added and the reaction mixture was stirred at r.t. for 2 h. After this time, formaldehyde (0.42 mL, 5.56 mmol) was added the reaction mixture was stirred at r.t. for 15 min. NaBH(OAc)₃ (0.88 g. 4.17 mmol) was added and the reaction mixture was stirred at r.t. during 16 h. Water was added and the mixture was extracted with DCM. The aqueous layer was acidified until pH=2, the excess of water was removed under reduced pressure and the crude was dried under vacuum at 45° C. overnight. The residue was purified by flash chromatography, silica gel, gradient DCM to MeOH, to give the title compound (0.44 g, 56% yield).

### SYNTHESIS OF EXAMPLES

### Example 1: N-(1-Benzylpiperidin-4-yl)-3,3,5-trimethyl-2,3-dihydro-1H-pyrrolo[3,2-b]pyridine-1-carboxamide

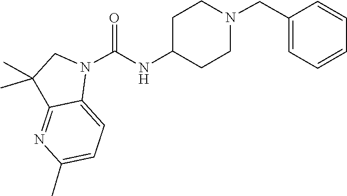 [SMILES: Cc1ccc2c(n1)C(C)(C)CN2C(=O)NC1CCN(Cc2ccccc2)CC1]

To a solution of bis(trichloromethyl) carbonate (44 mg, 0.148 mmol) in DCM (2.7 mL), cooled at 0° C., a solution of Intermediate 4D (60 mg, 0.37 mmol) and DIPEA (0.13 mL, 0.74 mmol) in DCM (2 mL) was added dropwise. The reaction mixture was stirred at 0° C. for 30 min. Then, a solution of 1-benzylpiperidin-4-amine (70 mg, 0.37 mmol) and DIPEA (0.129 mL, 0.74 mmol) in DMF (1 mL) was added. The reaction mixture was stirred at 0° C. for 5 min and at r.t. for 1 h. It was diluted with MeOH (2 mL) to quench the reaction and finally the solvent was evaporated. The residue was partitioned between aq. NaHCO₃ sat. solution and EtOAc. The phases were separated and the aqueous phase was extracted with EtOAc. The combined organic extracts were dried over MgSO₄, filtered and concentrated to dryness. The residue was purified by flash chromatography, silica gel, gradient DCM to MeOH:DCM (1:4), to give the title compound (71 mg, 51% yield).

HPLC retention time (Method A): 4.59 min; MS: 379.2 (M+H).

This method was used for the preparation of Examples 2-78 using suitable starting materials:

| | | | Ret | | |
|---|---|---|---|---|---|
| | | | time | MS | |
| EX | Structure | Chemical name | (min) | (M + H) | Method |
| 2 | 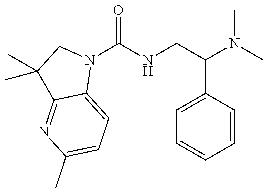 | N-(2-(dimethylamino)-2-phenylethyl)- 3,3,5-trimethyl-2,3-dihydro-1H- pyrrolo[3,2-b]pyridine-1-carboxamide | 4.16 | 353.2 | A |
| 3 | 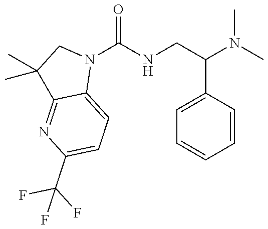 | N-(2-(dimethylamino)-2-phenylethyl)-3,3- dimethyl-5-(trifluoromethyl)-2,3-dihydro- 1H-pyrrolo[3,2-b]pyridine-1-carboxamide | 5.27 | 407.2 | A |
| 4 | 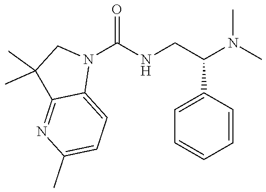 | (R)-N-(2-(dimethylamino)-2-phenylethyl)- 3,3,5-trimethyl-2,3-dihydro-1H- pyrrolo[3,2-b]pyridine-1-carboxamide | 4.19 | 353.2 | A |
| 5 | 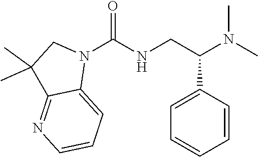 | (R)-N-(2-(dimethylamino)-2-phenylethyl)- 3,3-dimethyl-2,3-dihydro-1H-pyrrolo[3,2- b]pyridine-1-carboxamide | 3.98 | 339.2 | A |
| 6 | 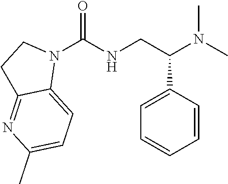 [SMILES: Cc1ccc2c(n1)CCN2C(=O)NC[C@@H](c1ccccc1)N(C)C] | (R)-N-(2-(dimethylamino)-2-phenylethyl)- 5-methyl-2,3-dihydro-1H-pyrrolo[3,2- b]pyridine-1-carboxamide | 3.70 | 325.2 | A |
| 7 | 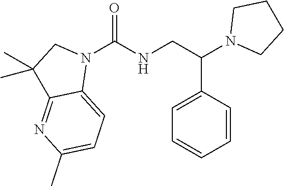 [SMILES: Cc1ccc2c(n1)C(C)(C)CN2C(=O)NCC(c1ccccc1)N1CCCC1] | 3,3,5-trimethyl-N-(2-phenyl-2-(pyrrolidin- 1-yl)ethyl)-2,3-dihydro-1H-pyrrolo[3,2- b]pyridine-1-carboxamide | 4.49 | 379.2 | A |
| 8 | 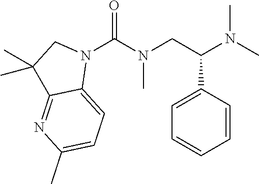 | (R)-N-(2-(dimethylamino)-2-phenylethyl)- N,3,3,5-tetramethyl-2,3-dihydro-1H- pyrrolo[3,2-b]pyridine-1-carboxamide | 4.29 | 367.2 | A |
| 9 | 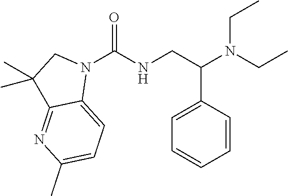 [SMILES: CCN(CC)C(CNC(=O)N1CC(C)(C)c2nc(C)ccc21)c1ccccc1] | N-(2-(diethylamino)-2-phenylethyl)-3,3,5- trimethyl-2,3-dihydro-1H-pyrrolo[3,2- b]pyridine-1-carboxamide | 5.04 | 381.2 | A |
| 10 | 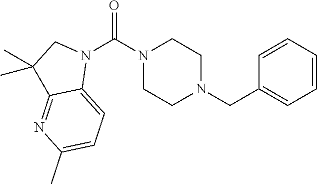 [SMILES: Cc1ccc2c(n1)C(C)(C)CN2C(=O)N1CCN(Cc2ccccc2)CC1] | (4-benzylpiperazin-1-yl)(3,3,5-trimethyl- 2,3-dihydro-1H-pyrrolo[3,2-b]pyridin-1- yl)methanone | 4.71 | 365.2 | A |
| 11 | 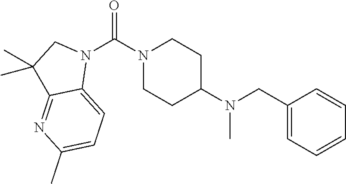 [SMILES: Cc1ccc2c(n1)C(C)(C)CN2C(=O)N1CCC(N(C)Cc2ccccc2)CC1] | (4-(benzyl(methyl)amino)piperidin-1- yl)(3,3,5-trimethyl-2,3-dihydro-1H- pyrrolo[3,2-b]pyridin-1-yl)methanone | 4.94 | 393.3 | A |
| 12 | 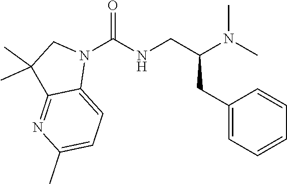 [SMILES: Cc1ccc2c(n1)C(C)(C)CN2C(=O)NC[C@H](Cc1ccccc1)N(C)C] | (S)-N-(2-(dimethylamino)-3- phenylpropyl)-3,3,5-trimethyl-2,3- dihydro-1H-pyrrolo[3,2-b]pyridine-1- carboxamide | 4.56 | 367.2 | A |
| 13 | 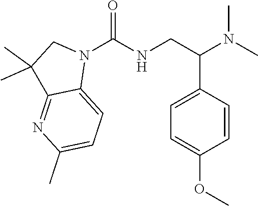 | N-(2-(dimethylamino)-2-(4- methoxyphenyl)ethyl)-3,3,5-trimethyl- 2,3-dihydro-1H-pyrrolo[3,2-b]pyridine-1- carboxamide | 4.15 | 383.2 | A |
| 14 | 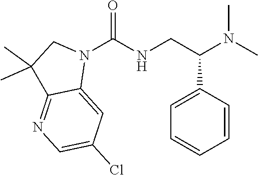 | (R)-6-chloro-N-(2-(dimethylamino)-2- phenylethyl)-3,3-dimethyl-2,3-dihydro- 1H-pyrrolo[3,2-b]pyridine-1-carboxamide | 4.81 | 373.1 | A |
| 15 | 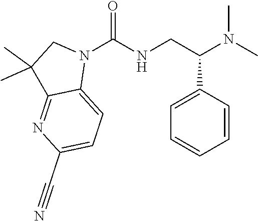 | (R)-5-cyano-N-(2-(dimethylamino)-2- phenylethyl)-3,3-dimethyl-2,3-dihydro- 1H-pyrrolo[3,2-b]pyridine-1-carboxamide | 4.56 | 364.2 | A |
| 16 | 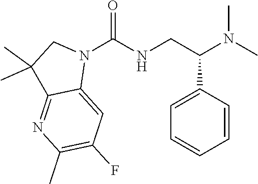 | (R)-N-(2-(dimethylamino)-2-phenylethyl)- 6-fluoro-3,3,5-trimethyl-2,3-dihydro-1H- pyrrolo[3,2-b]pyridine-1-carboxamide | 4.70 | 371.2 | A |
| 17 | 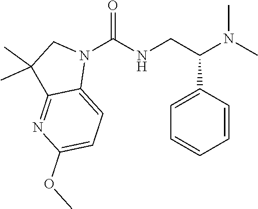 | (R)-N-(2-(dimethylamino)-2-phenylethyl)- 5-methoxy-3,3-dimethyl-2,3-dihydro-1H- pyrrolo[3,2-b]pyridine-1-carboxamide | 4.77 | 369.2 | A |
| 18 | 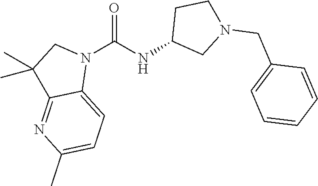 [SMILES: Cc1ccc2c(n1)C(C)(C)CN2C(=O)N[C@@H]1CCN(Cc2ccccc2)C1] | (R)-N-(1-benzylpyrrolidin-3-yl)-3,3,5- trimethyl-2,3-dihydro-1H-pyrrolo[3,2- b]pyridine-1-carboxamide | 4.50 | 365.2 | A |
| 19 | 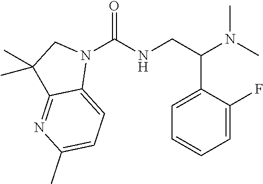 [SMILES: Cc1ccc2c(n1)C(C)(C)CN2C(=O)NCC(c1ccccc1F)N(C)C] | N-(2-(dimethylamino)-2-(2- fluorophenyl)ethyl)-3,3,5-trimethyl-2,3- dihydro-1H-pyrrolo[3,2-b]pyridine-1- carboxamide | 4.30 | 371.2 | A |
| 20 | 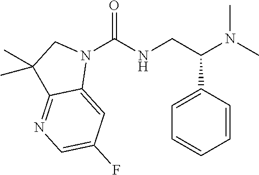 | (R)-N-(2-(dimethylamino)-2-phenylethyl)- 6-fluoro-3,3-dimethyl-2,3-dihydro-1H- pyrrolo[3,2-b]pyridine-1-carboxamide | 4.46 | 357.1 | A |
| 21 | 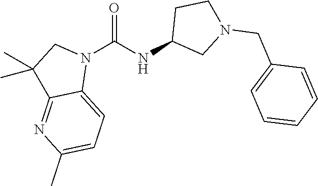 [SMILES: Cc1ccc2c(n1)C(C)(C)CN2C(=O)N[C@H]1CCN(Cc2ccccc2)C1] | (S)-N-(1-benzylpyrrolidin-3-yl)-3,3,5- trimethyl-2,3-dihydro-1H-pyrrolo[3,2- b]pyridine-1-carboxamide | 4.48 | 365.2 | A |
| 22 | 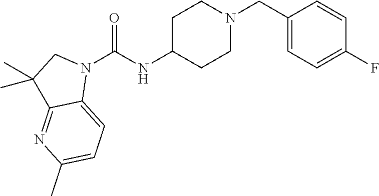 [SMILES: Cc1ccc2c(n1)C(C)(C)CN2C(=O)NC1CCN(Cc2ccc(F)cc2)CC1] | N-(1-(4-fluorobenzyl)piperidin-4-yl)- 3,3,5-trimethyl-2,3-dihydro-1H- pyrrolo[3,2-b]pyridine-1-carboxamide | 4.69 | 397.2 | A |
| 23 | 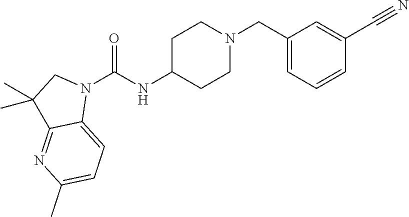 [SMILES: Cc1ccc2c(n1)C(C)(C)CN2C(=O)NC1CCN(Cc2cccc(C#N)c2)CC1] | N-(1-(3-cyanobenzyl)piperidin-4-yl)- 3,3,5-trimethyl-2,3-dihydro-1H- pyrrolo[3,2-b]pyridine-1-carboxamide | 4.48 | 404.2 | A |
| 24 | 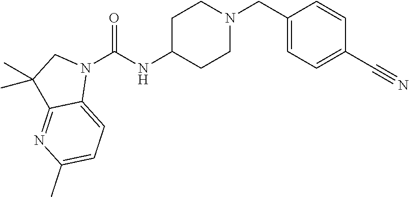 [SMILES: Cc1ccc2c(n1)C(C)(C)CN2C(=O)NC1CCN(Cc2ccc(C#N)cc2)CC1] | N-(1-(4-cyanobenzyl)piperidin-4-yl)- 3,3,5-trimethyl-2,3-dihydro-1H- pyrrolo[3,2-b]pyridine-1-carboxamide | 4.51 | 404.2 | A |
| 25 | 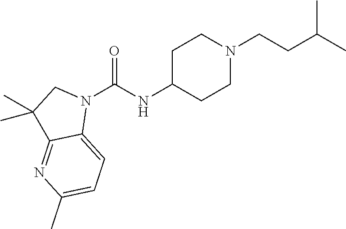 [SMILES: Cc1ccc2c(n1)C(C)(C)CN2C(=O)NC1CCN(CCC(C)C)CC1] | N-(1-isopentylpiperidin-4-yl)-3,3,5- trimethyl-2,3-dihydro-1H-pyrrolo[3,2- b]pyridine-1-carboxamide | 4.46 | 359.3 | A |
| 26 | 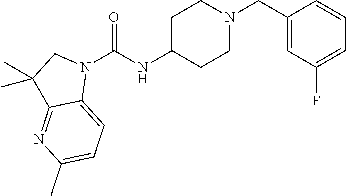 [SMILES: Cc1ccc2c(n1)C(C)(C)CN2C(=O)NC1CCN(Cc2cccc(F)c2)CC1] | N-(1-(3-fluorobenzyl)piperidin-4-yl)- 3,3,5-trimethyl-2,3-dihydro-1H- pyrrolo[3,2-b]pyridine-1-carboxamide | 4.80 | 397.2 | A |
| 27 | 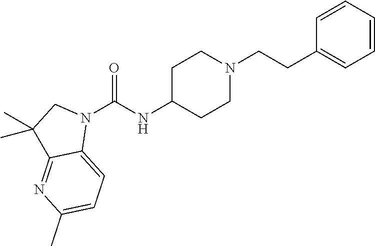 [SMILES: Cc1ccc2c(n1)C(C)(C)CN2C(=O)NC1CCN(CCc2ccccc2)CC1] | 3,3,5-trimethyl-N-(1-phenethyl)piperidin- 4-yl)-2,3-dihydro-1H-pyrrolo[3,2- b]pyridine-1-carboxamide | 4.71 | 393.2 | A |
| 28 | 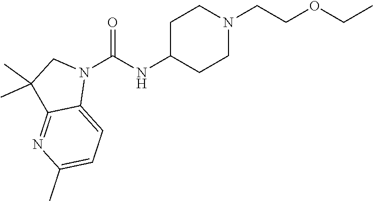 [SMILES: CCOCCN1CCC(NC(=O)N2CC(C)(C)c3nc(C)ccc32)CC1] | N-(1-(2-ethoxyethyl)piperidin-4-yl)-3,3,5- trimethyl-2,3-dihydro-1H-pyrrolo[3,2- b]pyridine-1-carboxamide | 3.75 | 361.2 | A |
| 29 | 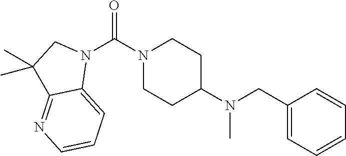 [SMILES: CN(Cc1ccccc1)C1CCN(C(=O)N2CC(C)(C)c3ncccc32)CC1] | (4-(benzyl(methyl)amino)piperidin-1- yl)(3,3-dimethyl-2,3-dihydro-1H- pyrrolo[3,2-b]pyridin-1-yl)methanone | 4.69 | 379.2 | A |
| 30 | 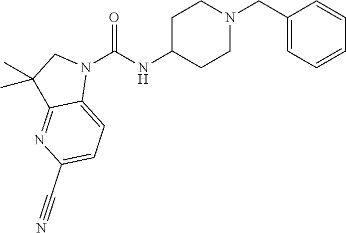 [SMILES: CC1(C)CN(C(=O)NC2CCN(Cc3ccccc3)CC2)c2ccc(C#N)nc21] | N-(1-benzylpiperidin-4-yl)-5-cyano-3,3- dimethyl-2,3-dihydro-1H-pyrrolo[3,2- b]pyridine-1-carboxamide | 4.97 | 390.2 | A |
| 31 | 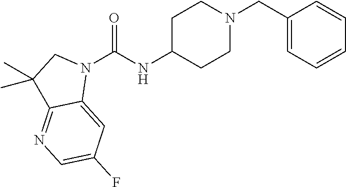 [SMILES: CC1(C)CN(C(=O)NC2CCN(Cc3ccccc3)CC2)c2cc(F)cnc21] | N-(1-benzylpiperidin-4-yl)-6-fluoro-3,3- dimethyl-2,3-dihydro-1H-pyrrolo[3,2- b]pyridine-1-carboxamide | 4.90 | 383.2 | A |
| 32 | 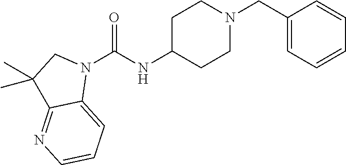 [SMILES: CC1(C)CN(C(=O)NC2CCN(Cc3ccccc3)CC2)c2cccnc21] | N-(1-benzylpiperidin-4-yl)-3,3-dimethyl- 2,3-dihydro-1H-pyrrolo[3,2-b]pyridine-1- carboxamide | 4.40 | 365.2 | A |
| 33 | 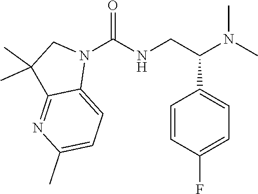 | (R)-N-(2-(dimethylamino)-2-(4- fluorophenyl)ethyl)-3,3,5-trimethyl-2,3- dihydro-1H-pyrrolo[3,2-b]pyridine-1- carboxamide | 4.35 | 371.2 | A |
| 34 | 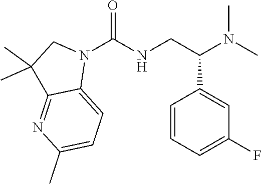 [SMILES: Cc1ccc2c(n1)C(C)(C)CN2C(=O)NC[C@@H](c1cccc(F)c1)N(C)C] | (R)-N-(2-(dimethylamino)-2-(3- fluorophenyl)ethyl)-3,3,5-tyrimethyl-2,3- dihydro-1H-pyrrolo[3,2-b]pyridine-1- carboxamide | 4.36 | 371.2 | A |
| 35 | 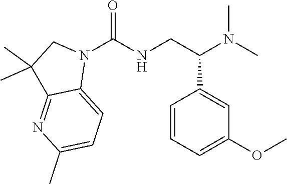 | (R)-N-(2-(dimethylamino)-2-(3- methoxyphenyl)ethyl)-3,3,5-trimethyl- 2,3-dihydro-1H-pyrrolo[3,2-b]pyridine-1- carboxamide | 4.25 | 383.2 | A |
| 36 | 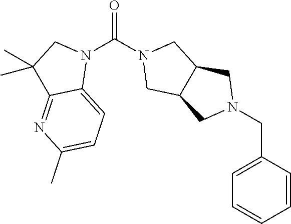 [SMILES: Cc1ccc2c(n1)C(C)(C)CN2C(=O)N1C[C@H]2CN(Cc3ccccc3)C[C@H]2C1] | ((3aR,6aS)-5- benzylhexahydropyrrolo[3,4-c]pyrrol- 2(1H)-yl)(3,3,5-trimethyl-2,3-dihydro-1H- pyrrolo[3,2-b]pyridin-1-yl)methanone | 4.86 | 391.2 | A |
| 37 | 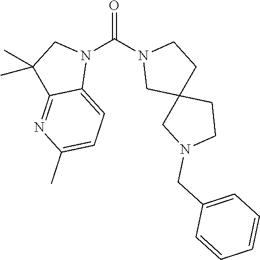 [SMILES: Cc1ccc2c(n1)C(C)(C)CN2C(=O)N1CCC2(CCN(Cc3ccccc3)C2)C1] | (7-benzyl-2,7-diazaspiro[4.4]nonan-2- yl)(3,3,5-trimethyl-2,3-dihydro-1H- pyrrolo[3,2-b]pyridin-1-yl)methanone | 4.84 | 405.2 | A |
| 38 | 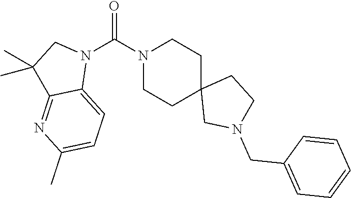 [SMILES: Cc1ccc2c(n1)C(C)(C)CN2C(=O)N1CCC2(CCN(Cc3ccccc3)C2)CC1] | (2-benzyl-2,8-diazaspiro[4.5]decan-8- yl)(3,3,5-trimethyl-2,3-dihydro-1H- pyrrolo[3,2-b]pyridin-1-yl)methanone | 5.17 | 419.3 | A |
| 39 | 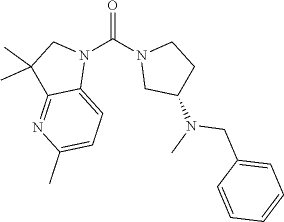 [SMILES: Cc1ccc2c(n1)C(C)(C)CN2C(=O)N1CC[C@H](N(C)Cc2ccccc2)C1] | (S)-(3-(benzyl(methyl)amino)pyrrolidin-1- yl)(3,3,5-trimethyl-2,3-dihydro-1H- pyrrolo[3,2-b]pyridin-1-yl)methanone | 4.74 | 379.2 | A |
| 40 | 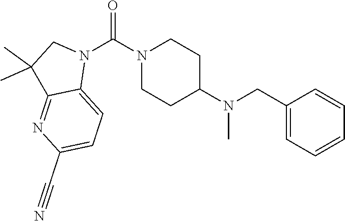 [SMILES: CN(Cc1ccccc1)C1CCN(C(=O)N2CC(C)(C)c3nc(C#N)ccc32)CC1] | 1-(4-(benzyl(methyl)amino)piperidine-1- carbonyl)-3,3-dimethyl-2,3-dihydro-1H- pyrrolo[3,2-b]pyridine-5-carbonitrile | 5.19 | 404.2 | A |
| 41 | 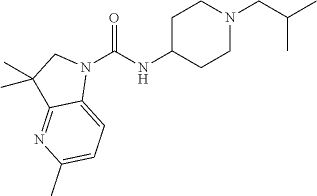 [SMILES: Cc1ccc2c(n1)C(C)(C)CN2C(=O)NC1CCN(CC(C)C)CC1] | N-(1-isobutylpiperidin-4-yl)-3,3,5- trimethyl-2,3-dihydro-1H-pyrrolo[3,2- b]pyridine-1-carboxamide | 4.20 | 345.2 | A |
| 42 | 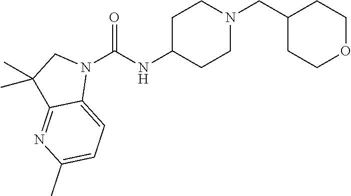 [SMILES: Cc1ccc2c(n1)C(C)(C)CN2C(=O)NC1CCN(CC2CCOCC2)CC1] | 3,3,5-trimethyl-N-(1-((tetrahydro-2H- pyran-4-yl)methyl)piperidin-4-yl)-2,3- dihydro-1H-pyrrolo[3,2-b]pyridine-1- carboxamide | 3.76 | 387.3 | A |
| 43 | 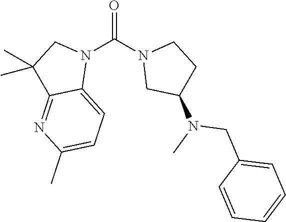 [SMILES: Cc1ccc2c(n1)C(C)(C)CN2C(=O)N1CC[C@@H](N(C)Cc2ccccc2)C1] | (R)-(3-(benzyl(methyl)amino)pyrrolidin-1- yl)(3,3,5-trimethyl-2,3-dihydro-1H- pyrrolo[3,2-b]pyridin-1-yl)methanone | 4.75 | 379.2 | A |
| 44 | 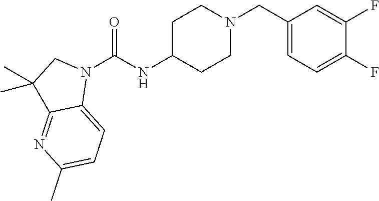 [SMILES: Cc1ccc2c(n1)C(C)(C)CN2C(=O)NC1CCN(Cc2ccc(F)c(F)c2)CC1] | N-(1-(3,4-difluorobenzyl)piperidin-4-yl)- 3,3,5-trimethyl-2,3-dihydro-1H- pyrrolo[3,2-b]pyridine-1-carboxamide | 4.90 | 415.2 | A |
| 45 | 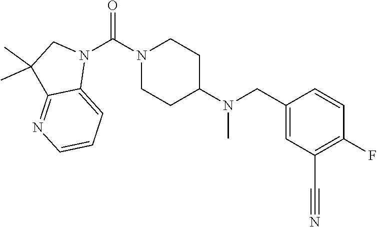 [SMILES: CN(Cc1ccc(F)c(C#N)c1)C1CCN(C(=O)N2CC(C)(C)c3ncccc32)CC1] | 5-(((1-(3,3-dimethyl-2,3-dihydro-1H- pyrrolo[3,2-b]pyridine-1- carbonyl)piperidin-4- yl)(methyl)amino)methyl)-2- fluorobenzonitrile | 4.77 | 422.2 | A |
| 46 | 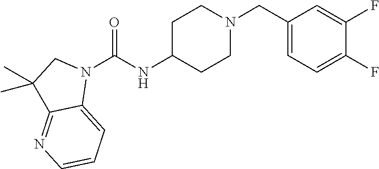 [SMILES: CC1(C)CN(C(=O)NC2CCN(Cc3ccc(F)c(F)c3)CC2)c2cccnc21] | N-(1-(3,4-difluorobenzyl)piperidin-4-yl)- 3,3-dimethyl-2,3-dihydro-1H-pyrrolo[3,2- b]pyridine-1-carboxamide | 4.70 | 401.2 | A |
| 47 | 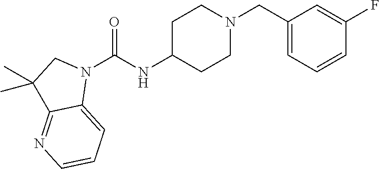 [SMILES: CC1(C)CN(C(=O)NC2CCN(Cc3cccc(F)c3)CC2)c2cccnc21] | N-(1-(3-(fluorobenzyl)piperidin-4-yl)-3,3- dimethyl-2,3-dihydro-1H-pyrrolo[3,2- b]pyridine-1-carboxamide | 4.59 | 383.2 | A |
| 48 | 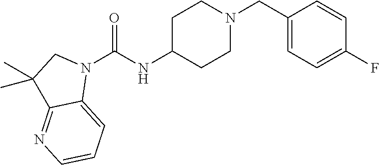 [SMILES: CC1(C)CN(C(=O)NC2CCN(Cc3ccc(F)cc3)CC2)c2cccnc21] | N-(1-(4-fluorobenzyl)piperidin-4-yl)-3,3- dimethyl-2,3-dihydro-1H-pyrrolo[3,2- b]pyridine-1-carboxamide | 4.51 | 383.2 | A |
| 49 | 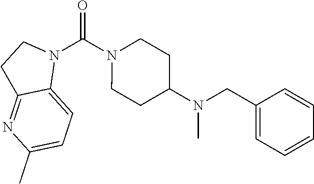 [SMILES: Cc1ccc2c(n1)CCN2C(=O)N1CCC(N(C)Cc2ccccc2)CC1] | (4-(benzyl(methyl)amino)piperidin-1- yl)(5-methyl-2,3-dihydro-1H-pyrrolo[3,2- b]pyridin-1-yl)methanone | 4.37 | 365.2 | A |
| 50 | 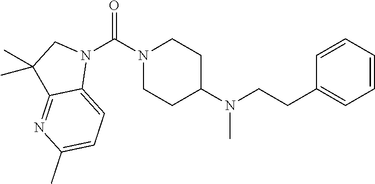 [SMILES: Cc1ccc2c(n1)C(C)(C)CN2C(=O)N1CCC(N(C)CCc2ccccc2)CC1] | (4-(methyl(phenethyl)amino)piperidin-1- yl)(3,3,5-trimethyl-2,3-dihydro-1H- pyrrolo[3,2-b]pyridin-1-yl)methanone | 4.94 | 407.3 | A |
| 51 | 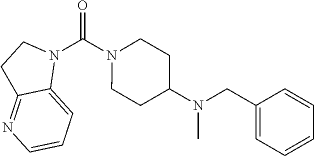 [SMILES: CN(Cc1ccccc1)C1CCN(C(=O)N2CCc3ncccc32)CC1] | (4-(benzyl(methyl)amino)piperidin-1- yl)(2,3-dihydro-1H-pyrrolo[3,2-b]pyridin- 1-yl)methanone | 4.17 | 351.0 | A |
| 52 | 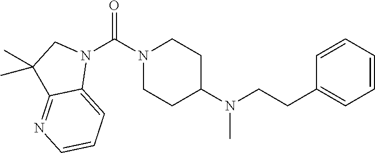 [SMILES: CN(CCc1ccccc1)C1CCN(C(=O)N2CC(C)(C)c3ncccc32)CC1] | (3,3-dimethyl-2,3-dihydro-1H-pyrrolo[3,2- b]pyridin-1-yl)(4- (methyl(phenethyl)amino)piperidin-1- yl)methanone | 4.71 | 393.0 | A |
| 53 | 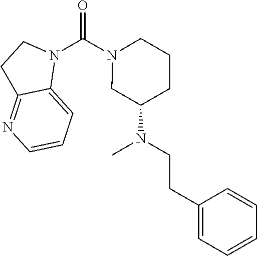 [SMILES: CN(CCc1ccccc1)[C@H]1CCCN(C(=O)N2CCc3ncccc32)C1] | (S)-(2,3-dihydro-1H-pyrrolo[3,2-b]pyridin- 1-yl)(3- (methyl(phenethyl)amino)piperidin-1- yl)methanone | 4.37 | 365.0 | A |
| 54 | 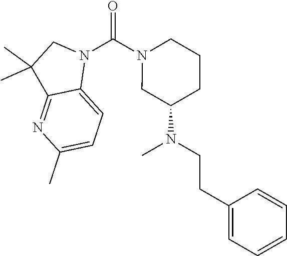 [SMILES: Cc1ccc2c(n1)C(C)(C)CN2C(=O)N1CCC[C@H](N(C)CCc2ccccc2)C1] | (S)-(3- (methyl(phenethyl)amino)piperidin-1- yl)(3,3,5-trimethyl-2,3-dihydro-1H- pyrrolo[3,2-b]pyridin-1-yl)methanone | 5.06 | 407.0 | A |
| 55 | 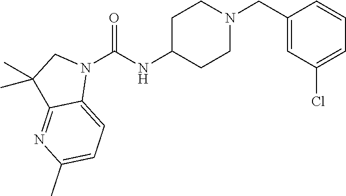 [SMILES: Cc1ccc2c(n1)C(C)(C)CN2C(=O)NC1CCN(Cc2cccc(Cl)c2)CC1] | N-(1-(3-chlorobenzyl)piperidin-4-yl)- 3,3,5-trimethyl-2,3-dihydro-1H- pyrrolo[3,2-b]pyridine-1-carboxamide | 5.04 | 413.1 | A |
| 56 | 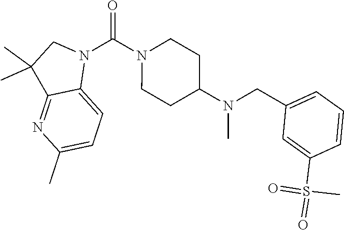 [SMILES: Cc1ccc2c(n1)C(C)(C)CN2C(=O)N1CCC(N(C)Cc2cccc(S(C)(=O)=O)c2)CC1] | (4-(methyl(3- (methylsulfonyl)benzyl)amino)piperidin- 1-yl)(3,3,5-trimethyl-2,3-dihydro-1H- pyrrolo[3,2-b]pyridin-1-yl)methanone | 4.27 | 471.1 | A |
| 57 | 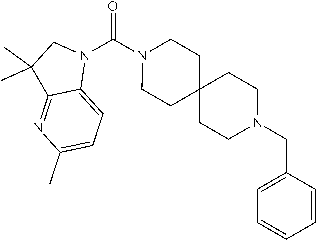 [SMILES: Cc1ccc2c(n1)C(C)(C)CN2C(=O)N1CCC2(CCN(Cc3ccccc3)CC2)CC1] | (9-benzyl-3,9-diazaspiro[5.5]undecan-3- yl)(3,3,5-trimethyl-2,3-dihydro-1H- pyrrolo[3,2-b]pyridin-1-yl)methanone | 5.10 | 433.2 | A |
| 58 | 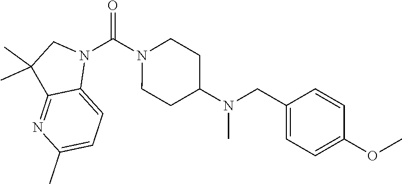 | (4-((4- methoxybenzyl)(methyl)amino)piperidin- 1-yl)(3,3,5-trimethyl-2,3-dihydro-1H- pyrrolo[3,2-b]pyridin-1-yl)methanone | 4.76 | 423.2 | A |
| 59 | 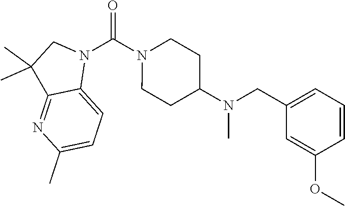 | (4-((3- methoxybenzyl)(methyl)amion)piperidin- 1-yl)(3,3,5-trimethyl-2,3-dihydro-1H- pyrrolo[3,2-b]pyridin-1-yl)methanone | 4.91 | 423.2 | A |
| 60 | 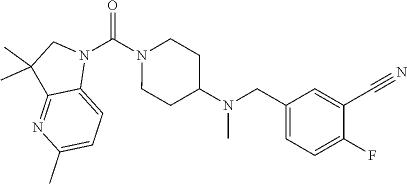 [SMILES: Cc1ccc2c(n1)C(C)(C)CN2C(=O)N1CCC(N(C)Cc2ccc(F)c(C#N)c2)CC1] | 2-fluoro-5-((methyl(1-(3,3,5-trimethyl- 2,3-dihydro-1H-pyrrolo[3,2-b]pyridine-1- carbonyl)piperidin-4- yl)amino)methylbenzonitrile | 5.00 | 436.1 | A |
| 61 | 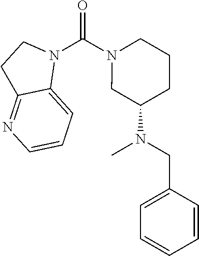 [SMILES: CN(Cc1ccccc1)[C@H]1CCCN(C(=O)N2CCc3ncccc32)C1] | (S)-(3-(benzyl(methyl)amino)piperidin-1- yl)(2,3-dihydro-1H-pyrrolo[3,2-b]pyridin- 1-yl)methanone | 4.38 | 351.1 | A |
| 62 | 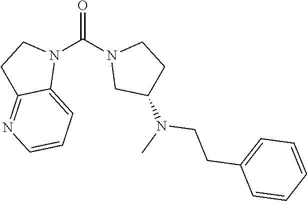 [SMILES: CN(CCc1ccccc1)[C@H]1CCN(C(=O)N2CCc3ncccc32)C1] | (S)-(2,3-dihydro-1H-pyrrolo[3,2-b]pyridin- 1-yl)(3- (methyl(phenethyl)amino)pyrrolidin-1- yl)methanone | 4.10 | 351.1 | A |
| 63 | 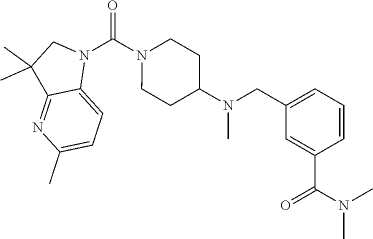 | N,N-dimethyl-3-((methyl(1-(3,3,5- trimethyl-2,3-dihydro-1H-pyrrolo[3,2- b]pyridine-1-carbonyl)piperidin-4- yl)amino)methyl)benzamide | 4.12 | 464.2 | A |
| 64 | 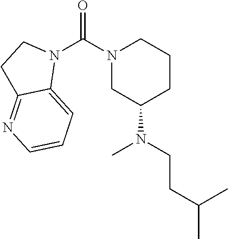 [SMILES: CC(C)CCN(C)[C@H]1CCCN(C(=O)N2CCc3ncccc32)C1] | (S)-(2,3-dihydro-1H-pyrrolo[3,2-b]pyridin- 1-yl)(3- (isopentyl(methyl)amino)piperidin-1- yl)methanone | 4.13 | 331.1 | A |
| 65 | 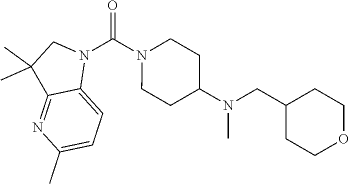 [SMILES: Cc1ccc2c(n1)C(C)(C)CN2C(=O)N1CCC(N(C)CC2CCOCC2)CC1] | (4-(methyl((tetrahydro-2H-pyran-4- yl)methyl)amino)piperidin-1-yl)(3,3,5- trimethyl-2,3-dihydro-1H-pyrrolo[3,2- b]pyridin-1-yl)methanone | 3.96 | 401.2 | A |
| 66 | 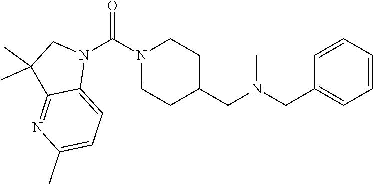 [SMILES: Cc1ccc2c(n1)C(C)(C)CN2C(=O)N1CCC(CN(C)Cc2ccccc2)CC1] | (4- ((benzyl(methyl)amino)methyl)piperidin- 1-yl)(3,3,5-trimethyl-2,3-dihydro-1H- pyrrolo[3,2-b]pyridin-1-yl)methanone | 5.56 | 407.2 | A |
| 67 | 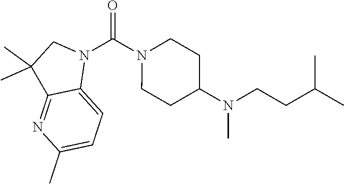 [SMILES: Cc1ccc2c(n1)C(C)(C)CN2C(=O)N1CCC(N(C)CCC(C)C)CC1] | (4-(isopentyl(methyl)amino)piperidin-1- yl)(3,3,5-trimethyl-2,3-dihydro-1H- pyrrolo[3,2-b]pyridin-1-yl)methanone | 4.49 | 373.2 | A |
| 68 | 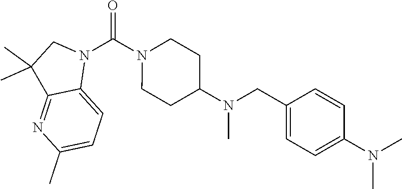 | (4-((4- (dimethylamino)benzyl)(methyl)amino)pi- peridin-1-yl)(3,3,5-trimethyl-2,3-dihydro- 1H-pyrrolo[3,2-b]pyridin-1-yl)methanone | 4.72 | 436.6 | A |
| 69 | 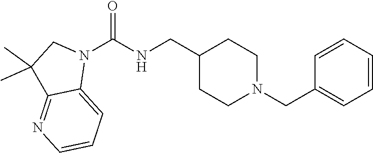 [SMILES: CC1(C)CN(C(=O)NCC2CCN(Cc3ccccc3)CC2)c2cccnc21] | N-((1-benzylpiperidin-4-yl)methyl)-3,3- dimethyl-2,3-dihydro-1H-pyrrolo[3,2- b]pyridine-1-carboxamide | 4.08 | 378.7 | A |
| 70 | 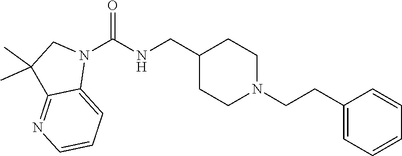 [SMILES: CC1(C)CN(C(=O)NCC2CCN(CCc3ccccc3)CC2)c2cccnc21] | 3,3-dimethyl-N-((1-phenethylpiperidin-4- yl)methyl)-2,3-dihydro-1H-pyrrolo[3,2- b]pyridine-1-carboxamide | 4.10 | 392.7 | A |
| 71 | 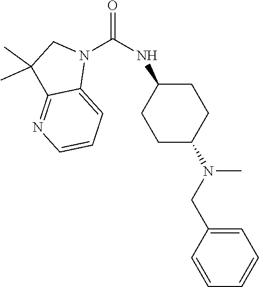 [SMILES: CN(Cc1ccccc1)[C@H]1CC[C@H](NC(=O)N2CC(C)(C)c3ncccc32)CC1] | N-((1r,4r)-4- (benzyl(methyl)amino)cyclohexyl)-3,3- dimethyl-2,3-dihydro-1H-pyrrolo[3,2- b]pyridine-1-carboxamide | 4.46 | 393.2 | A |
| 72 | 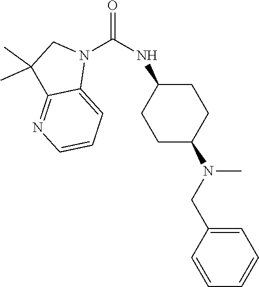 [SMILES: CN(Cc1ccccc1)[C@H]1CC[C@@H](NC(=O)N2CC(C)(C)c3ncccc32)CC1] | N-((1s,4s)-4- (benzyl(methyl)amino)cyclohexyl)-3,3- dimethyl-2,3-dihydro-1H-pyrrolo[3,2- b]pyridine-1-carboxamide | 4.78 | 393.3 | A |
| 73 | 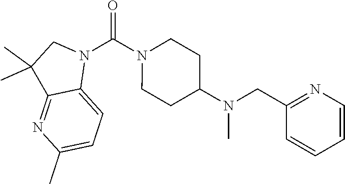 [SMILES: Cc1ccc2c(n1)C(C)(C)CN2C(=O)N1CCC(N(C)Cc2ccccn2)CC1] | (4-(methyl(pyridin-2- ylmethyl)amino)piperidin-1-yl)(3,3,5- trimethyl-2,3-dihydro-1H-pyrrolo[3,2- b]pyridin-1-yl)methanone | 3.95 | 394.2 | A |
| 74 | 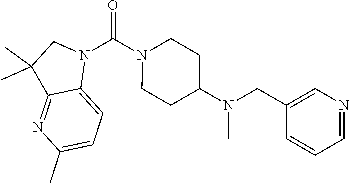 [SMILES: Cc1ccc2c(n1)C(C)(C)CN2C(=O)N1CCC(N(C)Cc2cccnc2)CC1] | (4-(methyl(pyridin-3- ylmethyl)amino)piperidin-1-yl)(3,3,5- trimethyl-2,3-dihydro-1H-pyrrolo[3,2- b]pyridin-1-yl)methanone | 3.89 | 394.2 | A |
| 75 | 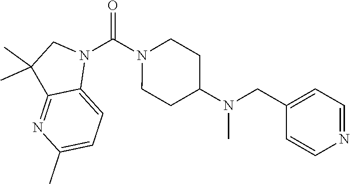 [SMILES: Cc1ccc2c(n1)C(C)(C)CN2C(=O)N1CCC(N(C)Cc2ccncc2)CC1] | (4-(methyl(pyridin-4- ylmethyl)amino)piperidin-1-yl)(3,3,5- trimethyl-2,3-dihydro-1H-pyrrolo[3,2- b]pyridin-1-yl)methanone | 3.98 | 394.2 | A |
| 76 | 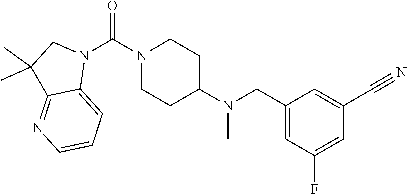 [SMILES: CN(Cc1cc(F)cc(C#N)c1)C1CCN(C(=O)N2CC(C)(C)c3ncccc32)CC1] | 3-(((1-(3,3-dimethyl-2,3-dihydro-1H- pyrrolo[3,2-b]pyridine-1- carbonyl)piperidin-4- yl)(methyl)amino)methyl)-5- fluorobenzonitrile | 4.89 | 422.2 | A |
| 77 | 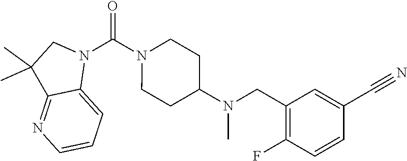 [SMILES: CN(Cc1cc(C#N)ccc1F)C1CCN(C(=O)N2CC(C)(C)c3ncccc32)CC1] | 3-(((1-(3,3-dimethyl-2,3-dihydro-1H- pyrrolo[3,2-b]pyridine-1- carbonyl)piperidin-4- yl)(methyl)amino)methyl)-4- fluorobenzonitrile | 4.67 | 422.2 | A |
| 78 | 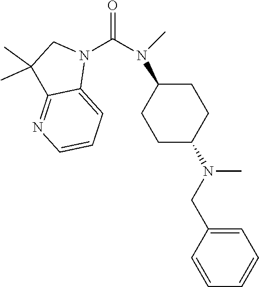 [SMILES: CN(Cc1ccccc1)[C@H]1CC[C@H](N(C)C(=O)N2CC(C)(C)c3ncccc32)CC1] | N-((1r,4r)-4- (benzyl(methyl)amino)cyclohexyl)-N,3,3- trimethyl-2,3-dihydro-1H-pyrrolo[3,2- b]pyridine-1-carboxamide | 4.85 | 407.3 | A |

### Example 79: (4-((3,4-Difluorobenzyl)(methyl)amino)piperidin-1-yl)(3,3-dimethyl-2,3-dihydro-1H-pyrrolo[3,2-b]pyridin-1-yl)methanone

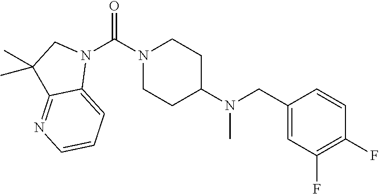 [SMILES: CN(Cc1ccc(F)c(F)c1)C1CCN(C(=O)N2CC(C)(C)c3ncccc32)CC1]

Step 1. tert-Butyl (1-(3,3-dimethyl-2,3-dihydro-1H-pyrrolo[3,2-b]pyridine-1-carbonyl)piperidin-4-yl)(methyl)carbamate: Following the experimental procedure described in Example 1, starting from Intermediate 4B (492 mg, 4.67 mmol) and tert-butyl methyl(piperidin-4-yl)carbamate (1.0 g, 4.67 mmol), the title compound was obtained (1.65 g, 91% yield).

Step 2. (3,3-Di methyl-2,3-dihydro-1H-pyrrolo[3,2-b]pyridin-1-yl)(4-(methylamino)piperidin-1-yl)methanone: To a solution of the product obtained in Step 1 (1.65 g, 4.25 mmol) in 1,4-dioxane (15 mL), HCl solution (4 N in 1,4-dioxane, 10.6 mL, 42.5 mmol) was added and the mixture was stirred at r.t. overnight. The solvent was evaporated and the residue was dissolved in DCM that was washed with 1 N NaOH. The aqueous layer was back-extracted with DCM. The combined organic extracts were dried over MgSO₄, filtered and concentrated to dryness to give the title compound (1.26 g, quant. yield).

Step 3. Title compound: To a solution of the product obtained in Step 2 (143 mg, 0.50 mmol) in THF (4 mL), 3,4-difluorobenzaldehyde (0.08 mL, 0.74 mmol) was added under a N₂ atmosphere and the mixture was stirred at r.t. for 15 min. Then, NaBH(OAc)₃ (315 mg, 1.5 mmol) was added and the reaction mixture was stirred at r.t. overnight. The solvent was evaporated and the residue was dissolved in DCM that was washed with 1 N NaOH. The aqueous layer was back-extracted with DCM. The combined organic extracts were dried over MgSO₄, filtered and concentrated to dryness. The residue was purified by flash chromatography, silica gel, gradient DCM to MeOH:DCM (1:4), to give the title compound (163 mg, 79% yield).

HPLC retention time (Method A): 5.08 min; MS: 415.2 (M+H).

This method was used for the preparation of Examples 80-108 using suitable starting

| | | | Ret | | |
|---|---|---|---|---|---|
| | | | time | MS | |
| EX | Structure | Chemical name | (min) | (M + H) | Method |
| 80 | 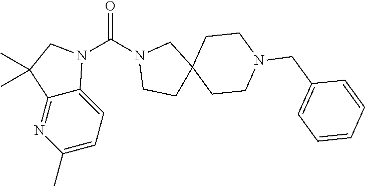 [SMILES: Cc1ccc2c(n1)C(C)(C)CN2C(=O)N1CCC2(CCN(Cc3ccccc3)CC2)C1] | (8-benzyl-2,8-diazaspiro[4.5]decan-2- yl)(3,3,5-trimethyl-2,3-dihydro-1H- pyrrolo[3,2-b]pyridin-1-yl)methanone | 4.93 | 419.3 | A |
| 81 | 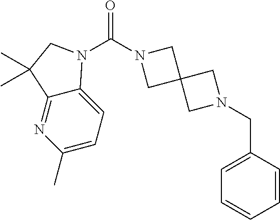 [SMILES: Cc1ccc2c(n1)C(C)(C)CN2C(=O)N1CC2(CN(Cc3ccccc3)C2)C1] | (6-benzyl-2,6-diazaspiro[3.3]heptan-2- yl)(3,3,5-trimethyl-2,3-dihydro-1H- pyrrolo[3,2-b]pyridin-1-yl)methanone | 4.41 | 377.2 | A |
| 82 | 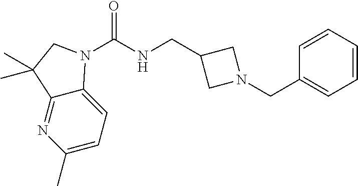 [SMILES: Cc1ccc2c(n1)C(C)(C)CN2C(=O)NCC1CN(Cc2ccccc2)C1] | N-((1-benzylazetidin-3-yl)methyl)-3,3,5- trimethyl-2,3-dihydro-1H-pyrrolo[3,2- b]pyridine-1-carboxamide | 4.18 | 365.2 | A |
| 83 | 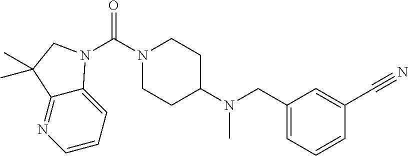 [SMILES: CN(Cc1cccc(C#N)c1)C1CCN(C(=O)N2CC(C)(C)c3ncccc32)CC1] | 3-(((1-(3,3-dimethyl-2,3-dihydro-1H- pyrrolo[3,2-b]pyridine-1-carbonyl)piperidin- 4-yl)(methyl)amino)methyl)benzonitrile | 4.61 | 404.2 | A |
| 84 | 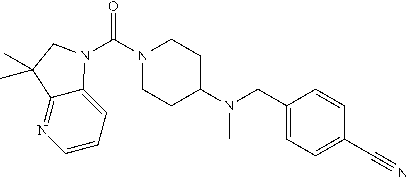 [SMILES: CN(Cc1ccc(C#N)cc1)C1CCN(C(=O)N2CC(C)(C)c3ncccc32)CC1] | 4-(((1-(3,3-dimethyl-2,3-dihydro-1H- pyrrolo[3,2-b]pyridine-1-carbonyl)piperidin- 4-yl)(methyl)amino)methyl)benzonitrile | 4.61 | 404.2 | A |
| 85 | 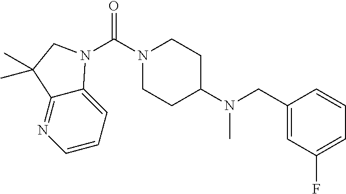 [SMILES: CN(Cc1cccc(F)c1)C1CCN(C(=O)N2CC(C)(C)c3ncccc32)CC1] | (3,3-dimethyl-2,3-dihydro-1H-pyrrolo[3,2- b]pyridin-1-yl)(4-((3- fluorobenzyl)(methyl)amino)piperidin-1- yl)methanone | 4.94 | 397.2 | A |
| 86 | 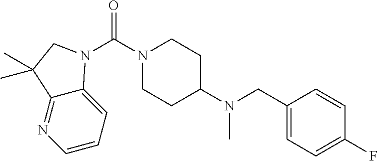 [SMILES: CN(Cc1ccc(F)cc1)C1CCN(C(=O)N2CC(C)(C)c3ncccc32)CC1] | (3,3-dimethyl-2,3-dihydro-1H-pyrrolo[3,2- b]pyridin-1-yl)(4-((4- fluorobenzyl)(methyl)amino)piperidin-1- yl)methanone | 4.83 | 397.2 | A |
| 87 | 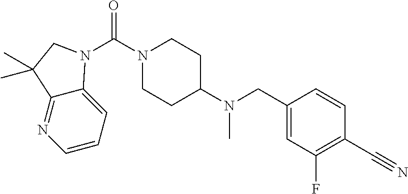 [SMILES: CN(Cc1ccc(C#N)c(F)c1)C1CCN(C(=O)N2CC(C)(C)c3ncccc32)CC1] | 4-(((1-(3,3-dimethyl-2,3-dihydro-1H- pyrrolo[3,2-b]pyridine-1-carbonyl)piperidin- 4-yl)(methyl)amino)methyl)-2- fluorobenzonitrile | 4.86 | 422.2 | A |
| 88 | 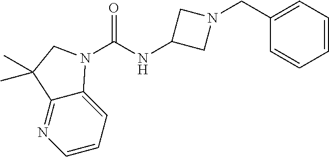 [SMILES: CC1(C)CN(C(=O)NC2CN(Cc3ccccc3)C2)c2cccnc21] | N-(1-benzylazetidin-3-yl)-3,3-dimethyl-2,3- dihydro-1H-pyrrolo[3,2-b]pyridine-1- carboxamide | 4.02 | 337.2 | A |
| 89 | 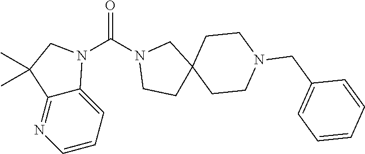 [SMILES: CC1(C)CN(C(=O)N2CCC3(CCN(Cc4ccccc4)CC3)C2)c2cccnc21] | (8-benzyl-2,8-diazaspiro[4.5]decan-2- yl)(3,3-dimethyl-2,3-dihydro-1H-pyrrolo[3,2- b]pyridin-1-yl)methanone | 4.70 | 405.2 | A |
| 90 | 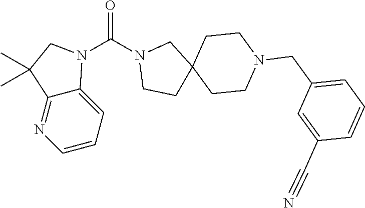 [SMILES: CC1(C)CN(C(=O)N2CCC3(CCN(Cc4cccc(C#N)c4)CC3)C2)c2cccnc21] | 3-((2-(3,3-dimethyl-2,3-dihydro-1H- pyrrolo[3,2-b]pyridine-1-carbonyl)-2,8- diazaspiro[4.5]decan-8- yl)methyl)benzonitrile | 4.60 | 430.2 | A |
| 91 | 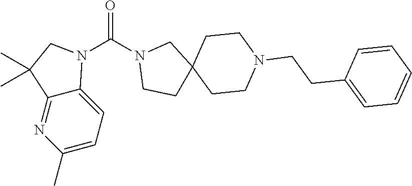 [SMILES: Cc1ccc2c(n1)C(C)(C)CN2C(=O)N1CCC2(CCN(CCc3ccccc3)CC2)C1] | (8-phenethyl-2,8-diazaspiro[4.5]decan-2- yl)(3,3,5-trimethyl-2,3-dihydro-1H- pyrrolo[3,2-b]pyridin-1-yl)methanone | 5.03 | 433 | A |
| 92 | 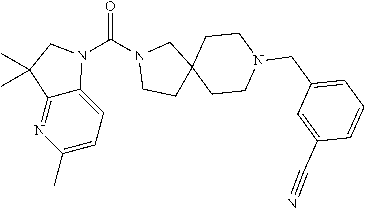 [SMILES: Cc1ccc2c(n1)C(C)(C)CN2C(=O)N1CCC2(CCN(Cc3cccc(C#N)c3)CC2)C1] | 3-((2-(3,3,5-trimethyl-2,3-dihydro-1H- pyrrolo[3,2-b]pyridine-1-carbonyl)-2,8- diazaspiro[4.5]decan-8- yl)methyl)benzonitrile | 4.74 | 444.2 | A |
| 93 | 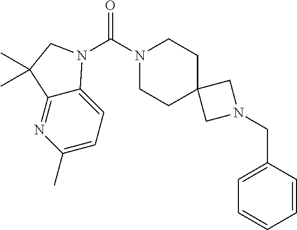 [SMILES: Cc1ccc2c(n1)C(C)(C)CN2C(=O)N1CCC2(CC1)CN(Cc1ccccc1)C2] | (2-benzyl-2,7-diazaspiro[3.5]nonan-7- yl)(3,3,5-trimethyl-2,3-dihydro-1H- pyrrolo[3,2-b]pyridin-1-yl)methanone | 4.64 | 405.2 | A |
| 94 | 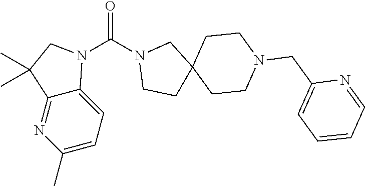 [SMILES: Cc1ccc2c(n1)C(C)(C)CN2C(=O)N1CCC2(CCN(Cc3ccccn3)CC2)C1] | (8-(pyridin-2-ylmethyl)-2,8- diazaspiro[4.5]decan-2-yl)(3,3,5-trimethyl- 2,3-dihydro-1H-pyrrolo[3,2-b]pyridin-1- yl)methanone | 3.96 | 420.1 | A |
| 95 | 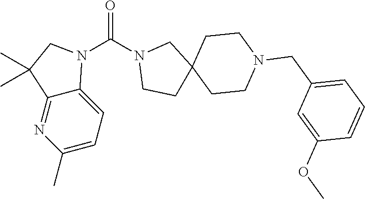 [SMILES: COc1cccc(CN2CCC3(CC2)CCN(C(=O)N2CC(C)(C)c4nc(C)ccc42)C3)c1] | (8-(3-methoxybenzyl)-2,8- diazaspiro[4.5]decan-2-yl)(3,3,5-trimethyl- 2,3-dihydro-1H-pyrrolo[3,2-b]pyridin-1- yl)methanone | 4.88 | 449.2 | A |
| 96 | 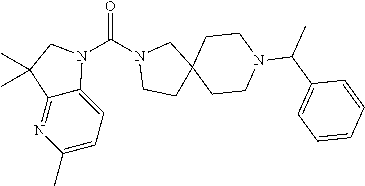 [SMILES: Cc1ccc2c(n1)C(C)(C)CN2C(=O)N1CCC2(CCN(C(C)c3ccccc3)CC2)C1] | (8-(1-phenylethyl)-2,8- diazaspiro[4.5]decan-2-yl)(3,3,5-trimethyl- 2,3-dihydro-1H-pyrrolo[3,2-b]pyridin-1- yl)methanone | 5.00 | 433.2 | A |
| 97 | 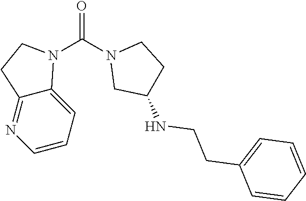 [SMILES: O=C(N1CC[C@H](NCCc2ccccc2)C1)N1CCc2ncccc21] | (S)-(2,3-dihydro-1H-pyrrolo[3,2-b]pyridin-1- yl)(3-(phenethylamino)pyrrolidin-1- yl)methanone | 3.75 | 337.1 | A |
| 98 | 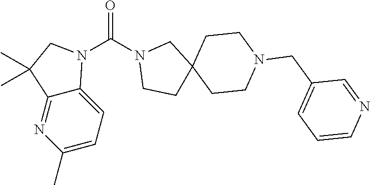 [SMILES: Cc1ccc2c(n1)C(C)(C)CN2C(=O)N1CCC2(CCN(Cc3cccnc3)CC2)C1] | (8-(pyridin-3-ylmethyl)-2,8- diazaspiro[4.5]decan-2-yl)(3,3,5-trimethyl- 2,3-dihydro-1H-pyrrolo[3,2-b]pyridin-1- yl)methanone | 3.92 | 420.2 | A |
| 99 | 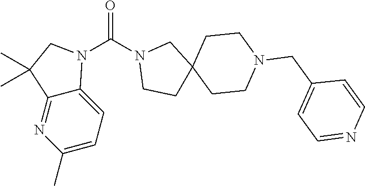 [SMILES: Cc1ccc2c(n1)C(C)(C)CN2C(=O)N1CCC2(CCN(Cc3ccncc3)CC2)C1] | (8-(pyridin-4-ylmethyl)-2,8- diazaspiro[4.5]decan-2-yl)(3,3,5-trimethyl- 2,3-dihydro-1H-pyrrolo[3,2-b]pyridin-1- yl)methanone | 4.00 | 420.2 | A |
| 100 | 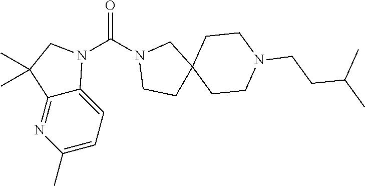 [SMILES: Cc1ccc2c(n1)C(C)(C)CN2C(=O)N1CCC2(CCN(CCC(C)C)CC2)C1] | (8-isopentyl-2,8-diazaspiro[4.5]decan-2- yl)(3,3,5-trimethyl-2,3-dihydro-1H- pyrrolo[3,2-b]pyridin-1-yl)methanone | 4.48 | 399.2 | A |
| 101 | 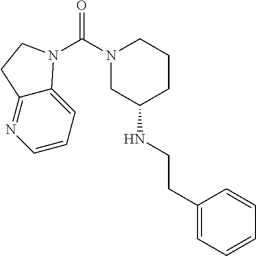 [SMILES: O=C(N1CCC[C@H](NCCc2ccccc2)C1)N1CCc2ncccc21] | (S)-(2,3-dihydro-1H-pyrrolo[3,2-b]pyridin-1- yl)(3-(phenethylamino)piperidin-1- yl)methanone | 3.91 | 351.1 | A |
| 102 | 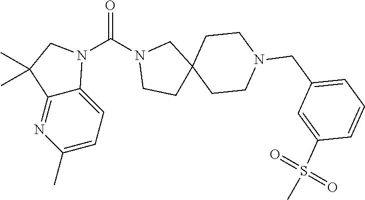 [SMILES: Cc1ccc2c(n1)C(C)(C)CN2C(=O)N1CCC2(CCN(Cc3cccc(S(C)(=O)=O)c3)CC2)C1] | (8-(3-(methylsulfonyl)benzyl)-2,8- diazaspiro[4.5]decan-2-yl)(3,3,5-trimethyl- 2,3-dihydro-1H-pyrrolo[3,2-b]pyridin-1- yl)methanone | 4.29 | 497.1 | A |
| 103 | 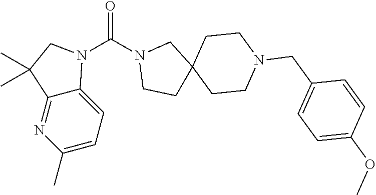 [SMILES: COc1ccc(CN2CCC3(CC2)CCN(C(=O)N2CC(C)(C)c4nc(C)ccc42)C3)cc1] | (8-(4-methoxybenzyl)-2,8- diazaspiro[4.5]decan-2-yl)(3,3,5-trimethyl- 2,3-dihydro-1H-pyrrolo[3,2-b]pyridin-1- yl)methanone | 4.73 | 449.1 | A |
| 104 | 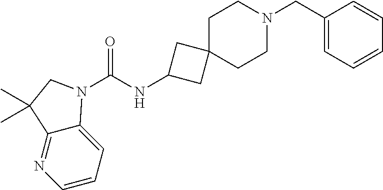 [SMILES: CC1(C)CN(C(=O)NC2CC3(CCN(Cc4ccccc4)CC3)C2)c2cccnc21] | N-(7-benzyl-7-azaspiro[3.5]nonan-2-yl)- 3,3-dimethyl-2,3-dihydro-1H-pyrrolo[3,2- b]pyridine-1-carboxamide | 4.61 | 405.3 | A |
| 105 | 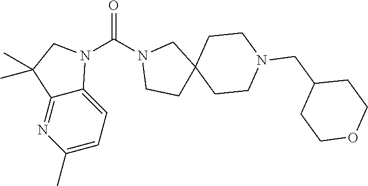 [SMILES: Cc1ccc2c(n1)C(C)(C)CN2C(=O)N1CCC2(CCN(CC3CCOCC3)CC2)C1] | (8-((tetrahydro-2H-pyran-4-yl)methyl)-2,8- diazaspiro[4.5]decan-2-yl)(3,3,5-trimethyl- 2,3-dihydro-1H-pyrrolo[3,2-b]pyridin-1- yl)methanone | 3.92 | 427.3 | A |
| 106 | 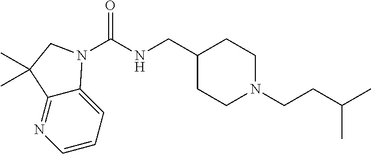 [SMILES: CC(C)CCN1CCC(CNC(=O)N2CC(C)(C)c3ncccc32)CC1] | N-((1-isopentylpiperidin-4-yl)methyl)-3,3- dimethyl-2,3-dihydro-1H-pyrrolo[3,2- b]pyridine-1-carboxamide | 3.74 | 359.3 | A |
| 107 | 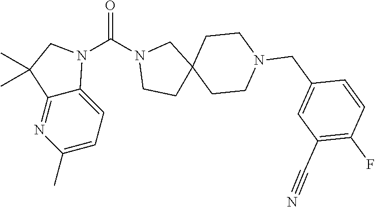 [SMILES: Cc1ccc2c(n1)C(C)(C)CN2C(=O)N1CCC2(CCN(Cc3ccc(F)c(C#N)c3)CC2)C1] | 2-fluoro-5-((2-(3,3,5-trimethyl-2,3-dihydro- 1H-pyrrolo[3,2-b]pyridine-1-carbonyl)-2,8- diazaspiro[4.5]decan-8- yl)methyl)benzonitrile | 4.95 | 462.3 | A |
| 108 | 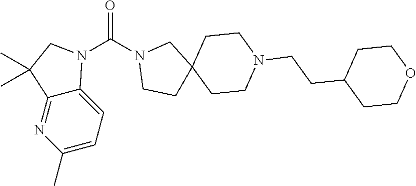 [SMILES: Cc1ccc2c(n1)C(C)(C)CN2C(=O)N1CCC2(CCN(CCC3CCOCC3)CC2)C1] | (8-(2-(tetrahydro-2H-pyran-4-yl)ethyl)-2,8- diazaspiro[4.5]decan-2-yl)(3,3,5-trimethyl- 2,3-dihydro-1H-pyrrolo[3,2-b]pyridin-1- yl)methanone | 3.84 | 441.3 | A |

### Example 109: N-((1-(3,3-Dimethylbutyl)piperidin-4-yl)methyl)-3,3-dimethyl-2,3-dihydro-1H-pyrrolo[3,2-b]pyridine-1-carboxamide

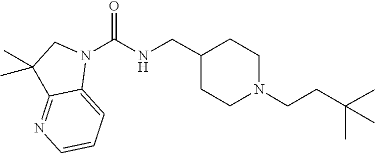 [SMILES: CC(C)(C)CCN1CCC(CNC(=O)N2CC(C)(C)c3ncccc32)CC1]

Step 1. tert-butyl 4-((3,3-dimethyl-2,3-dihydro-1H-pyrrolo[3,2-b]pyridine-1-carboxamido)methyl)piperidine-1-carboxylate: Following the experimental procedure described in Example 1, starting from Intermediate 4B (250 mg, 1.69 mmol) and tert-butyl 4-(aminomethyl)piperidine-1-carboxylate (361 mg, 1.69 mmol), the title compound was obtained (441 mg, 67% yield).

Step 2. 3,3-dimethyl-N-(piperidin-4-ylmethyl)-2,3-dihydro-1H-pyrrolo[3,2-b]pyridine-1-carboxamide: To a solution of the product obtained in Step 1 (441 mg, 1.13 mmol) in DCM (3 mL), TFA (0.44 mL, 5.68 mmol) was added and the mixture was stirred at r.t for 4 h. The solvent was evaporated and the residue was dissolved in DCM that was washed with 1 N aq. NaOH. The aqueous layer was back extracted with DCM. The combined organic extracts were dried over MgSO₄, filtered and concentrated to dryness to give the title compound (327 mg, quant. yield).

Step 3. Title compound: In a sealed tube, a solution of the product obtained in Step 2 (100 mg, 0.35 mmol), K₂CO₃ (96 mg, 0.69 mmol) and 1-bromo-3,3-dimethylbutane (0.05 mL, 0.35 mmol) in ACN (7 mL) was heated at 80° C. for 24 h. Water was added and it was extracted with EtOAc. The combined organic extracts were dried over MgSO₄, filtered and concentrated to dryness. The residue was purified by flash chromatography, silica gel, gradient DCM to MeOH:DCM (1:4), to give the title compound (57 mg, 44% yield).

HPLC retention time (Method A): 4.06 min; MS: 373.3 (M+H).

This method was used for the preparation of Example 110 using suitable starting

| | | | Ret | | |
|---|---|---|---|---|---|
| | | | time | MS | |
| EX | Structure | Chemical name | (min) | (M + H) | Method |
| 110 | 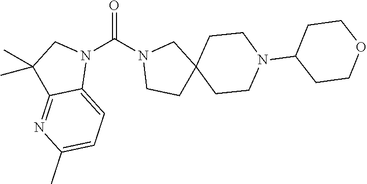 [SMILES: Cc1ccc2c(n1)C(C)(C)CN2C(=O)N1CCC2(CCN(C3CCOCC3)CC2)C1] | (8-(tetrahydro-2H-pyran-4-yl)-2,8- diazaspiro[4.5]decan-2-yl)(3,3,5-trimethyl- 2,3-dihydro-1H-pyrrolo[3,2-b]pyridin-1- yl)methanone | 3.58 | 413.3 | A |

### Example 111: (S)-3,3,5-Trimethyl-N-(2-(methylamino)-2-phenylethyl)-2,3-dihydro-1H-pyrrolo[3,2-b]pyridine-1-carboxamide

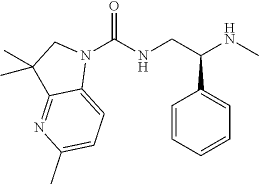

Step 1. (S)-tert-Butyl methyl(1-phenyl-2-(3,3,5-trimethyl-2,3-dihydro-1H-pyrrolo[3,2-b]pyridine-1-carboxamido)ethyl)carbamate: Following the experimental procedure described in Example 1, starting from Intermediate 4D (69 mg, 0.43 mmol) and Intermediate 1E (107 mg, 0.43 mmol), the title compound was obtained (109 mg, 58% yield).

Step 2. Title compound: To a solution of the product obtained in Step 1 (109 mg, 0.25 mmol) in MeOH (2.5 mL), under a N₂ atmosphere, HCl solution (1.25 M in MeOH, 3 mL, 3.75 mmol) was added and the mixture was stirred at r.t. overnight. The solvent was evaporated and the residue was dissolved in DCM that was washed with 1 N aq. NaOH. The aqueous layer was back-extracted with DCM. The combined organic extracts were dried over MgSO₄, filtered and concentrated to dryness. The crude product was purified by flash chromatography, silica gel, gradient DCM to MeOH:DCM (1:4), to give the title compound (24 mg, 23% yield).

HPLC retention time (Method A): 3.96 min; MS: 339.2 (M+H).

This method was used for the preparation of Examples 112-113 using suitable starting

| | | | Ret | | |
|---|---|---|---|---|---|
| | | | time | MS | |
| EX | Structure | Chemical name | (min) | (M + H) | Method |
| 112 | 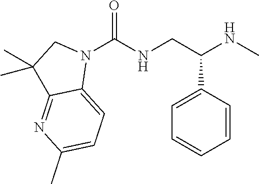 | (R)-3,3,5-trimethyl-N-(2-(methylamino)-2- phenylethyl)-2,3-dihydro-1H-pyrrolo[3,2- b]pyridine-1-carboxamide | 3.97 | 339.2 | A |
| 113 | 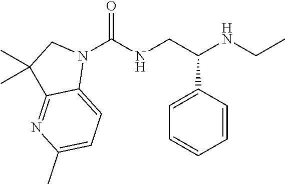 [SMILES: CCN[C@@H](CNC(=O)N1CC(C)(C)c2nc(C)ccc21)c1ccccc1] | (R)-N-(2-(ethylamino)-2-phenylethyl)-3,3,5- trimethyl-2,3-dihydro-1H-pyrrolo[3,2- b]pyridine-1-carboxamide | 4.08 | 353.1 | A |

### Example 114: (4-((4-Fluorobenzyl)(methyl)amino)piperidin-1-yl)(3,3,5-trimethyl-2,3-dihydro-1H-pyrrolo[3,2-b]pyridin-1-yl)methanone

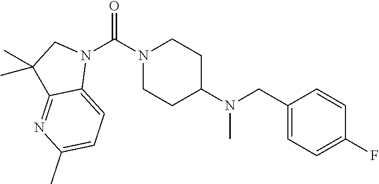 [SMILES: Cc1ccc2c(n1)C(C)(C)CN2C(=O)N1CCC(N(C)Cc2ccc(F)cc2)CC1]

Step 1. 1-(3,3,5-Tri methyl-2,3-dihydro-1H-pyrrolo[3,2-b]pyridine-1-carbonyl)piperidin-4-one: Following the experimental procedure described in Example 1, starting from Intermediate 4D (473 mg, 3.08 mmol) and piperidin-4-one hydrochloride hydrate (500 mg, 3.08 mmol), the title compound was obtained (801 mg, 90% yield).

Step 2. Title compound: Following the experimental procedure described in Step 3 of Example 79, starting from the product obtained in Step 2 (80 mg, 0.28 mmol) and 1-(4-fluorophenyl)-N-methylmethanamine (39 mg, 0.28 mmol), the title compound was obtained (37 mg, 32% yield).

HPLC retention time (Method A): 5.07 min; MS: 411.3 (M+H).

This method was used for the preparation of Examples 115-119 using suitable starting

| | | | Ret | | |
|---|---|---|---|---|---|
| | | | time | MS | |
| EX | Structure | Chemical name | (min) | (M + H) | Method |
| 115 | 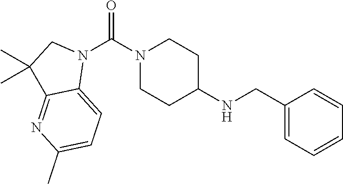 [SMILES: Cc1ccc2c(n1)C(C)(C)CN2C(=O)N1CCC(NCc2ccccc2)CC1] | (4-(benzylamino)piperidin-1-yl)(3,3,5- trimethyl-2,3-dihydro-1H-pyrrolo[3,2- b]pyridin-1-yl)methanone | 4.39 | 379.2 | A |
| 116 | 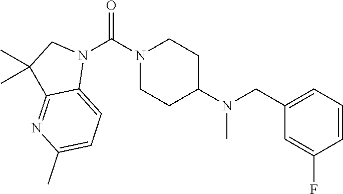 [SMILES: Cc1ccc2c(n1)C(C)(C)CN2C(=O)N1CCC(N(C)Cc2cccc(F)c2)CC1] | (4-((3- fluorobenzyl)(methyl)amino)piperidin-1- yl)(3,3,5-trimethyl-2,3-dihydro-1H- pyrrolo[3,2-b]pyridin-1-yl)methanone | 5.16 | 411.2 | A |
| 117 | 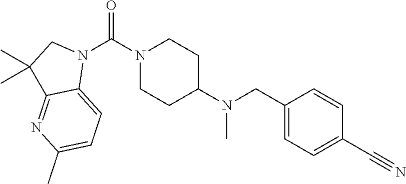 [SMILES: Cc1ccc2c(n1)C(C)(C)CN2C(=O)N1CCC(N(C)Cc2ccc(C#N)cc2)CC1] | 4-((methyl(1-(3,3,5-trimethyl-2,3-dihydro- 1H-pyrrolo[3,2-b]pyridine-1- carbonyl)piperidin-4- yl)amino)methyl)benzonitrile | 4.83 | 418.2 | A |
| 118 | 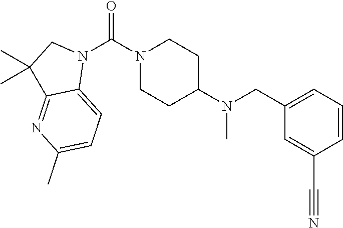 [SMILES: Cc1ccc2c(n1)C(C)(C)CN2C(=O)N1CCC(N(C)Cc2cccc(C#N)c2)CC1] | 3-((methyl(1-(3,3,5-trimethyl-2,3-dihydro- 1H-pyrrolo[3,2-b]pyridine-1- carbonyl)piperidin-4- yl)amino)methyl)benzonitrile | 4.83 | 418.2 | A |
| 119 | 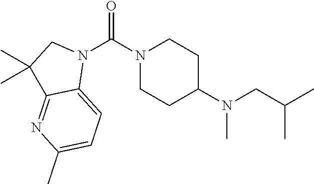 [SMILES: Cc1ccc2c(n1)C(C)(C)CN2C(=O)N1CCC(N(C)CC(C)C)CC1] | (4-(isobutyl(methyl)amino)piperidin-1- yl)(3,3,5-trimethyl-2,3-dihydro-1H- pyrrolo[3,2-b]pyridin-1-yl)methanone | 4.51 | 359.2 | A |

### Example 120: (3-(Isopentylamino)azepan-1-yl)(3,3,5-trimethyl-2,3-dihydro-1H-pyrrolo[3,2-b]pyridin-1-yl)methanone

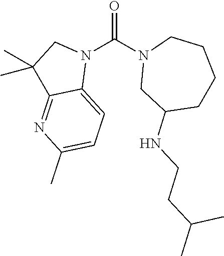 [SMILES: Cc1ccc2c(n1)C(C)(C)CN2C(=O)N1CCCCC(NCCC(C)C)C1]

Step 1. (3-(Benzyl(isopentyl)amino)azepan-1-yl)(3,3,5-trimethyl-2,3-dihydro-1H-pyrrolo[3,2-b]pyridin-1-yl)methanone: Following the experimental procedure described in Example 1, starting from Intermediate 4D (59 mg, 0.36 mmol) and Intermediate 2G (100 mg, 0.36 mmol), the title compound was obtained (93 mg, 55% yield).

Step 2. Title compound: A solution of the product obtained in Step 1 (93 mg, 0.20 mmol) in EtOAc (5 mL) was purged with N₂ in a pressure tube. Palladium (10 mg, 10% wt. on charcoal, wet) was added. The tube was purged with H₂ and the reaction mixture was stirred at r.t. under 2 bars of H₂ overnight. The catalyst was filtered off and the solvent was evaporated. The residue was submitted to a second reaction cycle with fresh catalyst, this time heating at 50° C. under 2 bars of H₂ overnight to get full conversion. The catalyst was filtered off and the solvent was evaporated. The crude product was purified by flash chromatography, silica gel, gradient DCM to MeOH:DCM (1:4), to give the title compound (35 mg, 46% yield).

HPLC retention time (Method A): 5.26 min; MS: 373.1 (M+H).

This method was used for the preparation of Example 121 using suitable starting

| | | | Ret | | |
|---|---|---|---|---|---|
| | | | time | MS | |
| EX | Structure | Chemical name | (min) | (M + H) | Method |
| 121 | 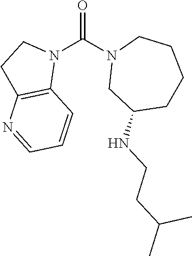 [SMILES: CC(C)CCN[C@H]1CCCCN(C(=O)N2CCc3ncccc32)C1] | (S)-(2,3-dihydro-1H-pyrrolo[3,2-b]pyridin-1- yl)(3-(isopentylamino)azepan-1- yl)methanone | 3.64 | 331.1 | A |

### Example 122: (S)-(2,3-Dihydro-1H-pyrrolo[3,2-b]pyridin-1-yl)(3-(isopentyl(methyl)amino)azepan-1-yl)methanone

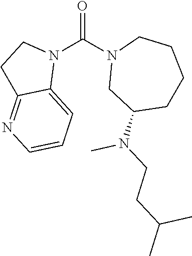 [SMILES: CC(C)CCN(C)[C@H]1CCCCN(C(=O)N2CCc3ncccc32)C1]

To a solution of Example 121 (47 mg, 0.142 mmol) in MeOH (1 mL), formaldehyde (0.13 mL, 1.42 mmol) and acetic acid (0.02 mL, 0.36 mmol) were added and the reaction mixture was stirred at r.t for 30 min. Then, NaBH(OAc)₃ (75 mg, 0.36 mmol) was added and the mixture was stirred at r.t. overnight. Aq. NaHCO₃ sat. solution was added and it was extracted with DCM. The combined organic extracts were washed with brine, dried over MgSO₄, filtered and concentrated to dryness. The residue was purified by flash chromatography, silica gel, gradient DCM to MeOH:DCM (1:4), to give the title compound (10 mg, 20% yield).

HPLC retention time (Method A): 4.17 min; MS: 345.1 (M+H).

### Example 123: (1-Benzylpiperidin-4-yl)(3,3,5-trimethyl-2,3-dihydro-1H-pyrrolo[3,2-b]pyridin-1-yl)methanone

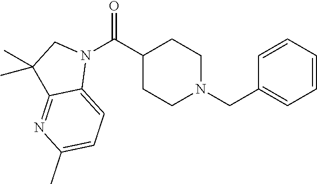 [SMILES: Cc1ccc2c(n1)C(C)(C)CN2C(=O)C1CCN(Cc2ccccc2)CC1]

To a solution of 1-benzylpiperidine-4-carboxylic acid (50 mg, 0.23 mmol) and Intermediate 4D (37 mg, 0.23 mmol) in DMF (2.3 mL), DIPEA (0.12 mL, 0.68 mmol) and HATU (87 mg, 0.23 mmol) were added and the reaction mixture was stirred at r.t. overnight. Aq. NaHCO₃ sat. solution was added and it was extracted with EtOAc. The combined organic extracts were washed with water and brine, dried over MgSO₄, filtered and concentrated to dryness. The crude product was purified by flash chromatography, silica gel, gradient DCM to MeOH:DCM (1:4), to give the title compound (41 mg, 49% yield).

HPLC retention time (Method A): 4.88 min; MS: 364.2 (M+H).

This method was used for the preparation of Examples 124-127 using suitable starting

| | | | Ret | | |
|---|---|---|---|---|---|
| | | | time | MS | |
| EX | Structure | Chemical name | (min) | (M + H) | Method |
| 124 | 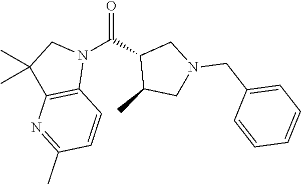 [SMILES: Cc1ccc2c(n1)C(C)(C)CN2C(=O)[C@@H]1CN(Cc2ccccc2)C[C@H]1C] | ((3S,4S)-1-benzyl-4-methylpyrrolidin-3- yl)(3,3,5-trimethyl-2,3-dihydro-1H- pyrrolo[3,2-b]pyridin-1-yl)methanone | 5.13 | 364.2 | A |
| 125 | 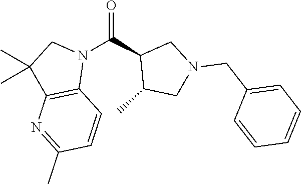 [SMILES: Cc1ccc2c(n1)C(C)(C)CN2C(=O)[C@H]1CN(Cc2ccccc2)C[C@@H]1C] | ((3R,4R)-1-benzyl-4-methylpyrrolidin-3- yl)(3,3,5-trimethyl-2,3-dihydro-1H- pyrrolo[3,2-b]pyridin-1-yl)methanone | 5.14 | 364.2 | A |
| 126 | 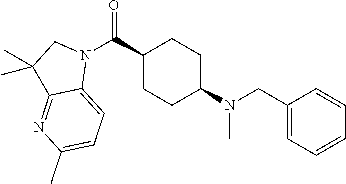 [SMILES: Cc1ccc2c(n1)C(C)(C)CN2C(=O)[C@H]1CC[C@@H](N(C)Cc2ccccc2)CC1] | ((1s,4s)-4- (benzyl(methyl)amino)cyclohexyl)(3,3,5- trimethyl-2,3-dihydro-1H-pyrrolo[3,2- b]pyridin-1-yl)methanone | 5.58 | 392.2 | A |
| 127 | 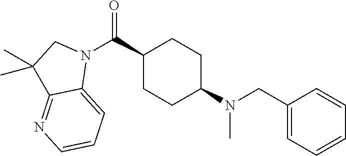 [SMILES: CN(Cc1ccccc1)[C@H]1CC[C@@H](C(=O)N2CC(C)(C)c3ncccc32)CC1] | ((1s,4s)-4- (benzyl(methyl)amino)cyclohexyl)(3,3- dimethyl-2,3-dihydro-1H-pyrrolo[3,2- b]pyridin-1-yl)methanone | 5.31 | 378.2 | A |

### Example 128: ((1r,4r)-4-(Benzylamino)cyclohexyl)(3,3-dimethyl-2,3-dihydro-1H-pyrrolo[3,2-b]pyridin-1-yl)methanone dihydrochloride

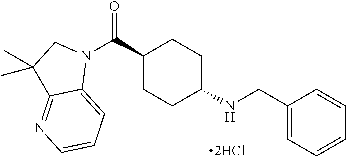 [SMILES: CC1(C)CN(C(=O)[C@H]2CC[C@H](NCc3ccccc3)CC2)c2cccnc21]

Step 1. tert-Butyl benzyl((1r,4r)-4-(3,3-dimethyl-2,3-dihydro-1H-pyrrolo[3,2-b]pyridine-1-carbonyl)cyclohexyl)carbamate: Following the experimental procedure described in Example 123, starting from Intermediate 4B (100 mg, 0.67 mmol) and (1r,4r)-4-(benzyl(tert-butoxycarbonyl)amino)cyclohexane-1-carboxylic acid (224 mg, 0.67 mmol), the title compound was obtained (39 mg, 12% yield).

Step 2. Title compound: To a solution of the product obtained in Step 1 (39 mg, 0.084 mmol) in MeOH (2 mL), HCl solution (4 M in 1,4-dioxane, 0.21 mL, 0.84 mmol) was added. The reaction mixture was stirred at r.t. overnight. The solvent was concentrated in vacuo to afford the title compound (36 mg, 98% yield).

HPLC retention time (Method A): 4.15 min; MS: 364.1 (M+H).

This method was used for the preparation of Example 129 using suitable starting materials:

| | | | Ret | | |
|---|---|---|---|---|---|
| | | | time | MS | |
| EX | Structure | Chemical name | (min) | (M + H) | Method |
| 129 | 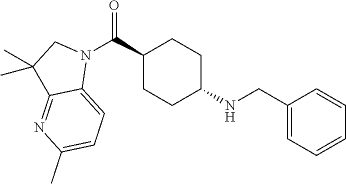 [SMILES: Cc1ccc2c(n1)C(C)(C)CN2C(=O)[C@H]1CC[C@H](NCc2ccccc2)CC1] | ((1r,4r)-4-(benzylamino)cyclohexyl)(3,3,5- trimethyl-2,3-dihydro-1H-pyrrolo[3,2- b]pyridin-1-yl)methanone dihydrochloride | 4.39 | 378.1 | A |

### Example 130: 2-(1-Benzylpiperidin-4-yl)-1-(3,3,5-trimethyl-2,3-dihydro-1H-pyrrolo[3,2-b]pyridin-1-yl)ethanone

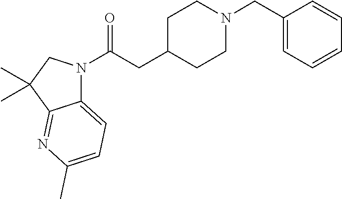 [SMILES: Cc1ccc2c(n1)C(C)(C)CN2C(=O)CC1CCN(Cc2ccccc2)CC1]

Step 1. tert-Butyl 4-(2-oxo-2-(3,3,5-trimethyl-2,3-dihydro-1H-pyrrolo[3,2-b]pyridin-1-yl)ethyl)piperidine-1-carboxylate: Following the experimental procedure described in Example 123, starting from Intermediate 4D (100 mg, 0.62 mmol) and 2-(1-(tert-butoxycarbonyl)piperidin-4-yl)acetic acid (150 mg, 0.62 mmol), the title compound was obtained (214 mg, 90% yield).

Step 2. 2-(Piperidin-4-yl)-1-(3,3,5-trimethyl-2,3-dihydro-1H-pyrrolo[3,2-b]pyridin-1-yl)ethan-1-one: To a solution of the product obtained in Step 1 (100 mg, 0.26 mmol) in DCM (4 mL), TFA (0.2 mL, 2.60 mmol) was added and the resulting mixture was stirred at r.t. overnight. The solvent was evaporated and the residue was partitioned between DCM and 1 N aq. NaOH. The aqueous layer was back-extracted with DCM. The combined organic extracts were washed with brine, dried over MgSO₄, filtered and concentrated to dryness to afford the title compound (60 mg, 81% yield).

Step 3. Title compound: Following the experimental procedure described in Step 3 of Example 79, starting from the product obtained in Step 2 (60 mg, 0.21 mmol) and benzaldehyde (0.03 mL, 0.31 mmol), the title compound was obtained (46 mg, 58% yield).

HPLC retention time (Method A): 4.99 min; MS: 378.2 (M+H).

This method was used for the preparation of Examples 131-136 using suitable starting

| | | | Ret | | |
|---|---|---|---|---|---|
| | | | time | MS | |
| EX | Structure | Chemical name | (min) | (M + H) | Method |
| 131 | 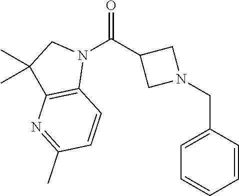 [SMILES: Cc1ccc2c(n1)C(C)(C)CN2C(=O)C1CN(Cc2ccccc2)C1] | (1-benzylazetidin-3-yl)(3,3,5-trimethyl-2,3- dihydro-1H-pyrrolo[3,2-b]pyridin-1- yl)methanone | 4.47 | 336.2 | A |
| 132 | 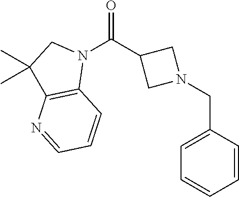 [SMILES: CC1(C)CN(C(=O)C2CN(Cc3ccccc3)C2)c2cccnc21] | (1-benzylazetidin-3-yl)(3,3-dimethyl-2,3- dihydro-1H-pyrrolo[3,2-b]pyridin-1- yl)methanone | 4.26 | 322.1 | A |
| 133 | 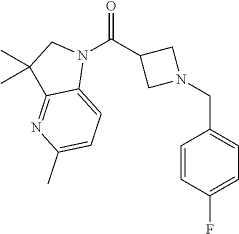 [SMILES: Cc1ccc2c(n1)C(C)(C)CN2C(=O)C1CN(Cc2ccc(F)cc2)C1] | (1-(4-fluorobenzyl)azetidin-3-yl)(3,3,5- trimethyl-2,3-dihydro-1H-pyrrolo[3,2- b]pyridin-1-yl)methanone | 4.54 | 354.1 | A |
| 134 | 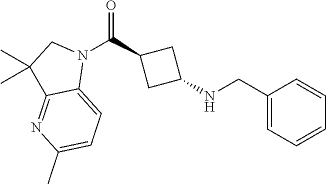 [SMILES: Cc1ccc2c(n1)C(C)(C)CN2C(=O)[C@H]1C[C@H](NCc2ccccc2)C1] | ((1r,3r)-3-(benzylamino)cyclobutyl)(3,3,5- trimethyl-2,3-dihydro-1H-pyrrolo[3,2- b]pyridin-1-yl)methanone | 4.41 | 350.1 | A |
| 135 | 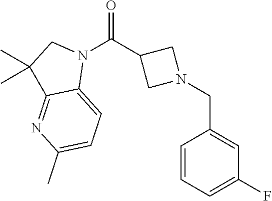 [SMILES: Cc1ccc2c(n1)C(C)(C)CN2C(=O)C1CN(Cc2cccc(F)c2)C1] | (1-(3-fluorobenzyl)azetidin-3-yl)(3,3,5- trimethyl-2,3-dihydro-1H-pyrrolo[3,2- b]pyridin-1-yl)methanone | 4.62 | 354.1 | A |
| 136 | 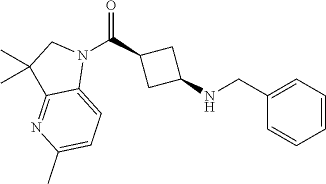 [SMILES: Cc1ccc2c(n1)C(C)(C)CN2C(=O)[C@H]1C[C@@H](NCc2ccccc2)C1] | ((1s,3s)-3-(benzylamino)cyclobutyl)(3,3,5- trimethyl-2,3-dihydro-1H-pyrrolo[3,2- b]pyridin-1-yl)methanone | 4.40 | 350.1 | A |

### Example 137: ((1r,3r)-3-(Benzyl(methyl)amino)cyclobutyl)(3,3,5-trimethyl-2,3-dihydro-1H-pyrrolo[3,2-b]pyridin-1-yl)methanone

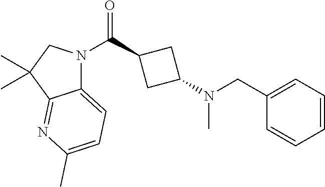 [SMILES: Cc1ccc2c(n1)C(C)(C)CN2C(=O)[C@H]1C[C@H](N(C)Cc2ccccc2)C1]

Starting from Example 134 (76 mg, 0.22 mmol) and following the experimental procedure described for the preparation of Example 122, the title compound was obtained (49 mg, 62% yield).

HPLC retention time (Method A): 5.03 min; MS: 364.1 (M+H).

This method was used for the preparation of Examples 138-140 using the corresponding examples as starting materials:

| | | | Ret | | |
|---|---|---|---|---|---|
| | | | time | MS | |
| EX | Structure | Chemical name | (min) | (M + H) | Method |
| 138 | 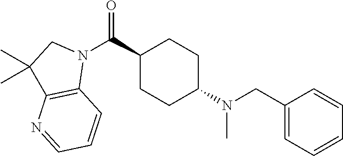 [SMILES: CN(Cc1ccccc1)[C@H]1CC[C@H](C(=O)N2CC(C)(C)c3ncccc32)CC1] | ((1r,4r)-4- (benzyl(methyl)amino)cyclohexyl)(3,3- dimethyl-2,3-dihydro-1H-pyrrolo[3,2- b]pyridin-1-yl)methanone | 4.89 | 378.1 | A |
| 139 | 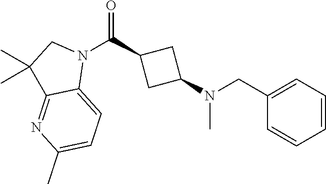 [SMILES: Cc1ccc2c(n1)C(C)(C)CN2C(=O)[C@H]1C[C@@H](N(C)Cc2ccccc2)C1] | ((1s,3s)-3- (benzyl(methyl)amino)cyclobutyl)(3,3,5- trimethyl-2,3-dihydro-1H-pyrrolo[3,2- b]pyridin-1-yl)methanone | 5.04 | 364.1 | A |
| 140 | 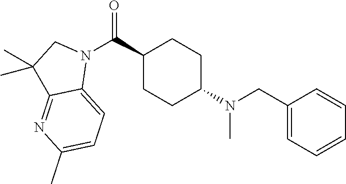 [SMILES: Cc1ccc2c(n1)C(C)(C)CN2C(=O)[C@H]1CC[C@H](N(C)Cc2ccccc2)CC1] | ((1r,4r)-4- (benzyl(methyl)amino)cyclohexyl)(3,3,5- trimethyl-2,3-dihydro-1H-pyrrolo[3,2- b]pyridin-1-yl)methanone (1) | 5.13 | 392.2 | A |

### Examples 141-158

The following examples were synthesized following the method described in Example 1 using suitable starting materials:

| | | | Ret | | |
|---|---|---|---|---|---|
| | | | time | MS | |
| EX | Structure | Chemical name | (min) | (M + H) | Method |
| 141 | 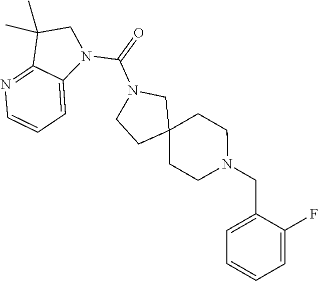 [SMILES: CC1(C)CN(C(=O)N2CCC3(CCN(Cc4ccccc4F)CC3)C2)c2cccnc21] | (3,3-dimethyl-2,3-dihydro-1H-pyrrolo[3,2- b]pyridin-1-yl)-(8-(2-fluorobenzyl)-2,8- diazaspiro[4.5]decan-2-yl)methanone | 2.01 | 423.3 | C |
| 142 | 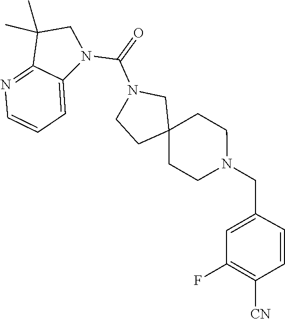 [SMILES: CC1(C)CN(C(=O)N2CCC3(CCN(Cc4ccc(C#N)c(F)c4)CC3)C2)c2cccnc21] | 4-((2-(3,3-dimethyl-2,3-dihydro-1H- pyrrolo[3,2-b]pyridine-1-carbonyl)-2,8- diazaspiro[4.5]decan-8-yl)methyl)-2- fluorobenzonitrile | 2.00 | 448.2 | C |
| 143 | 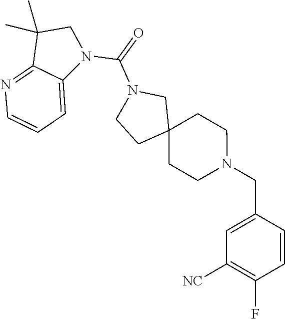 [SMILES: CC1(C)CN(C(=O)N2CCC3(CCN(Cc4ccc(F)c(C#N)c4)CC3)C2)c2cccnc21] | 5-((2-(3,3-dimethyl-2,3-dihydro-1H- pyrrolo[3,2-b]pyridine-1-carbonyl)-2,8- diazaspiro[4.5]decan-8-yl)methyl)-2- fluorobenzonitrile | 1.96 | 448.2 | C |
| 144 | 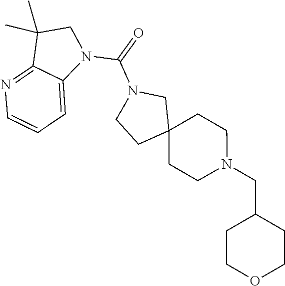 [SMILES: CC1(C)CN(C(=O)N2CCC3(CCN(CC4CCOCC4)CC3)C2)c2cccnc21] | (3,3-dimethyl-2,3-dihydro-1H-pyrrolo[3,2- b]pyridin-1-yl)(8-((tetrahydro-2H-pyran-4- yl)methyl)-2,8-diazaspiro[4.5]decan-2- yl)methanone | 1.45 | 413.3 | C |
| 145 | 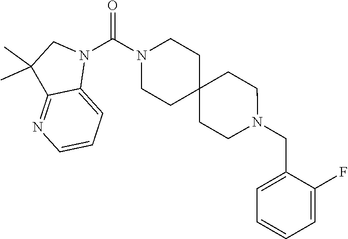 [SMILES: CC1(C)CN(C(=O)N2CCC3(CCN(Cc4ccccc4F)CC3)CC2)c2cccnc21] | (3,3-dimethyl-2,3-dihydro-1H-pyrrolo[3,2- b]pyridin-1-yl)(9-(2-fluorobenzyl)-3,9- diazaspiro[5.5]undecan-3-yl)methanone | 2.23 | 437.2 | B |
| 146 | 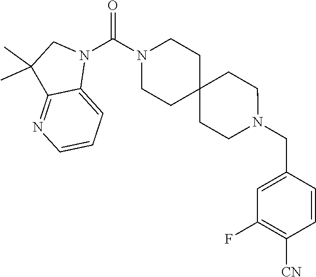 [SMILES: CC1(C)CN(C(=O)N2CCC3(CCN(Cc4ccc(C#N)c(F)c4)CC3)CC2)c2cccnc21] | 4-((9-(3,3-dimethyl-2,3-dihydro-1H- pyrrolo[3,2-b]pyridine-1-carbonyl)-3,9- diazaspiro[5.5]undecan-3-yl)methyl)-2- fluorobenzonitrile | 2.22 | 462.2 | B |
| 147 | 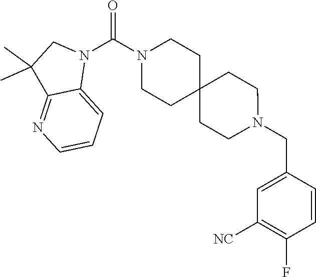 [SMILES: CC1(C)CN(C(=O)N2CCC3(CCN(Cc4ccc(F)c(C#N)c4)CC3)CC2)c2cccnc21] | 5-((9-(3,3-dimethyl-2,3-dihydro-1H- pyrrolo[3,2-b]pyridine-1-carbonyl)-3,9- diazaspiro[5.5]undecan-3-yl)methyl)-2- fluorobenzonitrile | 2.18 | 462.2 | B |
| 148 | 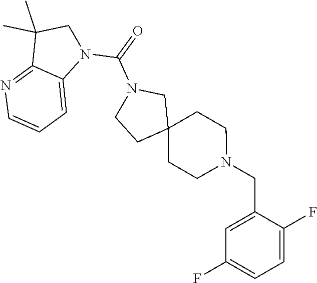 [SMILES: CC1(C)CN(C(=O)N2CCC3(CCN(Cc4cc(F)ccc4F)CC3)C2)c2cccnc21] | (8-(2,5-difluorobenzyl)-2,8- diazaspiro[4.5]decan-2-yl)(3,3-dimethyl-2,3- dihydro-1H-pyrrolo[3,2-b]pyridin-1- yl)methanone | 2.19 | 441.2 | B |
| 149 | 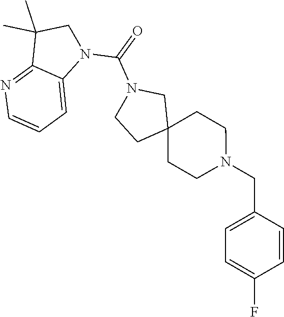 [SMILES: CC1(C)CN(C(=O)N2CCC3(CCN(Cc4ccc(F)cc4)CC3)C2)c2cccnc21] | (3,3-dimethyl-2,3-dihydro-1H-pyrrolo[3,2- b]pyridin-1-yl)(8-(4-fluorobenzyl)-2,8- diazaspiro[4.5]decan-2-yl)methanone | 2.08 | 423.2 | B |
| 150 | 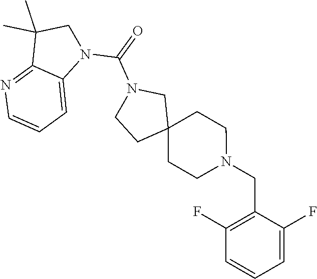 [SMILES: CC1(C)CN(C(=O)N2CCC3(CCN(Cc4c(F)cccc4F)CC3)C2)c2cccnc21] | (8-(2,6-difluorobenzyl)-2,8- diazaspiro[4.5]decan-2-yl)(3,3-dimethyl-2,3- dihydro-1H-pyrrolo[3,2-b]pyridin-1- yl)methanone | 2.11 | 441.2 | B |
| 151 | 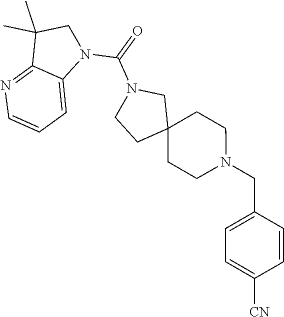 [SMILES: CC1(C)CN(C(=O)N2CCC3(CCN(Cc4ccc(C#N)cc4)CC3)C2)c2cccnc21] | 4-((2-(3,3-dimethyl-2,3-dihydro-1H- pyrrolo[3,2-b]pyridine-1-carbonyl)-2,8- diazaspiro[4.5]decan-8-yl)methyl)benzonitrile | 2.01 | 430.2 | B |
| 152 | 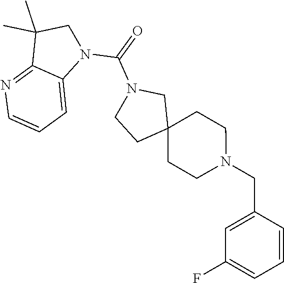 [SMILES: CC1(C)CN(C(=O)N2CCC3(CCN(Cc4cccc(F)c4)CC3)C2)c2cccnc21] | (3,3-dimethyl-2,3-dihydro-1H-pyrrolo[3,2- b]pyridin-1-yl)(8-(3-fluorobenzyl)-2,8- diazaspiro[4.5]decan-2-yl)methanone | 2.18 | 423.2 | B |
| 153 | 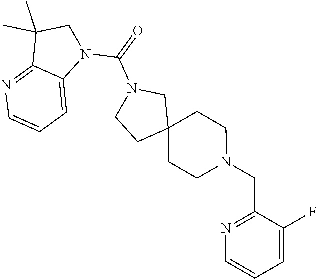 [SMILES: CC1(C)CN(C(=O)N2CCC3(CCN(Cc4ncccc4F)CC3)C2)c2cccnc21] | (3,3-dimethyl-2,3-dihydro-1H-pyrrolo[3,2- b]pyridin-1-yl)(8-((3-fluoropyridin-2- yl)methyl)-2,8-diazaspiro[4.5]decan-2- yl)methanone | 1.66 | 424.2 | B |
| 154 | 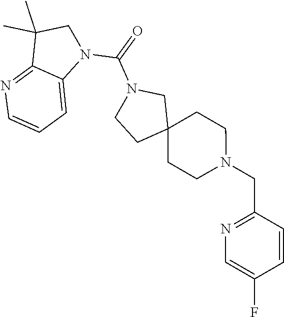 [SMILES: CC1(C)CN(C(=O)N2CCC3(CCN(Cc4ccc(F)cn4)CC3)C2)c2cccnc21] | (3,3-dimethyl-2,3-dihydro-1H-pyrrolo[3,2- b]pyridin-1-yl)(8-((5-fluoropyridin-2- yl)methyl)-2,8-diazaspiro[4.5]decan-2- yl)methanone | 1.74 | 424.2 | B |
| 155 | 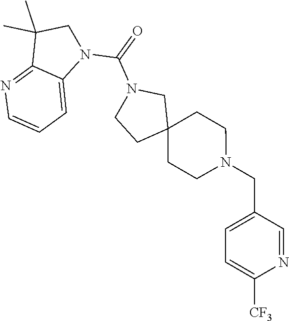 [SMILES: CC1(C)CN(C(=O)N2CCC3(CCN(Cc4ccc(C(F)(F)F)nc4)CC3)C2)c2cccnc21] | (3,3-dimethyl-2,3-dihydro-1H-pyrrolo[3,2- b]pyridin-1-yl)(8-((6-(trifluoromethyl)pyridin-3- yl)methyl)-2,8-diazaspiro[4.5]decan-2- yl)methanone | 2.04 | 474.2 | B |
| 156 | 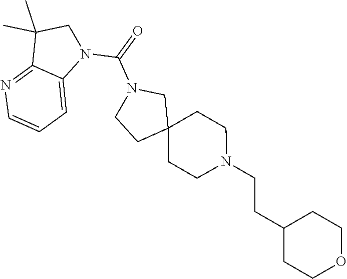 [SMILES: CC1(C)CN(C(=O)N2CCC3(CCN(CCC4CCOCC4)CC3)C2)c2cccnc21] | (3,3-dimethyl-2,3-dihydro-1H-pyrrolo[3,2- b]pyridin-1-yl)(8-(2-(tetrahydro-2H-pyran-4- yl)ethyl)-2,8-diazaspiro[4.5]decan-2- yl)methanone | 1.52 | 427.2 | B |
| 157 | 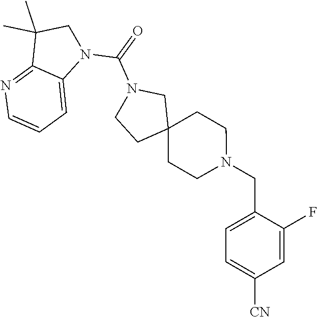 [SMILES: CC1(C)CN(C(=O)N2CCC3(CCN(Cc4ccc(C#N)cc4F)CC3)C2)c2cccnc21] | 4-((2-(3,3-dimethyl-2,3-dihydro-1H- pyrrolo[3,2-b]pyridine-1-carbonyl)-2,8- diazaspiro[4.5]decan-8-yl)methyl)-3- fluorobenzonitrile | 2.00 | 448.2 | B |
| 158 | 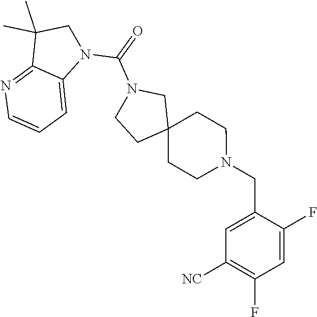 [SMILES: CC1(C)CN(C(=O)N2CCC3(CCN(Cc4cc(C#N)c(F)cc4F)CC3)C2)c2cccnc21] | 5.-((2-(3,3-dimethyl-2,3-dihydro-1H- pyrrolo[3,2-b]pyridine-1-carbonyl)-2,8- diazaspiro[4.5]decan-8-yl)methyl)-2,4- difluorobenzonitrile | 2.02 | 466.0 | C |

### Examples 159-167

The following examples were synthesized following the method described in Example 79 using suitable starting materials:

| | | | Ret | | |
|---|---|---|---|---|---|
| | | | time | MS | |
| EX | Structure | Chemical name | (min) | (M + H) | Method |
| 159 | 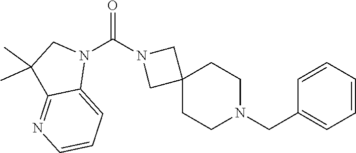 [SMILES: CC1(C)CN(C(=O)N2CC3(CCN(Cc4ccccc4)CC3)C2)c2cccnc21] | (7-benzyl-2,7-diazaspiro[3.5]nonan-2-yl)(3,3- dimethyl-2,3-dihydro-1H-pyrrolo[3,2- b]pyridin-1-yl)methanone | 4.60 | 391.2 | A |
| 160 | 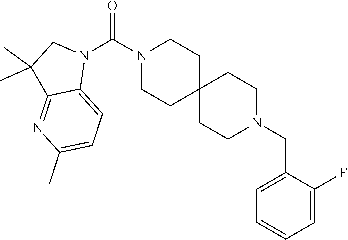 [SMILES: Cc1ccc2c(n1)C(C)(C)CN2C(=O)N1CCC2(CCN(Cc3ccccc3F)CC2)CC1] | (9-(2-fluorobenzyl)-3,9- diazaspiro[5.5]undecan-3-yl)(3,3,5-trimethyl- 2,3-dihydro-1H-pyrrolo[3,2-b]pyridin-1- yl)methanone | 2.33 | 451.2 | B |
| 161 | 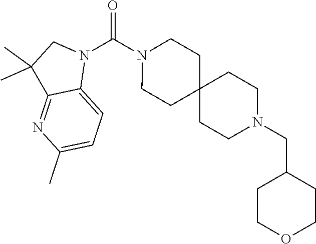 [SMILES: Cc1ccc2c(n1)C(C)(C)CN2C(=O)N1CCC2(CCN(CC3CCOCC3)CC2)CC1] | (9-((tetrahydro-2H-pyran-4-yl)methyl)-3,9- diazaspiro[5.5]undecan-3-yl)(3,3,5-trimethyl- 2,3-dihydro-1H-pyrrolo[3,2-b]pyridin-1- yl)methanone | 1.65 | 441.2 | B |
| 162 | 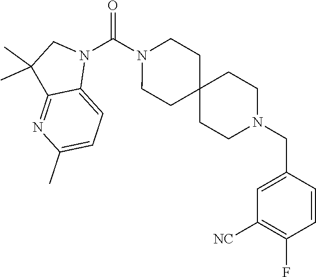 [SMILES: Cc1ccc2c(n1)C(C)(C)CN2C(=O)N1CCC2(CCN(Cc3ccc(F)c(C#N)c3)CC2)CC1] | 2-fluoro-5-((9-(3,3,5-trimethyl-2,3-dihydro- 1H-pyrrolo[3,2-b]pyridine-1-carbonyl)-3,9- diazaspiro[5.5]undecan-3- yl)methyl)benzonitrile | 2.26 | 476.2 | B |
| 163 | 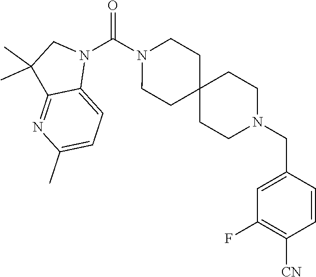 [SMILES: Cc1ccc2c(n1)C(C)(C)CN2C(=O)N1CCC2(CCN(Cc3ccc(C#N)c(F)c3)CC2)CC1] | 2-fluoro-4-((9-(3,3,5-trimethyl-2,3-dihydro- 1H-pyrrolo[3,2-b]pyridine-1-carbonyl)-3,9- diazaspiro[5.5]undecan-3- yl)methyl)benzonitrile | 2.32 | 476.2 | B |
| 164 | 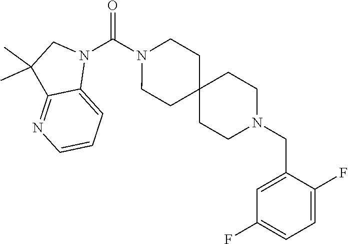 [SMILES: CC1(C)CN(C(=O)N2CCC3(CCN(Cc4cc(F)ccc4F)CC3)CC2)c2cccnc21] | (9-(2,5-difluorobenzyl)-3,9- diazaspiro[5.5]undecan-3-yl)(3,3-dimethyl- 2,3-dihydro-1H-pyrrolo[3,2-b]pyridin-1- yl)methanone | 2.31 | 455.2 | B |
| 165 | 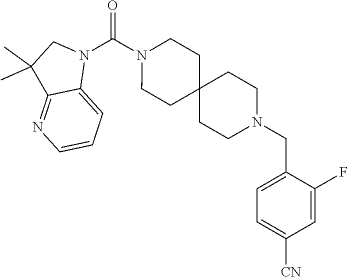 [SMILES: CC1(C)CN(C(=O)N2CCC3(CCN(Cc4ccc(C#N)cc4F)CC3)CC2)c2cccnc21] | 4-((9-(3,3-dimethyl-2,3-dihydro-1H- pyrrolo[3,2-b]pyridine-1-carbonyl)-3,9- diazaspiro[5.5]undecan-3-yl)methyl)-3- fluorobenzonitrile | 2.15 | 462.2 | B |
| 166 | 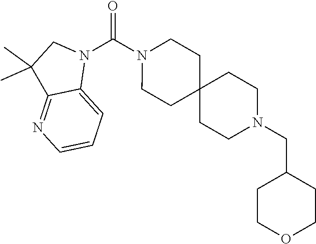 [SMILES: CC1(C)CN(C(=O)N2CCC3(CCN(CC4CCOCC4)CC3)CC2)c2cccnc21] | (3,3-dimethyl-2,3-dihydro-1H-pyrrolo[3,2- b]pyridin-1-yl)(9-((tetrahydro-2H-pyran-4- yl)methyl)-3,9-diazaspiro[5.5]undecan-3- yl)methanone | 1.48 | 427.1 | C |
| 167 | 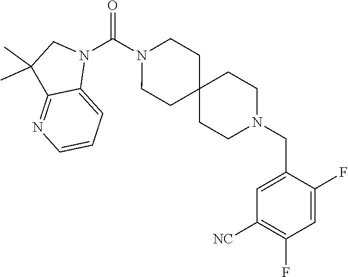 [SMILES: CC1(C)CN(C(=O)N2CCC3(CCN(Cc4cc(C#N)c(F)cc4F)CC3)CC2)c2cccnc21] | 5-((9-(3,3-dimethyl-2,3-dihydro-1H- pyrrolo[3,2-b]pyridine-1-carbonyl)-3,9- diazaspiro[5.5]undecan-3-yl)methyl)-2,4- difluorobenzonitrile | 2.16 | 480.0 | C |

### Example 168. ((1r,4r)-4-((3,5-Difluorobenzyl)(methyl)amino)cyclohexyl)(3,3,5-trimethyl-2,3-dihydro-1H-pyrrolo[3,2-b]pyridin-1-yl)methanone

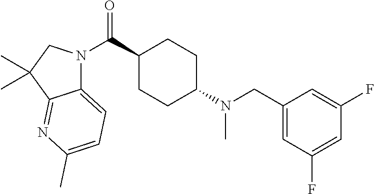 [SMILES: Cc1ccc2c(n1)C(C)(C)CN2C(=O)[C@H]1CC[C@H](N(C)Cc2cc(F)cc(F)c2)CC1]

Following the experimental procedure described in Example 123, starting from Intermediate 4D (50 mg, 0.31 mmol) and Intermediate 5 (105 mg, 0.37 mmol), the title compound was obtained (66 mg, 51% yield).

HPLC retention time (Method C): 2.58 min; MS: 428.3 (M+H).

This method was used for the preparation of Examples 169-176 using suitable starting

| | | | Ret | | |
|---|---|---|---|---|---|
| | | | time | MS | |
| EX | Structure | Chemical name | (min) | (M + H) | Method |
| 169 | 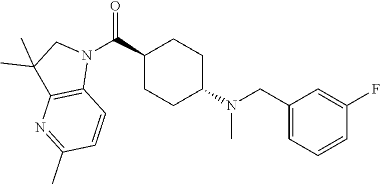 [SMILES: Cc1ccc2c(n1)C(C)(C)CN2C(=O)[C@H]1CC[C@H](N(C)Cc2cccc(F)c2)CC1] | ((1r,4r)-4-((3- fluorobenzyl)(methyl)amino)cyclohexyl)(3,3,5- trimethyl-2,3-dihydro-1H-pyrrolo[3,2- b]pyridin-1-yl)methanone | 2.41 | 410.2 | B |
| 170 | 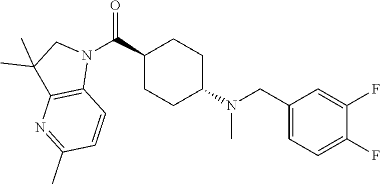 [SMILES: Cc1ccc2c(n1)C(C)(C)CN2C(=O)[C@H]1CC[C@H](N(C)Cc2ccc(F)c(F)c2)CC1] | ((1r,4r)-4-((3,4- difluorobenzyl)(methyl)amino)cyclohexyl)(3,3,5- trimethyl-2,3-dihydro-1H-pyrrolo[3,2- b]pyridin-1-yl)methanone | 2.48 | 428.3 | C |
| 171 | 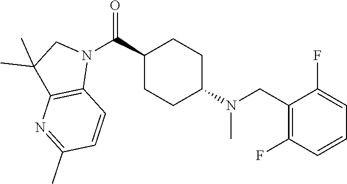 [SMILES: Cc1ccc2c(n1)C(C)(C)CN2C(=O)[C@H]1CC[C@H](N(C)Cc2c(F)cccc2F)CC1] | ((1r,4r)-4-((2,6- difluorobenzyl)(methyl)amino)cyclohexyl)(3,3,5- trimethyl-2,3-dihydro-1H-pyrrolo[3,2- b]pyridin-1-yl)methanone | 2.33 | 428.3 | C |
| 172 | 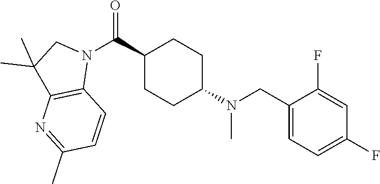 [SMILES: Cc1ccc2c(n1)C(C)(C)CN2C(=O)[C@H]1CC[C@H](N(C)Cc2ccc(F)cc2F)CC1] | ((1r,4r)-4-((2,4- difluorobenzyl)(methyl)amino)cyclohexyl)(3,3,5- trimethyl-2,3-dihydro-1H-pyrrolo[3,2- b]pyridin-1-yl)methanone | 2.41 | 428.3 | C |
| 173 | 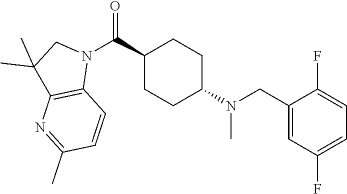 [SMILES: Cc1ccc2c(n1)C(C)(C)CN2C(=O)[C@H]1CC[C@H](N(C)Cc2cc(F)ccc2F)CC1] | ((1r,4r)-4-((2,5- difluorobenzyl)(methyl)amino)cyclohexyl)(3,3,5- trimethyl-2,3-dihydro-1H-pyrrolo[3,2- b]pyridin-1-yl)methanone | 2.48 | 428.3 | C |
| 174 | 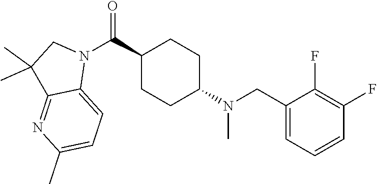 [SMILES: Cc1ccc2c(n1)C(C)(C)CN2C(=O)[C@H]1CC[C@H](N(C)Cc2cccc(F)c2F)CC1] | ((1r,4r)-4-((2,3- difluorobenzyl)(methyl)amino)cyclohexyl)(3,3 ,5-trimethyl-2,3-dihydro-1H-pyrrolo[3,2- b]pyridin-1-yl)methanone | 2.45 | 428.3 | C |
| 175 | 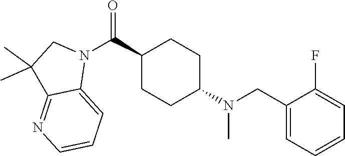 [SMILES: CN(Cc1ccccc1F)[C@H]1CC[C@H](C(=O)N2CC(C)(C)c3ncccc32)CC1] | (3,3-dimethyl-2,3-dihydro-1H-pyrrolo[3,2- b]pyridin-1-yl)((1r,4r)-4-((2- fluorobenzyl)(methyl)amino)cyclohexyl) methanone | 2.30 | 396.2 | B |
| 176 | 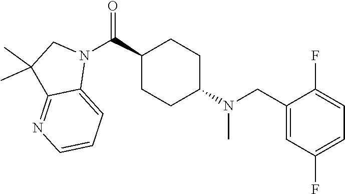 [SMILES: CN(Cc1cc(F)ccc1F)[C@H]1CC[C@H](C(=O)N2CC(C)(C)c3ncccc32)CC1] | ((1r,4r)-4-((2,5- difluorobenzyl)(methyl)amino)cyclohexyl)(3,3- dimethyl-2,3-dihydro-1H-pyrrolo[3,2- b]pyridin-1-yl)methanone | 2.36 | 414.0 | C |

### Example 177. ((1r,4r)-4-(Methyl((2-(trifluoromethyl)pyridin-4-yl)methyl)amino)cyclohexyl)(3,3,5-trimethyl-2,3-dihydro-1H-pyrrolo[3,2-b]pyridin-1-yl)methanone

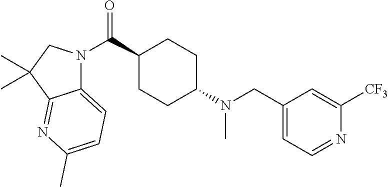 [SMILES: Cc1ccc2c(n1)C(C)(C)CN2C(=O)[C@H]1CC[C@H](N(C)Cc2ccnc(C(F)(F)F)c2)CC1]

Step 1. ((1r,4r)-4-(Methylamino)cyclohexyl)(3,3,5-trimethyl-2,3-dihydro-1H-pyrrolo[3,2-b]pyridin-1-yl)methanone: A solution of the compound obtained in Example 140 (0.66 g, 1.68 mmol) in MeOH (15 mL) was purged with N₂ in a pressure tube. Palladium (358 mg, 10% wt. on charcoal, wet) was added. The tube was purged with H₂ and the reaction mixture was stirred at r.t. overnight. The catalyst was filtered off and the solvent was evaporated to give the title compound (342 mg, 67% yield).

Step 2. Title compound: Following the experimental procedure described in step 3 of Example 79, starting from the product described in step 1 (36 mg, 0.12 mmol) and 2-(trifluoromethyl)isonicotinaldehyde (27 mg, 0.15 mmol), the title compound was obtained (12 mg, 22% yield).

HPLC retention time (Method C): 2.34 min; MS: 461.3 (M+H).

This method was used for the preparation of Examples 178 and 179 using suitable starting materials:

| | | | Ret | | |
|---|---|---|---|---|---|
| | | | time | MS | |
| EX | Structure | Chemical name | (min) | (M + H) | Method |
| 178 | 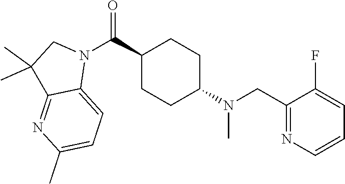 [SMILES: Cc1ccc2c(n1)C(C)(C)CN2C(=O)[C@H]1CC[C@H](N(C)Cc2ncccc2F)CC1] | ((1r,4r)-4-(((3-fluoropyridin-2- yl)methyl)(methyl)amino)cyclohexyl)(3,3,5- trimethyl-2,3-dihydro-1H-pyrrolo[3,2- b]pyridin-1-yl)methanone | 1.88 | 411.2 | B |
| 179 | 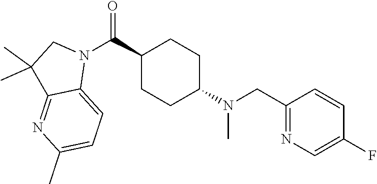 [SMILES: Cc1ccc2c(n1)C(C)(C)CN2C(=O)[C@H]1CC[C@H](N(C)Cc2ccc(F)cn2)CC1] | ((1r,4r)-4-(((5-fluoropyridin-2- yl)methyl)(methyl)amino)cyclohexyl)(3,3,5- trimethyl-2,3-dihydro-1H-pyrrolo[3,2- b]pyridin-1-yl)methanone | 1.99 | 411.2 | B |

### Example 180. 2-Fluoro-4-((methyl((1r,4r)-4-(3,3,5-trimethyl-2,3-dihydro-1H-pyrrolo[3,2-b]pyridine-1-carbonyl)cyclohexyl)amino)methyl)benzonitrile

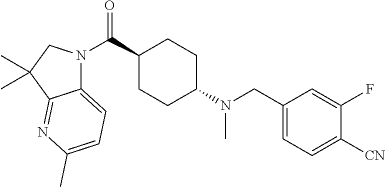 [SMILES: Cc1ccc2c(n1)C(C)(C)CN2C(=O)[C@H]1CC[C@H](N(C)Cc2ccc(C#N)c(F)c2)CC1]

Following the experimental procedure described in step 3 of Example 109, starting from the product described in step 1 of Example 177 (30 mg, 0.1 mmol) and 4-(bromomethyl)-2-fluorobenzonitrile (25 mg, 0.12 mmol), the title compound was obtained (21 mg, 49% yield).

HPLC retention time (Method B): 2.37 min; MS: 435.2 (M+H).

This method was used for the preparation of Examples 181-185 using suitable starting

| | | | Ret | | |
|---|---|---|---|---|---|
| | | | time | MS | |
| EX | Structure | Chemical name | (min) | (M + H) | Method |
| 181 | 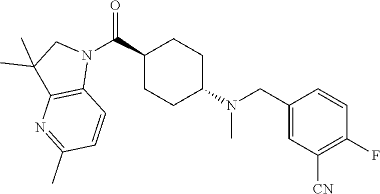 [SMILES: Cc1ccc2c(n1)C(C)(C)CN2C(=O)[C@H]1CC[C@H](N(C)Cc2ccc(F)c(C#N)c2)CC1] | 2-fluoro-5-((methyl((1r,4r)-4-(3,3,5-trimethyl- 2,3-dihydro-1H-pyrrolo[3,2-b]pyridine-1- carbonyl)cyclohexyl)amino)methyl)benzonitrile | 2.30 | 435.2 | B |
| 182 | 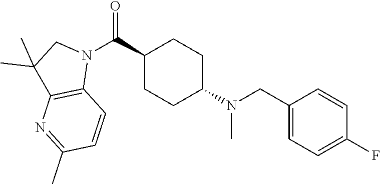 [SMILES: Cc1ccc2c(n1)C(C)(C)CN2C(=O)[C@H]1CC[C@H](N(C)Cc2ccc(F)cc2)CC1] | ((1r,4r)-4-((4- fluorobenzyl)(methyl)amino)cyclohexyl)(3,3,5- trimethyl-2,3-dihydro-1H-pyrrolo[3,2- b]pyridin-1-yl)methanone | 2.31 | 410.2 | B |
| 183 | 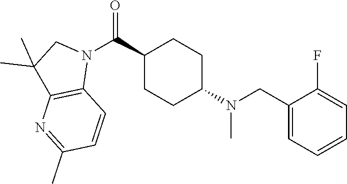 [SMILES: Cc1ccc2c(n1)C(C)(C)CN2C(=O)[C@H]1CC[C@H](N(C)Cc2ccccc2F)CC1] | ((1r,4r)-4-((2- fluorobenzyl)(methyl)amino)cyclohexyl)(3,3,5- trimethyl-2,3-dihydro-1H-pyrrolo[3,2- b]pyridin-1-yl)methanone | 2.32 | 410.2 | B |
| 184 | 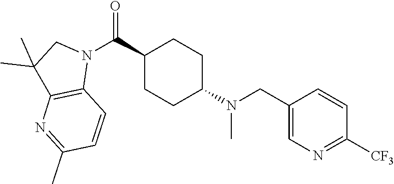 [SMILES: Cc1ccc2c(n1)C(C)(C)CN2C(=O)[C@H]1CC[C@H](N(C)Cc2ccc(C(F)(F)F)nc2)CC1] | ((1r,4r)-4-(methyl((6-(trifluoromethyl)pyridin- 3-yl)methyl)amino)cyclohexyl)(3,3,5- trimethyl-2,3-dihydro-1H-pyrrolo[3,2- b]pyridin-1-yl)methanone | 2.30 | 461.2 | B |
| 185 | 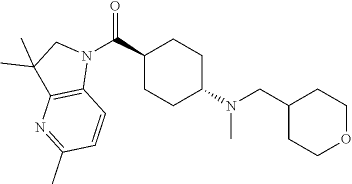 [SMILES: Cc1ccc2c(n1)C(C)(C)CN2C(=O)[C@H]1CC[C@H](N(C)CC2CCOCC2)CC1] | ((1r,4r)-4-(methyl((tetrahydro-2H-pyran-4- yl)methyl)amino)cyclohexyl)(3,3,5-trimethyl- 2,3-dihydro-1H-pyrrolo[3,2-b]pyridin-1- yl)methanone | 1.64 | 400.2 | B |

### Example 186. ((1r,4r)-4-(Benzyl(methyl)amino)cyclohexyl)(3,3-dimethyl-5-(trifluoromethyl)-2,3-dihydro-1H-pyrrolo[3,2-b]pyridin-1-yl)methanone

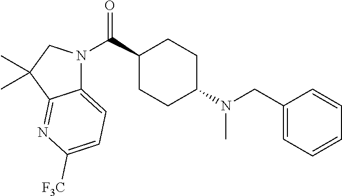 [SMILES: CN(Cc1ccccc1)[C@H]1CC[C@H](C(=O)N2CC(C)(C)c3nc(C(F)(F)F)ccc32)CC1]

To a solution of (1r,4r)-4-(benzyl(methyl)amino)cyclohexane-1-carboxylic acid (57 mg, 0.23 mmol) in DCM (3 mL) and DMF (2 drops), SOCl₂ (0.1 mL, 1.4 mmol) was added and the solution was stirred at 60° C. during 3 h. After this time, the solvent was removed under reduced pressure, the residue was redissolved in THF and a solution of Intermediate 4C (49 mg, 0.23 mmol) and TEA (0.06 mL, 0.46 mmol) was added. The resulting mixture was stirred at r.t. overnight. The solvent was evaporated and the residue was partitioned between EtOAc and aq. sat. NaHCO₃. The aqueous layer was back-extracted with EtOAc. The combined organic extracts were dried over MgSO₄, filtered and concentrated to dryness to afford the title compound (22 mg, 22% yield).

HPLC retention time (Method B): 2.69 min; MS: 446.4 (M+H).

This method was used for the preparation of Example 187 using suitable starting materials:

| | | | Ret | | |
|---|---|---|---|---|---|
| | | | time | MS | |
| EX | Structure | Chemical name | (min) | (M + H) | Method |
| 187 | 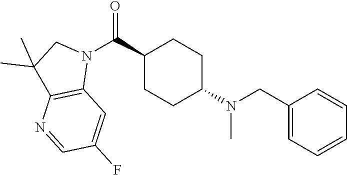 [SMILES: CN(Cc1ccccc1)[C@H]1CC[C@H](C(=O)N2CC(C)(C)c3ncc(F)cc32)CC1] | ((1r,4r)-4- (benzyl(methyl)amino)cyclohexyl)(6-fluoro- 3,3-dimethyl-2,3-dihydro-1H-pyrrolo[3,2- b]pyridin-1-yl)methanone | 2.39 | 396.2 | C |

### Example 188. ((1r,4r)-4-((2-Fluorobenzyl)amino)cyclohexyl)(3,3,5-trimethyl-2,3-dihydro-1H-pyrrolo[3,2-b]pyridin-1-yl)methanone

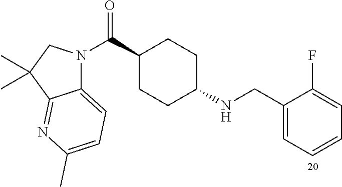 [SMILES: Cc1ccc2c(n1)C(C)(C)CN2C(=O)[C@H]1CC[C@H](NCc2ccccc2F)CC1]

Step 1. tert-Butyl ((1r,4r)-4-(3,3,5-trimethyl-2,3-dihydro-1H-pyrrolo[3,2-b]pyridine-1-carbonyl)cyclohexyl)carbamate: Following the experimental procedure described in Example 123, starting from (1r,4r)-4-((tert-butoxycarbonyl)amino)cyclohexane-1-carboxylic acid (360 mg, 1.48 mmol) and Intermediate 4D (200 mg, 1.23 mmol), the title compound was obtained (845 mg, 95% yield).

Step 2: ((1r,4r)-4-Aminocyclohexyl)(3,3,5-trimethyl-2,3-dihydro-1H-pyrrolo[3,2-b]pyridin-1-yl)methanone: Following the experimental procedure described in step 2 of Example 130, starting from the product described in step 1 (477 mg, 1.23 mmol) the title compound was obtained (382 mg, 80% yield).

Step 3. Title compound: To a solution of the product obtained in Step 2 (100 mg, 0.35 mmol) in DCE (3 mL), 2-fluorobenzaldehyde (43 mg, 0.35 mmol) and NaBH(OAc)₃ (147 mg, 0.7 mmol) were added under a N₂ atmosphere and the mixture was stirred under MW irradiation at 120° C. during 5 min. The residue was dissolved in DCM and the solution was washed with water. The aqueous layer was back-extracted with DCM. The combined organic extracts were dried over Na₂SO₄, filtered and concentrated to dryness. The residue was purified by flash chromatography, silica gel, gradient DCM to MeOH:DCM, to give the title compound (29.5 mg, 21% yield).

HPLC retention time (Method B): 2.04 min; MS: 396.2 (M+H).

### Pharmacological Study

This invention is aimed at providing a series of compounds which show pharmacological activity towards the σ₁ receptor and/or σ₂ receptor and, especially, compounds which have a binding expressed as Kᵢ responding to the following scales:
Kᵢ(σ₁) is preferably <1000 nM, more preferably <500 nM, even more preferably <100 nM; and
Kᵢ(σ₂) is preferably <1000 nM, more preferably <500 nM, even more preferably <100 nM.

### Human σ₁ Receptor Radioligand Assay

Transfected HEK-293 membranes (7 μg) were incubated with 5 nM of [³H](+)-pentazocine in assay buffer containing Tris-HCl 50 mM at pH 8. NBS (non-specific binding) was measured by adding 10 μM haloperidol. The binding of the test compound was measured at either one concentration (% inhibition at 1 or 10 □M) or five different concentrations to determine affinity values (Ki). Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH7.4). Filters were dried and counted at approximately 40% efficiency in a MicroBeta scintillation counter (Perkin-Elmer) using EcoScint liquid scintillation cocktail.

### Binding Assay to Human σ₂/TMEM97 Receptor.

Transfected HEK-293 membranes (15 μg) were incubated with 10 nM [³H]-1,3-Di-o-tolylguanidine (DTG) in assay buffer containing Tris-HCl 50 mM at pH 8.0. NSB (non-specific binding) was measured by adding 10 μM haloperidol. The binding of the test compound was measured at either one concentration (% inhibition at 1 or 10 □M) or five different concentrations to determine affinity values (Ki). Plates were incubated at 25° C. for 120 minutes. After the incubation period, the reaction mix was transferred to MultiScreen HTS, FC plates (Millipore), filtered and washed 3 times with ice-cold 10 mM Tris-HCL (pH 8.0). Filters were dried and counted at approximately 40% efficiency in a MicroBeta scintillation counter (Perkin-Elmer) using EcoScint liquid scintillation cocktail.

### Results:

The following scale has been adopted for representing the binding to σ₁-receptor expressed as Kᵢ:
+ Kᵢ (σ₁)>1000 nM or inhibition ranges between 1% and 50%.
++ 500 nM<=Kᵢ(σ₁)<=1000 nM+
+++ 100 nM<=Kᵢ(σ₁)<=500 nM+
++++Kᵢ(σ₁)<100 nM

The following scale has been adopted for representing the binding to σ2-receptor expressed as Kᵢ:
+ Kᵢ (σ₂)>1000 nM or inhibition ranges between 1% and 50%.
++500 nM<=Kᵢ(σ₂)<=1000 nM+
+++100 nM<=Kᵢ(σ₂)<=500 nM+
++++Kᵢ(σ₂)<100 nM

The results of the compounds showing binding for the σ- [symbol] and/or σ-□ receptor are shown in Table 1:

TABLE 1

| Example | Binding σ-□ | Binding σ-□ |
|---|---|---|
| 1 | ++++ | + |
| 2 | ++++ | + |
| 3 | +++ | + |
| 4 | ++++ | + |
| 5 | +++ | + |
| 6 | ++ | + |
| 7 | +++ | + |
| 8 | ++ | + |
| 9 | ++++ | + |
| 10 | +++ | + |
| 11 | +++ | ++++ |
| 12 | ++++ | + |
| 13 | ++++ | + |
| 14 | +++ | + |
| 15 | ++++ | + |
| 16 | ++++ | + |
| 17 | ++++ | + |
| 18 | +++ | +++ |
| 19 | +++ | + |
| 20 | ++++ | + |
| 21 | +++ | + |
| 22 | ++++ | ++ |
| 23 | +++ | + |
| 24 | ++++ | + |
| 25 | ++++ | +++ |
| 26 | ++++ | + |
| 27 | +++ | ++++ |
| 28 | +++ | + |
| 29 | +++ | ++++ |
| 30 | ++++ | |
| 31 | ++++ | ++ |
| 32 | ++++ | + |
| 33 | ++++ | + |
| 34 | +++ | + |
| 35 | +++ | + |
| 36 | +++ | ++ |
| 37 | +++ | ++++ |
| 38 | +++ | ++++ |
| 39 | +++ | + |
| 40 | ++++ | ++++ |
| 41 | +++ | + |
| 42 | +++ | + |
| 43 | ++ | + |
| 44 | ++++ | + |
| 45 | + | ++++ |
| 46 | ++++ | + |
| 47 | ++++ | + |
| 48 | ++++ | + |
| 49 | +++ | +++ |
| 50 | +++ | ++++ |
| 51 | ++++ | +++ |
| 52 | +++ | ++++ |
| 53 | +++ | +++ |
| 54 | +++ | ++++ |
| 55 | ++++ | +++ |
| 56 | + | ++ |
| 57 | ++++ | ++++ |
| 58 | +++ | +++ |
| 59 | ++ | ++++ |
| 60 | +++ | ++++ |
| 61 | + | ++++ |
| 62 | + | ++ |
| 63 | + | + |
| 64 | ++++ | ++++ |
| 65 | +++ | ++++ |
| 66 | +++ | +++ |
| 67 | ++++ | ++++ |
| 68 | +++ | ++++ |
| 69 | +++ | +++ |
| 70 | + | +++ |
| 71 | + | ++++ |
| 72 | ++++ | ++++ |
| 73 | + | ++ |
| 74 | + | ++ |
| 75 | + | ++ |
| 76 | + | ++ |
| 77 | + | +++ |
| 78 | ++ | ++++ |
| 79 | +++ | ++++ |
| 80 | +++ | ++++ |
| 81 | ++++ | ++ |
| 82 | +++ | ++++ |
| 83 | + | +++ |
| 84 | +++ | ++++ |
| 85 | ++ | +++ |
| 86 | ++ | ++++ |
| 87 | ++ | ++++ |
| 88 | +++ | + |
| 89 | ++++ | ++++ |
| 90 | +++ | ++++ |
| 91 | ++ | ++++ |
| 92 | + | ++++ |
| 93 | +++ | +++ |
| 94 | + | +++ |
| 95 | + | ++++ |
| 96 | +++ | +++ |
| 97 | ++ | + |
| 98 | + | ++++ |
| 99 | + | ++++ |
| 100 | ++ | ++++ |
| 101 | + | +++ |
| 102 | + | ++++ |
| 103 | +++ | ++++ |
| 104 | +++ | +++ |
| 105 | ++ | ++++ |
| 106 | ++ | +++ |
| 107 | + | ++++ |
| 108 | + | ++++ |
| 109 | +++ | ++++ |
| 110 | + | ++++ |
| 111 | +++ | + |
| 112 | +++ | + |
| 113 | +++ | + |
| 114 | +++ | ++++ |
| 115 | + | +++ |
| 116 | ++ | ++++ |
| 117 | +++ | ++++ |
| 118 | + | ++++ |
| 119 | +++ | +++ |
| 120 | ++ | ++++ |
| 121 | +++ | +++ |
| 122 | ++++ | +++ |
| 123 | ++++ | ++ |
| 124 | ++++ | + |
| 125 | ++++ | +++ |
| 126 | ++++ | ++++ |
| 127 | ++++ | ++++ |
| 128 | ++++ | +++ |
| 130 | ++++ | +++ |
| 131 | ++++ | + |
| 132 | ++++ | +++ |
| 133 | ++++ | + |
| 134 | +++ | + |
| 135 | ++++ | ++ |
| 136 | +++ | +++ |
| 137 | ++++ | +++ |
| 138 | ++++ | ++++ |
| 139 | +++ | +++ |
| 140 | ++++ | ++++ |
| 141 | ++++ | ++++ |
| 142 | ++++ | ++++ |
| 143 | +++ | ++++ |
| 144 | +++ | ++++ |
| 145 | ++++ | ++++ |
| 146 | ++++ | +++ |
| 147 | ++++ | ++++ |
| 148 | ++++ | ++++ |
| 149 | ++++ | ++++ |
| 150 | + | +++ |
| 151 | ++++ | ++++ |
| 152 | ++++ | ++++ |
| 153 | + | +++ |
| 154 | +++ | ++++ |
| 155 | + | ++++ |
| 156 | +++ | ++++ |
| 157 | +++ | ++++ |
| 158 | ++++ | ++++ |
| 159 | ++++ | +++ |
| 160 | ++++ | ++++ |
| 161 | +++ | ++++ |
| 162 | +++ | +++ |
| 163 | +++ | +++ |
| 164 | ++++ | ++++ |
| 165 | ++++ | ++++ |
| 166 | +++ | ++++ |
| 167 | ++++ | ++++ |
| 168 | ++++ | ++++ |
| 169 | ++++ | ++++ |
| 170 | ++++ | ++++ |
| 171 | ++++ | ++++ |
| 172 | ++++ | ++++ |
| 173 | ++++ | ++++ |
| 174 | ++++ | ++++ |
| 175 | ++++ | ++++ |
| 176 | ++++ | ++++ |
| 177 | +++ | ++++ |
| 178 | ++++ | +++ |
| 179 | ++++ | +++ |
| 180 | ++++ | ++++ |
| 181 | ++++ | ++++ |
| 182 | ++++ | ++++ |
| 183 | ++++ | ++++ |
| 184 | ++++ | ++++ |
| 185 | +++ | ++++ |
| 186 | ++++ | + |
| 187 | ++++ | ++++ |
| 188 | ++++ | ++++ |

1-16. (canceled)

17. A compound of formula (I): 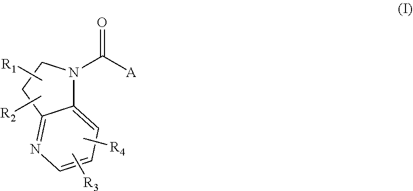
or a corresponding salt, a co-crystal, or a prodrug thereof, or a corresponding solvate thereof, wherein
R₁ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₂ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₃ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl, CN and OR₃′, wherein R₃′ is unsubstituted or substituted C₁₋₆ alkyl or hydrogen;
R₄ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl and CN;
A is a linear or cyclic amine selected from the group consisting of: 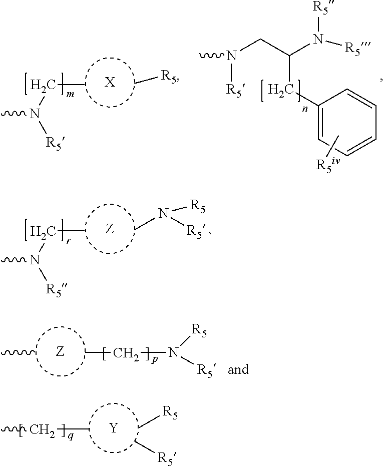
wherein
X is a N-containing heterocyclyl wherein said heterocyclyl is a saturated heterocyclyl;
Y is a N-containing heterocyclyl, wherein said heterocyclyl is a saturated heterocyclyl containing 1 to 2 nitrogen atoms;
Z is a C₄₋₆-cycloalkyl or a N-containing heterocyclyl wherein said heterocyclyl is a saturated heterocyclyl;
m is 0, 1 or 2;
n is 0, 1 or 2;
p is 0, 1 or 2;
q is 0, 1 or 2;
r is 0, 1 or 2;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl and substituted or unsubstituted alkylheterocyclyl;
R₅′ is selected from the group consisting of hydrogen and substituted or unsubstituted C₁₋₆ alkyl;
R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
alternatively, R₅″ and R₅′″ may form, together with the nitrogen atom to which they are attached, a substituted or unsubstituted N-containing heterocyclyl; and
R₅^{iv} is selected from the group consisting of hydrogen, halogen and OR₆, wherein R₆ is substituted or unsubstituted C₁₋₆ alkyl or a hydrogen.

18. The compound according to claim 17, wherein the compound is of the formula (Ia): 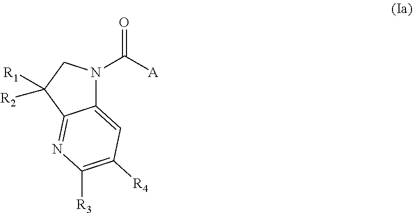
wherein R₁, R₂, R₃, R₄ and A are as defined in claim 1.

19. The compound according to claim 17, wherein
R₁ is selected from the group consisting of hydrogen and unsubstituted or substituted C₁₋₆ alkyl;
R₂ is selected from the group consisting of hydrogen and unsubstituted or substituted C₁₋₆ alkyl;
R₃ is selected from the group consisting of hydrogen, halogen, unsubstituted or substituted C₁₋₆ alkyl, CN and OR₃′, wherein R₃′ is unsubstituted or substituted C₁₋₆ alkyl; and
R₄ is selected from the group consisting of hydrogen, halogen, unsubstituted or substituted C1-6 alkyl and CN.

20. The compound according to claim 17, wherein the alkyl as defined in R₁-R₅^{iv}, if substituted, is substituted with one or more substituents selected from the group consisting of —OR′, halogen, —CN, haloalkyl, haloalkoxy, and —NR′R″, wherein each R′ and R″ is selected independently from hydrogen and unsubstituted C₁₋₆ alkyl.

21. The compound according to claim 17, wherein the alkyl, as defined in R₅, if substituted, is substituted with one or more substituents selected from unsubstituted C₁₋₆ alkyl and —OR′, wherein R′ is selected from the group consisting of hydrogen and unsubstituted C₁₋₆ alkyl.

22. The compound according to claim 17, wherein, the aryl in alkylaryl as defined in R₅, if substituted, is substituted with one or more substituents selected from the group consisting of halogen, —CN, —SO₂R′, —OR′, —NR′R″ and —CONR′R″, wherein each R′ and R″ is independently selected from the group consisting of hydrogen and unsubstituted C₁₋₆ alkyl.

23. The compound according to claim 22, wherein R₅ is benzyl.

24. The compound according to claim 1, wherein said compound is selected from the group consisting of:
N-(1-Benzylpiperidin-4-yl)-3,3,5-trimethyl-2,3-dihydro-1H-pyrrolo[3,2-b]pyridine-1-carboxamide;
N-(2-(dimethylamino)-2-phenylethyl)-3,3,5-trimethyl-2,3-dihydro-1H-pyrrolo[3,2-b]pyridine-1-carboxamide;
N-(2-(dimethylamino)-2-phenylethyl)-3,3-dimethyl-5-(trifluoromethyl)-2,3-dihydro-1H-pyrrolo[3,2-b]pyridine-1-carboxamide;
(R)—N-(2-(dimethylamino)-2-phenylethyl)-3,3,5-trimethyl-2,3-dihydro-1H-pyrrolo[3,2-b]pyridine-1-carboxamide;
(R)—N-(2-(dimethylamino)-2-phenylethyl)-3,3-dimethyl-2,3-dihydro-1H-pyrrolo[3,2-b]pyridine-1-carboxamide;
(R)—N-(2-(dimethylamino)-2-phenylethyl)-5-methyl-2,3-dihydro-1H-pyrrolo[3,2-b]pyridine-1-carboxamide;
3,3,5-trimethyl-N-(2-phenyl-2-(pyrrolidin-1-yl)ethyl)-2,3-dihydro-1H-pyrrolo[3,2-b]pyridine-1-carboxamide;
(R)—N-(2-(dimethylamino)-2-phenylethyl)-N,3,3,5-tetramethyl-2,3-dihydro-1H-pyrrolo[3,2-b]pyridine-1-carboxamide;
N-(2-(diethylamino)-2-phenylethyl)-3,3,5-trimethyl-2,3-dihydro-1H-pyrrolo[3,2-b]pyridine-1-carboxamide;
(4-benzylpiperazin-1-yl)(3,3,5-trimethyl-2,3-dihydro-1H-pyrrolo[3,2-b]pyridin-1-yl)methanone;
(4-(benzyl(methyl)amino)piperidin-1-yl)(3,3,5-trimethyl-2,3-dihydro-1H-pyrrolo[3,2-b]pyridin-1-yl)methanone
(S)—N-(2-(dimethylamino)-3-phenylpropyl)-3,3,5-trimethyl-2,3-dihydro-1H-pyrrolo[3,2-b]pyridine-1-carboxamide;
N-(2-(dimethylamino)-2-(4-methoxyphenyl)ethyl)-3,3,5-trimethyl-2,3-dihydro-1H-pyrrolo[3,2-b]pyridine-1-carboxamide;
(R)-6-chloro-N-(2-(dimethylamino)-2-phenylethyl)-3,3-dimethyl-2,3-dihydro-1H-pyrrolo[3,2-b]pyridine-1-carboxamide;
(R)-5-cyano-N-(2-(dimethylamino)-2-phenylethyl)-3,3-dimethyl-2,3-dihydro-1H-pyrrolo[3,2-b]pyridine-1-carboxamide;
(R)—N-(2-(dimethylamino)-2-phenylethyl)-6-fluoro-3,3,5-trimethyl-2,3-dihydro-1H-pyrrolo[3,2-b]pyridine-1-carboxamide;
(R)—N-(2-(dimethylamino)-2-phenylethyl)-5-methoxy-3,3-dimethyl-2,3-dihydro-1H-pyrrolo[3,2-b]pyridine-1-carboxamide;
(R)—N-(1-benzylpyrrolidin-3-yl)-3,3,5-trimethyl-2,3-dihydro-1H-pyrrolo[3,2-b]pyridine-1-carboxamide;
N-(2-(dimethylamino)-2-(2-fluorophenyl)ethyl)-3,3,5-trimethyl-2,3-dihydro-1H-pyrrolo[3,2-b]pyridine-1-carboxamide;
(R)—N-(2-(dimethylamino)-2-phenylethyl)-6-fluoro-3,3-dimethyl-2,3-dihydro-1H-pyrrolo[3,2-b]pyridine-1-carboxamide;
(S)—N-(1-benzylpyrrolidin-3-yl)-3,3,5-trimethyl-2,3-dihydro-1H-pyrrolo[3,2-b]pyridine-1-carboxamide;
N-(1-(4-fluorobenzyl)piperidin-4-yl)-3,3,5-trimethyl-2,3-dihydro-1H-pyrrolo[3,2-b]pyridine-1-carboxamide;
N-(1-(3-cyanobenzyl)piperidin-4-yl)-3,3,5-trimethyl-2,3-dihydro-1H-pyrrolo[3,2-b]pyridine-1-carboxamide;
N-(1-(4-cyanobenzyl)piperidin-4-yl)-3,3,5-trimethyl-2,3-dihydro-1H-pyrrolo[3,2-b]pyridine-1-carboxamide;
N-(1-isopentylpiperidin-4-yl)-3,3,5-trimethyl-2,3-dihydro-1H-pyrrolo[3,2-b]pyridine-1-carboxamide;
N-(1-(3-fluorobenzyl)piperidin-4-yl)-3,3,5-trimethyl-2,3-dihydro-1H-pyrrolo[3,2-b]pyridine-1-carboxamide;
3,3,5-trimethyl-N-(1-phenethylpiperidin-4-yl)-2,3-dihydro-1H-pyrrolo[3,2-b]pyridine-1-carboxamide;
N-(1-(2-ethoxyethyl)piperidin-4-yl)-3,3,5-trimethyl-2,3-dihydro-1H-pyrrolo[3,2-b]pyridine-1-carboxamide;
(4-(benzyl(methyl)amino)piperidin-1-yl)(3,3-dimethyl-2,3-dihydro-1H-pyrrolo[3,2-b]pyridin-1-yl)methanone;
N-(1-benzylpiperidin-4-yl)-5-cyano-3,3-dimethyl-2,3-dihydro-1H-pyrrolo[3,2-b]pyridine-1-carboxamide;
N-(1-benzylpiperidin-4-yl)-6-fluoro-3,3-dimethyl-2,3-dihydro-1H-pyrrolo[3,2-b]pyridine-1-carboxamide;
N-(1-benzylpiperidin-4-yl)-3,3-dimethyl-2,3-dihydro-1H-pyrrolo[3,2-b]pyridine-1-carboxamide;
(R)—N-(2-(dimethylamino)-2-(4-fluorophenyl)ethyl)-3,3,5-trimethyl-2,3-dihydro-1H-pyrrolo[3,2-b]pyridine-1-carboxamide;
(R)—N-(2-(dimethylamino)-2-(3-fluorophenyl)ethyl)-3,3,5-trimethyl-2,3-dihydro-1H-pyrrolo[3,2-b]pyridine-1-carboxamide;
(R)—N-(2-(dimethylamino)-2-(3-methoxyphenyl)ethyl)-3,3,5-trimethyl-2,3-dihydro-1H-pyrrolo[3,2-b]pyridine-1-carboxamide;
((3 aR,6aS)-5-benzylhexahydropyrrolo[3,4-c]pyrrol-2(1H)-yl)(3,3,5-trimethyl-2,3-dihydro-1H-pyrrolo[3,2-b]pyridin-1-yl)methanone;
(7-benzyl-2,7-diazaspiro[4.4]nonan-2-yl)(3,3,5-trimethyl-2,3-dihydro-1H-pyrrolo[3,2-b]pyridin-1-yl)methanone;
(2-benzyl-2,8-diazaspiro[4.5]decan-8-yl)(3,3,5-trimethyl-2,3-dihydro-1H-pyrrolo[3,2-b]pyridin-1-yl)methanone;
(S)-(3-(benzyl(methyl)amino)pyrrolidin-1-yl)(3,3,5-trimethyl-2,3-dihydro-1H-pyrrolo[3,2-b]pyridin-1-yl)methanone;
1-(4-(benzyl(methyl)amino)piperidine-1-carbonyl)-3,3-dimethyl-2,3-dihydro-1H-pyrrolo[3,2-b]pyridine-5-carbonitrile;
N-(1-isobutylpiperidin-4-yl)-3,3,5-trimethyl-2,3-dihydro-1H-pyrrolo[3,2-b]pyridine-1-carboxamide;
3,3,5-trimethyl-N-(1-((tetrahydro-2H-pyran-4-yl)methyl)piperidin-4-yl)-2,3-dihydro-1H-pyrrolo[3,2-b]pyridine-1-carboxamide;
(R)-(3-(benzyl(methyl)amino)pyrrolidin-1-yl)(3,3,5-trimethyl-2,3-dihydro-1H-pyrrolo[3,2-b]pyridin-1-yl)methanone;
N-(1-(3,4-difluorobenzyl)piperidin-4-yl)-3,3,5-trimethyl-2,3-dihydro-1H-pyrrolo[3,2-b]pyridine-1-carboxamide;
5-(((1-(3,3-dimethyl-2,3-dihydro-1H-pyrrolo[3,2-b]pyridine-1-carbonyl)piperidin-4-yl)(methyl)amino)methyl)-2-fluorobenzonitrile;
N-(1-(3,4-difluorobenzyl)piperidin-4-yl)-3,3-dimethyl-2,3-dihydro-1H-pyrrolo[3,2-b]pyridine-1-carboxamide;
N-(1-(3-fluorobenzyl)piperidin-4-yl)-3,3-dimethyl-2,3-dihydro-1H-pyrrolo[3,2-b]pyridine-1-carboxamide;
N-(1-(4-fluorobenzyl)piperidin-4-yl)-3,3-dimethyl-2,3-dihydro-1H-pyrrolo[3,2-b]pyridine-1-carboxamide;
(4-(benzyl(methyl)amino)piperidin-1-yl)(5-methyl-2,3-dihydro-1H-pyrrolo[3,2-b]pyridin-1-yl)methanone;
(4-(methyl(phenethyl)amino)piperidin-1-yl)(3,3,5-trimethyl-2,3-dihydro-1H-pyrrolo[3,2-b]pyridin-1-yl)methanone;
(4-(benzyl(methyl)amino)piperidin-1-yl)(2,3-dihydro-1H-pyrrolo[3,2-b]pyridin-1-yl)methanone;
(3,3-dimethyl-2,3-dihydro-1H-pyrrolo[3,2-b]pyridin-1-yl)(4-(methyl(phenethyl)amino)piperidin-1-yl)methanone;
(S)-(2,3-dihydro-1H-pyrrolo[3,2-b]pyridin-1-yl)(3-(methyl(phenethyl)amino)piperidin-1-yl)methanone;
(S)-(3-(methyl(phenethyl)amino)piperidin-1-yl)(3,3,5-trimethyl-2,3-dihydro-1H-pyrrolo[3,2-b]pyridin-1-yl)methanone;
N-(1-(3-chlorobenzyl)piperidin-4-yl)-3,3,5-trimethyl-2,3-dihydro-1H-pyrrolo[3,2-b]pyridine-1-carboxamide;
(4-(methyl(3-(methylsulfonyl)benzyl)amino)piperidin-1-yl)(3,3,5-trimethyl-2,3-dihydro-1H-pyrrolo[3,2-b]pyridin-1-yl)methanone;
(9-benzyl-3,9-diazaspiro [5.5]undecan-3-yl)(3,3,5-trimethyl-2,3-dihydro-1H-pyrrolo[3,2-b]pyridin-1-yl)methanone;
(4-((4-methoxybenzyl)(methyl)amino)piperidin-1-yl)(3,3,5-trimethyl-2,3-dihydro-1H-pyrrolo[3,2-b]pyridin-1-yl)methanone;
(4-((3-methoxybenzyl)(methyl)amino)piperidin-1-yl)(3,3,5-trimethyl-2,3-dihydro-1H-pyrrolo[3,2-b]pyridin-1-yl)methanone;
2-fluoro-5-((methyl(1-(3,3,5-trimethyl-2,3-dihydro-1H-pyrrolo[3,2-b]pyridine-1-carbonyl)piperidin-4-yl)amino)methyl)benzonitrile;
(S)-(3-(benzyl(methyl)amino)piperidin-1-yl)(2,3-dihydro-1H-pyrrolo[3,2-b]pyridin-1-yl)methanone;
(S)-(2,3-dihydro-1H-pyrrolo[3,2-b]pyridin-1-yl)(3-(methyl(phenethyl)amino)pyrrolidin-1-yl)methanone;
N,N-dimethyl-3-((methyl(1-(3,3,5-trimethyl-2,3-dihydro-1H-pyrrolo[3,2-b]pyridine-1-carbonyl)piperidin-4-yl)amino)methyl)benzamide;
(S)-(2,3-dihydro-1H-pyrrolo[3,2-b]pyridin-1-yl)(3-(isopentyl(methyl)amino)piperidin-1-yl)methanone;
(4-(methyl((tetrahydro-2H-pyran-4-yl)methyl)amino)piperidin-1-yl)(3,3,5-trimethyl-2,3-dihydro-1H-pyrrolo[3,2-b]pyridin-1-yl)methanone;
(4-((benzyl(methyl)amino)methyl)piperidin-1-yl)(3,3,5-trimethyl-2,3-dihydro-1H-pyrrolo[3,2-b]pyridin-1-yl)methanone;
(4-(isopentyl(methyl)amino)piperidin-1-yl)(3,3,5-trimethyl-2,3-dihydro-1H-pyrrolo[3,2-b]pyridin-1-yl)methanone;
(4-((4-(dimethylamino)benzyl)(methyl)amino)piperidin-1-yl)(3,3,5-trimethyl-2,3-dihydro-1H-pyrrolo[3,2-b]pyridin-1-yl)methanone;
N-((1-benzylpiperidin-4-yl)methyl)-3,3-dimethyl-2,3-dihydro-1H-pyrrolo[3,2-b]pyridine-1-carboxamide;
3,3-dimethyl-N-((1-phenethylpiperidin-4-yl)methyl)-2,3-dihydro-1H-pyrrolo[3,2-b]pyridine-1-carboxamide;
N-((1r,4r)-4-(benzyl(methyl)amino)cyclohexyl)-3,3-dimethyl-2,3-dihydro-1H-pyrrolo[3,2-b]pyridine-1-carboxamide;
N-((1s,4s)-4-(benzyl(methyl)amino)cyclohexyl)-3,3-dimethyl-2,3-dihydro-1H-pyrrolo[3,2-b]pyridine-1-carboxamide;
(4-(methyl(pyridin-2-ylmethyl)amino)piperidin-1-yl)(3,3,5-trimethyl-2,3-dihydro-1H-pyrrolo[3,2-b]pyridin-1-yl)methanone;
(4-(methyl(pyridin-3-ylmethyl)amino)piperidin-1-yl)(3,3,5-trimethyl-2,3-dihydro-1H-pyrrolo[3,2-b]pyridin-1-yl)methanone;
(4-(methyl(pyridin-4-ylmethyl)amino)piperidin-1-yl)(3,3,5-trimethyl-2,3-dihydro-1H-pyrrolo[3,2-b]pyridin-1-yl)methanone;
3-(((1-(3,3-dimethyl-2,3-dihydro-1H-pyrrolo[3,2-b]pyridine-1-carbonyl)piperidin-4-yl)(methyl)amino)methyl)-5-fluorobenzonitrile;
3-(((1-(3,3-dimethyl-2,3-dihydro-1H-pyrrolo[3,2-b]pyridine-1-carbonyl)piperidin-4-yl)(methyl)amino)methyl)-4-fluorobenzonitrile;
N-((1r,4r)-4-(benzyl(methyl)amino)cyclohexyl)-N,3,3-trimethyl-2,3-dihydro-1H-pyrrolo[3,2-b]pyridine-1-carboxamide;
(4-((3,4-Difluorobenzyl)(methyl)amino)piperidin-1-yl)(3,3-dimethyl-2,3-dihydro-1H-pyrrolo[3,2-b]pyridin-1-yl)methanone;
(8-benzyl-2,8-diazaspiro[4.5]decan-2-yl)(3,3,5-trimethyl-2,3-dihydro-1H-pyrrolo[3,2-b]pyridin-1-yl)methanone;
(6-benzyl-2,6-diazaspiro[3.3]heptan-2-yl)(3,3,5-trimethyl-2,3-dihydro-1H-pyrrolo[3,2-b]pyridin-1-yl)methanone;
N-((1-benzylazetidin-3-yl)methyl)-3,3,5-trimethyl-2,3-dihydro-1H-pyrrolo[3,2-b]pyridine-1-carboxamide;
3-(((1-(3,3-dimethyl-2,3-dihydro-1H-pyrrolo[3,2-b]pyridine-1-carbonyl)piperidin-4-yl)(methyl)amino)methyl)benzonitrile;
4-(((1-(3,3-dimethyl-2,3-dihydro-1H-pyrrolo[3,2-b]pyridine-1-carbonyl)piperidin-4-yl)(methyl)amino)methyl)benzonitrile;
(3,3-dimethyl-2,3-dihydro-1H-pyrrolo[3,2-b]pyridin-1-yl)(4-((3-fluorobenzyl)(methyl)amino)piperidin-1-yl)methanone;
(3,3-dimethyl-2,3-dihydro-1H-pyrrolo[3,2-b]pyridin-1-yl)(4-(4-fluorobenzyl)(methyl)amino)piperidin-1-yl)methanone;
4-(((1-(3,3-dimethyl-2,3-dihydro-1H-pyrrolo[3,2-b]pyridine-1-carbonyl)piperidin-4-yl)(methyl)amino)methyl)-2-fluorobenzonitrile;
N-(1-benzylazetidin-3-yl)-3,3-dimethyl-2,3-dihydro-1H-pyrrolo[3,2-b]pyridine-1-carboxamide;
(8-benzyl-2,8-diazaspiro[4.5]decan-2-yl)(3,3-dimethyl-2,3-dihydro-1H-pyrrolo[3,2-b]pyridin-1-yl)methanone;
3-((2-(3,3-dimethyl-2,3-dihydro-1H-pyrrolo[3,2-b]pyridine-1-carbonyl)-2,8-diazaspiro[4.5]decan-8-yl)methyl)benzonitrile;
(8-phenethyl-2,8-diazaspiro[4.5]decan-2-yl)(3,3,5-trimethyl-2,3-dihydro-1H-pyrrolo[3,2-b]pyridin-1-yl)methanone;
3-((2-(3,3,5-trimethyl-2,3-dihydro-1H-pyrrolo[3,2-b]pyridine-1-carbonyl)-2,8-diazaspiro[4.5]decan-8-yl)methyl)benzonitrile;
(2-benzyl-2,7-diazaspiro[3.5]nonan-7-yl)(3,3,5-trimethyl-2,3-dihydro-1H-pyrrolo[3,2-b]pyridin-1-yl)methanone;
(8-(pyridin-2-ylmethyl)-2,8-diazaspiro[4.5]decan-2-yl)(3,3,5-trimethyl-2,3-dihydro-1H-pyrrolo[3,2-b]pyridin-1-yl)methanone;
(8-(3-methoxybenzyl)-2,8-diazaspiro[4.5]decan-2-yl)(3,3,5-trimethyl-2,3-dihydro-1H-pyrrolo[3,2-b]pyridin-1-yl)methanone;
(8-(1-phenylethyl)-2,8-diazaspiro[4.5]decan-2-yl)(3,3,5-trimethyl-2,3-dihydro-1H-pyrrolo[3,2-b]pyridin-1-yl)methanone;
(S)-(2,3-dihydro-1H-pyrrolo[3,2-b]pyridin-1-yl)(3-(phenethylamino)pyrrolidin-1-yl)methanone;
(8-(pyridin-3-ylmethyl)-2,8-diazaspiro[4.5]decan-2-yl)(3,3,5-trimethyl-2,3-dihydro-1H-pyrrolo[3,2-b]pyridin-1-yl)methanone;
(8-(pyridin-4-ylmethyl)-2,8-diazaspiro[4.5]decan-2-yl)(3,3,5-trimethyl-2,3-dihydro-1H-pyrrolo[3,2-b]pyridin-1-yl)methanone;
(8-isopentyl-2,8-diazaspiro[4.5]decan-2-yl)(3,3,5-trimethyl-2,3-dihydro-1H-pyrrolo[3,2-b]pyridin-1-yl)methanone;
(S)-(2,3-dihydro-1H-pyrrolo[3,2-b]pyridin-1-yl)(3-(phenethylamino)piperidin-1-yl)methanone;
(8-(3-(methylsulfonyl)benzyl)-2,8-diazaspiro[4.5]decan-2-yl)(3,3,5-trimethyl-2,3-dihydro-1H-pyrrolo[3,2-b]pyridin-1-yl)methanone;
(8-(4-methoxybenzyl)-2,8-diazaspiro[4.5]decan-2-yl)(3,3,5-trimethyl-2,3-dihydro-1H-pyrrolo[3,2-b]pyridin-1-yl)methanone;
N-(7-benzyl-7-azaspiro[3.5]nonan-2-yl)-3,3-dimethyl-2,3-dihydro-1H-pyrrolo[3,2-b]pyridine-1-carboxamide;
(8-((tetrahydro-2H-pyran-4-yl)methyl)-2,8-diazaspiro[4.5]decan-2-yl)(3,3,5-trimethyl-2,3-dihydro-1H-pyrrolo[3,2-b]pyridin-1-yl)methanone;
N-((1-isopentylpiperidin-4-yl)methyl)-3,3-dimethyl-2,3-dihydro-1H-pyrrolo[3,2-b]pyridine-1-carboxamide;
2-fluoro-5-((2-(3,3,5-trimethyl-2,3-dihydro-1H-pyrrolo[3,2-b]pyridine-1-carbonyl)-2,8-diazaspiro[4.5]decan-8-yl)methyl)benzonitrile;
(8-(2-(tetrahydro-2H-pyran-4-yl)ethyl)-2,8-diazaspiro[4.5]decan-2-yl)(3,3,5-trimethyl-2,3-dihydro-1H-pyrrolo[3,2-b]pyridin-1-yl)methanone;
N-((1-(3,3-Dimethylbutyl)piperidin-4-yl)methyl)-3,3-dimethyl-2,3-dihydro-1H-pyrrolo[3,2-b]pyridine-1-carboxamide;
(8-(tetrahydro-2H-pyran-4-yl)-2,8-diazaspiro[4.5]decan-2-yl)(3,3,5-trimethyl-2,3-dihydro-1H-pyrrolo[3,2-b]pyridin-1-yl)methanone;
(S)-3,3,5-Trimethyl-N-(2-(methylamino)-2-phenylethyl)-2,3-dihydro-1H-pyrrolo[3,2-b]pyridine-1-carboxamide;
(R)-3,3,5-trimethyl-N-(2-(methylamino)-2-phenylethyl)-2,3-dihydro-1H-pyrrolo[3,2-b]pyridine-1-carboxamide;
(R)—N-(2-(ethylamino)-2-phenylethyl)-3,3,5-trimethyl-2,3-dihydro-1H-pyrrolo[3,2-b]pyridine-1-carboxamide;
(4-((4-Fluorobenzyl)(methyl)amino)piperidin-1-yl)(3,3,5-trimethyl-2,3-dihydro-1H-pyrrolo[3,2-b]pyridin-1-yl)methanone;
(4-(benzylamino)piperidin-1-yl)(3,3,5-trimethyl-2,3-dihydro-1H-pyrrolo[3,2-b]pyridin-1-yl)methanone;
(4-((3-fluorobenzyl)(methyl)amino)piperidin-1-yl)(3,3,5-trimethyl-2,3-dihydro-1H-pyrrolo[3,2-b]pyridin-1-yl)methanone;
4-((methyl(1-(3,3,5-trimethyl-2,3-dihydro-1H-pyrrolo[3,2-b]pyridine-1-carbonyl)piperidin-4-yl)amino)methyl)benzonitrile;
3-((methyl(1-(3,3,5-trimethyl-2,3-dihydro-1H-pyrrolo[3,2-b]pyridine-1-carbonyl)piperidin-4-yl)amino)methyl)benzonitrile;
(4-(isobutyl(methyl)amino)piperidin-1-yl)(3,3,5-trimethyl-2,3-dihydro-1H-pyrrolo[3,2-b]pyridin-1-yl)methanone
(3-(Isopentylamino)azepan-1-yl)(3,3,5-trimethyl-2,3-dihydro-1H-pyrrolo[3,2-b]pyridin-1-yl)methanone;
(S)-(2,3-dihydro-1H-pyrrolo[3,2-b]pyridin-1-yl)(3-(isopentylamino)azepan-1-yl)methanone;
(S)-(2,3-Dihydro-1H-pyrrolo[3,2-b]pyridin-1-yl)(3-(isopentyl(methyl)amino)azepan-1-yl)methanone;
(1-Benzylpiperidin-4-yl)(3,3,5-trimethyl-2,3-dihydro-1H-pyrrolo[3,2-b]pyridin-1-yl)methanone;
((3S,4S)-1-benzyl-4-methylpyrrolidin-3-yl)(3,3,5-trimethyl-2,3-dihydro-1H-pyrrolo[3,2-b]pyridin-1-yl)methanone;
((3R,4R)-1-benzyl-4-methylpyrrolidin-3-yl)(3,3,5-trimethyl-2,3-dihydro-1H-pyrrolo[3,2-b]pyridin-1-yl)methanone;
((1s,4s)-4-(benzyl(methyl)amino)cyclohexyl)(3,3,5-trimethyl-2,3-dihydro-1H-pyrrolo[3,2-b]pyridin-1-yl)methanone;
((1s,4s)-4-(benzyl(methyl)amino)cyclohexyl)(3,3-dimethyl-2,3-dihydro-1H-pyrrolo[3,2-b]pyridin-1-yl)methanone;
((1r,4r)-4-(Benzylamino)cyclohexyl)(3,3-dimethyl-2,3-dihydro-1H-pyrrolo[3,2-b]pyridin-1-yl)methanone dihydrochloride;
((1r,4r)-4-(benzylamino)cyclohexyl)(3,3,5-trimethyl-2,3-dihydro-1H-pyrrolo[3,2-b]pyridin-1-yl)methanone dihydrochloride;
2-(1-Benzylpiperidin-4-yl)-1-(3,3,5-trimethyl-2,3-dihydro-1H-pyrrolo[3,2-b]pyridin-1-yl)ethanone;
(1-benzylazetidin-3-yl)(3,3,5-trimethyl-2,3-dihydro-1H-pyrrolo[3,2-b]pyridin-1-yl)methanone;
(1-benzylazetidin-3-yl)(3,3-dimethyl-2,3-dihydro-1H-pyrrolo[3,2-b]pyridin-1-yl)methanone;
(1-(4-fluorobenzyl)azetidin-3-yl)(3,3,5-trimethyl-2,3-dihydro-1H-pyrrolo[3,2-b]pyridin-1-yl)methanone;
((1r,3r)-3-(benzylamino)cyclobutyl)(3,3,5-trimethyl-2,3-dihydro-1H-pyrrolo[3,2-b]pyridin-1-yl)methanone;
(1-(3-fluorobenzyl)azetidin-3-yl)(3,3,5-trimethyl-2,3-dihydro-1H-pyrrolo[3,2-b]pyridin-1-yl)methanone;
((1s,3s)-3-(benzylamino)cyclobutyl)(3,3,5-trimethyl-2,3-dihydro-1H-pyrrolo[3,2-b]pyridin-1-yl)methanone;
((1r,3r)-3-(Benzyl(methyl)amino)cyclobutyl)(3,3,5-trimethyl-2,3-dihydro-1H-pyrrolo[3,2-b]pyridin-1-yl)methanone;
((1r,4r)-4-(benzyl(methyl)amino)cyclohexyl)(3,3-dimethyl-2,3-dihydro-1H-pyrrolo[3,2-b]pyridin-1-yl)methanone;
((1s,3s)-3-(benzyl(methyl)amino)cyclobutyl)(3,3,5-trimethyl-2,3-dihydro-1H-pyrrolo[3,2-b]pyridin-1-yl)methanone;
((1r,4r)-4-(benzyl(methyl)amino)cyclohexyl)(3,3,5-trimethyl-2,3-dihydro-1H-pyrrolo[3,2-b]pyridin-1-yl)methanone;
(3,3-dimethyl-2,3-dihydro-1H-pyrrolo[3,2-b]pyridin-1-yl)(8-(2-fluorobenzyl)-2,8-diazaspiro[4.5]decan-2-yl)methanone;
4-((2-(3,3-dimethyl-2,3-dihydro-1H-pyrrolo[3,2-b]pyridine-1-carbonyl)-2,8-diazaspiro[4.5]decan-8-yl)methyl)-2-fluorobenzonitrile;
5-((2-(3,3-dimethyl-2,3-dihydro-1H-pyrrolo[3,2-b]pyridine-1-carbonyl)-2,8-diazaspiro[4.5]decan-8-yl)methyl)-2-fluorobenzonitrile;
(3,3-dimethyl-2,3-dihydro-1H-pyrrolo[3,2-b]pyridin-1-yl)(8-((tetrahydro-2H-pyran-4-yl)methyl)-2,8-diazaspiro[4.5]decan-2-yl)methanone;
(3,3-dimethyl-2,3-dihydro-1H-pyrrolo[3,2-b]pyridin-1-yl)(9-(2-fluorobenzyl)-3,9-diazaspiro [5 0.5]undecan-3-yl)methanone;
4-((9-(3,3-dimethyl-2,3-dihydro-1H-pyrrolo[3,2-b]pyridine-1-carbonyl)-3,9-diazaspiro [5 0.5]undecan-3-yl)methyl)-2-fluorobenzonitrile;
5-((9-(3,3-dimethyl-2,3-dihydro-1H-pyrrolo[3,2-b]pyridine-1-carbonyl)-3,9-diazaspiro [5 0.5]undecan-3-yl)methyl)-2-fluorobenzonitrile;
(8-(2,5-difluorobenzyl)-2,8-diazaspiro[4.5]decan-2-yl)(3,3-dimethyl-2,3-dihydro-1H-pyrrolo[3,2-b]pyridin-1-yl)methanone;
(3,3-dimethyl-2,3-dihydro-1H-pyrrolo[3,2-b]pyridin-1-yl)(8-(4-fluorobenzyl)-2,8-diazaspiro[4.5]decan-2-yl)methanone;
(8-(2,6-difluorobenzyl)-2,8-diazaspiro[4.5]decan-2-yl)(3,3-dimethyl-2,3-dihydro-1H-pyrrolo[3,2-b]pyridin-1-yl)methanone;
4-((2-(3,3-dimethyl-2,3-dihydro-1H-pyrrolo[3,2-b]pyridine-1-carbonyl)-2,8-diazaspiro[4.5]decan-8-yl)methyl)benzonitrile;
(3,3-dimethyl-2,3-dihydro-1H-pyrrolo[3,2-b]pyridin-1-yl)(8-(3-fluorobenzyl)-2,8-diazaspiro[4.5]decan-2-yl)methanone;
3,3-dimethyl-2,3-dihydro-1H-pyrrolo[3,2-b]pyridin-1-yl)(8-(3-fluoropyridin-2-yl)methyl)-2,8-diazaspiro[4.5]decan-2-yl)methanone;
(3,3-dimethyl-2,3-dihydro-1H-pyrrolo[3,2-b]pyridin-1-yl)(8-(5-fluoropyridin-2-yl)methyl)-2,8-diazaspiro[4.5]decan-2-yl)methanone;
(3,3-dimethyl-2,3-dihydro-1H-pyrrolo[3,2-b]pyridin-1-yl)(8-((6-(trifluoromethyl)pyridin-3-yl)methyl)-2,8-diazaspiro[4.5]decan-2-yl)methanone;
(3,3-dimethyl-2,3-dihydro-1H-pyrrolo[3,2-b]pyridin-1-yl)(8-(2-(tetrahydro-2H-pyran-4-yl)ethyl)-2,8-diazaspiro[4.5]decan-2-yl)methanone;
4-((2-(3,3-dimethyl-2,3-dihydro-1H-pyrrolo[3,2-b]pyridine-1-carbonyl)-2,8-diazaspiro[4.5]decan-8-yl)methyl)-3-fluorobenzonitrile;
5-((2-(3,3-dimethyl-2,3-dihydro-1H-pyrrolo[3,2-b]pyridine-1-carbonyl)-2,8-diazaspiro[4.5]decan-8-yl)methyl)-2,4-difluorobenzonitrile;
(7-benzyl-2,7-diazaspiro[3.5]nonan-2-yl)(3,3-dimethyl-2,3-dihydro-1H-pyrrolo[3,2-b]pyridin-1-yl)methanone;
(9-(2-fluorobenzyl)-3,9-diazaspiro[5.5]undecan-3-yl)(3,3,5-trimethyl-2,3-dihydro-1H-pyrrolo[3,2-b]pyridin-1-yl)methanone;
(9-((tetrahydro-2H-pyran-4-yl)methyl)-3,9-diazaspiro[5.5]undecan-3-yl)(3,3,5-trimethyl-2,3-dihydro-1H-pyrrolo[3,2-b]pyridin-1-yl)methanone;
2-fluoro-5-((9-(3,3,5-trimethyl-2,3-dihydro-1H-pyrrolo[3,2-b]pyridine-1-carbonyl)-3,9-diazaspiro[5.5]undecan-3-yl)methyl)benzonitrile;
2-fluoro-44(9-(3,3,5-trimethyl-2,3-dihydro-1H-pyrrolo[3,2-b]pyridine-1-carbonyl)-3,9-diazaspiro[5.5]undecan-3-yl)methyl)benzonitrile;
(9-(2,5-difluorobenzyl)-3,9-diazaspiro[5.5]undecan-3-yl)(3,3-dimethyl-2,3-dihydro-1H-pyrrolo[3,2-b]pyridin-1-yl)methanone;
4-((9-(3,3-dimethyl-2,3-dihydro-1H-pyrrolo[3,2-b]pyridine-1-carbonyl)-3,9-diazaspiro[5.5]undecan-3-yl)methyl)-3-fluorobenzonitrile;
(3,3-dimethyl-2,3-dihydro-1H-pyrrolo[3,2-b]pyridin-1-yl)(9-((tetrahydro-2H-pyran-4-yl)methyl)-3,9-diazaspiro[5.5]undecan-3-yl)methanone;
5-((9-(3,3-dimethyl-2,3-dihydro-1H-pyrrolo[3,2-b]pyridine-1-carbonyl)-3,9-diazaspiro[5.5]undecan-3-yl)methyl)-2,4-difluorobenzonitrile;
((1r,4r)-4-(3,5-difluorobenzyl)(methyl)amino)cyclohexyl)(3,3,5-trimethyl-2,3-dihydro-1H-pyrrolo[3,2-b]pyridin-1-yl)methanone;
((1r,4r)-4-(3-fluorobenzyl)(methyl)amino)cyclohexyl)(3,3,5-trimethyl-2,3-dihydro-1H-pyrrolo[3,2-b]pyridin-1-yl)methanone;
((1r,4r)-4-(3,4-difluorobenzyl)(methyl)amino)cyclohexyl)(3,3,5-trimethyl-2,3-dihydro-1H-pyrrolo[3,2-b]pyridin-1-yl)methanone;
((1r,4r)-4-(2,6-difluorobenzyl)(methyl)amino)cyclohexyl)(3,3,5-trimethyl-2,3-dihydro-1H-pyrrolo[3,2-b]pyridin-1-yl)methanone;
((1r,4r)-4-(2,4-difluorobenzyl)(methyl)amino)cyclohexyl)(3,3,5-trimethyl-2,3-dihydro-1H-pyrrolo[3,2-b]pyridin-1-yl)methanone;
((1r,4r)-4-((2,5-difluorobenzyl)(methyl)amino)cyclohexyl)(3,3,5-trimethyl-2,3-dihydro-1H-pyrrolo[3,2-b]pyridin-1-yl)methanone;
((1r,4r)-4-((2,3-difluorobenzyl)(methyl)amino)cyclohexyl)(3,3,5-trimethyl-2,3-dihydro-1H-pyrrolo[3,2-b]pyridin-1-yl)methanone;
(3,3-dimethyl-2,3-dihydro-1H-pyrrolo[3,2-b]pyridin-1-yl)((1 r,4r)-4-((2-fluorobenzyl)(methyl)amino)cyclohexyl)methanone;
((1r,4r)-4-((2,5-difluorobenzyl)(methyl)amino)cyclohexyl)(3,3-dimethyl-2,3-dihydro-1H-pyrrolo[3,2-b]pyridin-1-yl)methanone;
((1r,4r)-4-(methyl((2-(trifluoromethyl)pyridin-4-yl)methyl)amino)cyclohexyl)(3,3,5-trimethyl-2,3-dihydro-1H-pyrrolo[3,2-b]pyridin-1-yl)methanone;
((1r,4r)-4-(((3-fluoropyridin-2-yl)methyl)(methyl)amino)cyclohexyl)(3,3,5-trimethyl-2,3-dihydro-1H-pyrrolo[3,2-b]pyridin-1-yl)methanone;
((1r,4r)-4-(((5-fluoropyridin-2-yl)methyl)(methyl)amino)cyclohexyl)(3,3,5-trimethyl-2,3-dihydro-1H-pyrrolo[3,2-b]pyridin-1-yl)methanone;
2-fluoro-4-((methyl((1 r,4r)-4-(3,3,5-trimethyl-2,3-dihydro-1H-pyrrolo[3,2-b]pyridine-1-carbonyl)cyclohexyl)amino)methyl)benzonitrile;
2-fluoro-5-((methyl((1 r,4r)-4-(3,3,5-trimethyl-2,3-dihydro-1H-pyrrolo[3,2-b]pyridine-1-carbonyl)cyclohexyl)amino)methyl)benzonitrile;
((1r,4r)-4-((4-fluorobenzyl)(methyl)amino)cyclohexyl)(3,3,5-trimethyl-2,3-dihydro-1H-pyrrolo[3,2-b]pyridin-1-yl)methanone;
((1r,4r)-4-((2-fluorobenzyl)(methyl)amino)cyclohexyl)(3,3,5-trimethyl-2,3-dihydro-1H-pyrrolo[3,2-b]pyridin-1-yl)methanone;
((1r,4r)-4-(methyl((6-(trifluoromethyl)pyridin-3-yl)methyl)amino)cyclohexyl)(3,3,5-trimethyl-2,3-dihydro-1H-pyrrolo[3,2-b]pyridin-1-yl)methanone;
((1r,4r)-4-(methyl((tetrahydro-2H-pyran-4-yl)methyl)amino)cyclohexyl)(3,3,5-trimethyl-2,3-dihydro-1H-pyrrolo[3,2-b]pyridin-1-yl)methanone;
((1r,4r)-4-(benzyl(methyl)amino)cyclohexyl)(3,3-dimethyl-5-(trifluoromethyl)-2,3-dihydro-1H-pyrrolo[3,2-b]pyridin-1-yl)methanone;
((1r,4r)-4-(benzyl(methyl)amino)cyclohexyl)(6-fluoro-3,3-dimethyl-2,3-dihydro-1H-pyrrolo[3,2-b]pyridin-1-yl)methanone; and
((1r,4r)-4-((2-fluorobenzyl)amino)cyclohexyl)(3,3,5-trimethyl-2,3-dihydro-1H-pyrrolo[3,2-b]pyridin-1-yl)methanone.

25. A process for producing a compound of formula (I): 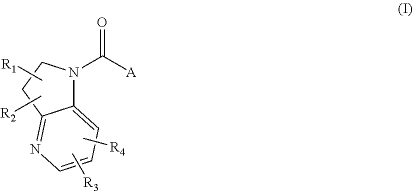
said process comprising treating a compound of formula (II) 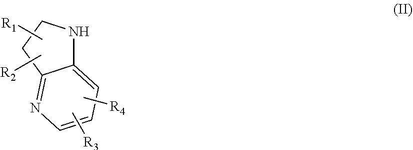
with an amine compound A in the presence of a carbonyl source under conditions sufficient to produce the compound of formula (I),
wherein
R₁ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₂ is selected from the group consisting of hydrogen, halogen, unsubstituted or substituted C₁₋₆ alkyl, substituted or unsubstituted C₂₋₆ alkenyl and substituted or unsubstituted C₂₋₆ alkynyl;
R₃ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl, CN and OR₃′, wherein R₃′ is unsubstituted or substituted C₁₋₆ alkyl or hydrogen;
R₄ is selected from the group consisting of hydrogen, halogen, unsubstituted or substituted C₁₋₆ alkyl, substituted or unsubstituted C₂₋₆ alkenyl, substituted or unsubstituted C₂₋₆ alkynyl, unsubstituted or substituted cycloalkyl, unsubstituted or substituted heterocyclyl and CN;
A is a linear or cyclic amine compound selected from the group consisting of: 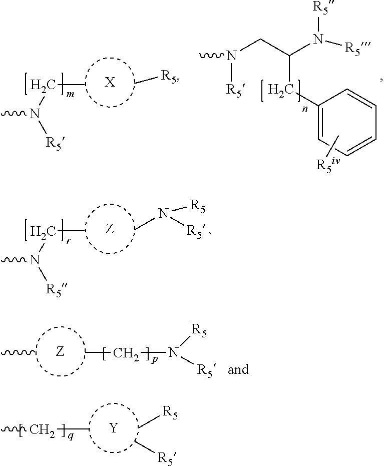
wherein
X is a N-containing heterocyclyl wherein said heterocyclyl is a saturated heterocyclyl;
Y is a N-containing heterocyclyl, wherein said heterocyclyl is a saturated heterocyclyl containing 1 to 2 nitrogen atoms;
Z is a N-containing heterocyclyl wherein said heterocyclyl is a saturated heterocyclyl;
m is 0, 1 or 2;
n is 0, 1 or 2;
p is 0, 1 or 2;
q is 0, 1 or 2;
r is 0, 1 or 2;
R₅ is selected from the group consisting of substituted or unsubstituted C₁₋₆ alkyl, substituted or unsubstituted C₂₋₆ alkenyl, substituted or unsubstituted C₂₋₆ alkynyl, substituted or unsubstituted alkylaryl, substituted or unsubstituted cycloalkyl, substituted or unsubstituted alkylcycloalkyl, substituted or unsubstituted heterocyclyl and substituted or unsubstituted alkylheterocyclyl;
R₅′ is selected from the group consisting of hydrogen and substituted or unsubstituted C₁₋₆ alkyl;
R₅″ and R₅′″ are independently selected from the group consisting of hydrogen, substituted or unsubstituted C₁₋₆ alkyl, substituted or unsubstituted C₂₋₆ alkenyl, and substituted or unsubstituted C₂₋₆ alkynyl;
alternatively, R₅″ and R₅′″ may form, together with the nitrogen atom to which they are attached, a substituted or unsubstituted N-containing heterocyclyl; and
R₅^{iv} is selected from the group consisting of hydrogen, halogen and OR₆, wherein R₆ is substituted or unsubstituted C₁₋₆ alkyl or a hydrogen.

26. The process of claim 25, wherein said carbonyl source comprises triphosgene, phosgene, 1,1′-carbonyldiimidazole, 1,1′-carbonylbisbenzotriazole, or a mixture thereof.

27. A process for producing a compound of claim 17, said process comprising:
treating a compound of formula (II) 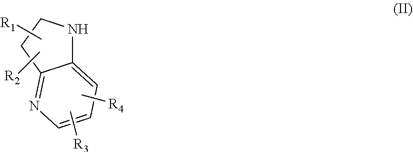
with a carboxylic acid compound of formula (IV) 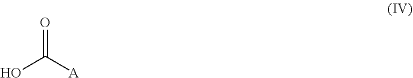
under conditions sufficient to produce the compound of formula (I),
wherein
R₁, R₂, R₃, R₄ and A are as defined in claim 17.

28. A method for treating a clinical condition mediated by a sigma receptor, said method comprising administering a therapeutically effective amount of a compound of claim 17 to a subject in need of such a treatment.

29. The method of claim 28, wherein said clinical condition is mediated by a sigma-1 receptor, sigma-2 receptor, or a combination thereof.

30. The method of claim 28, wherein said clinical condition comprises pain, depression, anxiety, attention-deficit-/hyperactivity disorder (ADHD), or a combination thereof.

31. The method of claim 30, wherein said pain comprises neuropathic pain, inflammatory pain, chronic pain, allodynia, hyperalgesia, or a combination thereof.

32. A pharmaceutical composition comprising:
a compound of claim 17 or a pharmaceutically acceptable salt, an isomer, a co-crystal, a prodrug or a solvate thereof; and
at least one of a pharmaceutically acceptable carrier, additive, adjuvant, or vehicle.

33. The pharmaceutical composition of claim 32, wherein said compound of claim 17 is present as an isomerically enriched mixture.

34. The pharmaceutical composition of claim 32, wherein said compound of claim 17 is present as a diastereomeric mixture.

35. The pharmaceutical composition of claim 32, wherein said compound of claim 17 is present as a racemic mixture.

36. The pharmaceutical composition of claim 32, wherein said compound of claim 17 is present as an enantiomerically enriched mixture.
